(19) United States
(12) Patent Application Publication
(10) Pub. No.: US 2025/0155449 A1
(43) Pub. Date: May 15, 2025

(54) RECOMBINANT ANTIBODIES COMPRISING ANTI-HUMAN IMMUNOGLOBULIN G-INVERTASE FUSION PROTEINS AND METHODS OF USING THE SAME

(71) Applicant: THE JOHNS HOPKINS UNIVERSITY, Baltimore, MD (US)

(72) Inventors: Elissa Kathleen LEONARD, Baltimore, MD (US); Miguel ALLER PELLITERO, Baltimore, MD (US); Boris D. JUELG, Cambridge, MA (US); Netzahualcoyotl ARROYO, Baltimore, MD (US); Jamie SPANGLER, Baltimore, MD (US)

(21) Appl. No.: 18/840,190

(22) PCT Filed: Feb. 21, 2023
(86) PCT No.: PCT/US2023/062931

§ 371 Date: Aug. 21, 2024

Related U.S. Application Data
(60) Provisional application No. 63/312,418, filed on Feb. 22, 2022.

(51) Int. Cl. G01N 33/68; C07K 16/46; C12N 9/26; C12Q 1/40; C12Q 1/54

(52) U.S. Cl. CPC G01N 33/6854; C07K 16/46; C12N 9/2431; C12Q 1/40; C12Q 1/54; C07K 2319/00; C12Y 302/01026; G01N 2333/165

(57) ABSTRACT

The present disclosure relates to recombinant antibodies comprising anti-human immunoglobulin G (IgG)-invertase fusion proteins (also called “recombinant antibody fusion proteins”), kits comprising the same, and methods of using the same. The recombinant antibody fusion proteins permit detection of antibodies to a broad array of antigenic polypeptides, such as a polypeptide of an infectious pathogen or a human polypeptide known to associate with autoimmune diseases or disorders in a quick, reliable, and affordable immunoassay using personal glucometers.

### CROSS REFERENCE TO RELATED APPLICATIONS

This application is a U.S. national stage application of PCT/US2023/062931, filed on 21 Feb. 2023, which claims the benefit of, and relies on the filing date of, U.S. provisional patent application No. 63/312,418, filed on Feb. 22, 2022, the entire disclosures of which are hereby incorporated by reference in their entireties.

### SEQUENCE LISTING

The instant application contains a Sequence Listing which has been filed electronically in XML format and is hereby incorporated by reference in its entirety. Said XML copy, created on 18 Feb. 2023, is named 0184_0196_PCT_SL.xml and is 50 kilobytes in size.

### FIELD

The present disclosure relates to recombinant antibodies comprising anti-human immunoglobulin G (IgG)-invertase fusion proteins (also called “recombinant antibody fusion proteins”), kits comprising the same, and methods of using the same. The recombinant antibody fusion proteins permit detection of antibodies to a broad array of antigenic polypeptides, such as a polypeptide of an infectious pathogen or a human polypeptide known to associate with autoimmune diseases or disorders in a quick, reliable, and affordable immunoassay using personal glucometers.

### BACKGROUND

The coronavirus disease of 2019 (COVID-19) pandemic has highlighted the importance of point-of-care, rapid diagnostic assessments that can immediately inform patients of disease risk in order to guide behavior and prevent disease spread (1, 2). While vaccination and prior infection provide some protection against symptomatic reinfection with the causative virus, severe acute respiratory syndrome coronavirus 2 (SARS-COV-2), the emergence of new variants and waning of immunity has rendered many susceptible to symptomatic disease. It is still unclear what variables contribute to the longevity, or the lack thereof, of immune protection, and the current data suggests that the factors may be highly individualized (3-5). Nonetheless, a person's level of SARS-COV-2-specific antibodies is correlated with protection against symptomatic disease (3, 4, 6, 7), and can therefore serve as an indicator of immune protection. Tests that measure levels of SARS-COV-2-specific antibodies are not widely available to the general public, as the existing assays require highly skilled technicians and specialized equipment, and the vast majority of these assays must be performed in specially-certified labs (8-10). These obstacles severely limit the use of pathogen-specific antibody detection tests, and thus impede characterization of immunity at the population scale, which is essential for making informed policy decisions concerning public safety measures and booster vaccinations.

Development of a rapid point-of-care diagnostic for serum antibody quantification that is broadly deployable, standardized, and that does not require specialized technical skills would provide the reliable, population-wide data required to deepen epidemiological understanding of pandemics such as COVID-19 (11). This knowledge would establish the level and durability of vaccine protection, which will be vital in constructing public health and vaccination recommendations in the near- and long-term. Furthermore, a test that can be used broadly and with consistent, accurate results would provide information about key differences in infection-versus vaccine-induced immunity, and how these differences may impact the spread of disease. Finally, at an individual level, a point-of-care antibody serology diagnostic would allow patients to assess their personal degree of disease susceptibility, empowering them to make informed decisions concerning lifestyle and preventative care.

Benchmark commercial antibody detection assays achieve high sensitivity by entrapping immunoglobulins (Igs) from human samples between the target antigen and a detection antibody, which is typically conjugated to a reporter enzyme. The concentration of immunoglobulins in clinical specimens is then determined spectrophotometrically by quantifying the product of the enzymatic reaction, which is directly proportional to the number of “sandwiched” immunoglobulins. Although several enzyme reporters have been described in the literature (12), current benchmark antibody assays use either horseradish peroxidase (HRP) or alkaline phosphatase (AP). These enzymes are easily isolated from plants (HRP) (13) or animal tissues (AP) (14), and achieve high catalytic rates (2,600 and 850 s⁻¹, respectively) (12), leading to intense optical signal outputs. Due to these favorable properties, HRP- and AP-based enzyme-linked immunosorbent assays (ELISAs) remain the standard techniques for serological testing (15). Commercial ELISA instrumentation is available in an array of formats and scales, ranging from portable instruments (e.g., manufactured by Samsung, Alere, and Eurolyser Diagnostica) to high throughput, multiplexed clinical analyzers (e.g., manufactured by Luminex) (15). However, the requirement for expensive high-quality optical equipment to achieve accurate antibody measurements often restricts the availability of such instruments to specialized laboratories. Translation of ELISA technology to the point of care has been demonstrated via lateral flow assays, but the results are qualitative at best (16). Thus, there remains a need to develop new diagnostic strategies that allow for detection and quantitative measurement of antibody titers at the point of care.

A rational strategy to achieve rapid, broadly-deployable, and easily-executable monitoring of antibody titers lies in coupling antibody assays with digital bioelectronic detectors, such as personal glucometers, which are commercially available at the population scale. Yi Lu and colleagues pioneered the concept of using glucometers as modular diagnostic devices (17, 18), highlighting the extensive clinical validation that over-the-counter glucometers have undergone in diabetes treatment, as well as their increasingly robust integration with mobile health solutions. By conjugating a detection aptamer to a reporter enzyme, Lu et al coupled detection of several target molecules to the production of glucose, rather than traditional colorimetric or fluorogenic products utilized by ELISAs. The concentration of glucose, as measured by the glucometer, was thus directly proportional to the number of analyte molecules present in the clinical sample. Several enzymes have been proposed for this detection approach (17); however, the biocatalyst invertase uniquely combines high catalytic rates (about 1540 s⁻¹, comparable to HRP and AP), thermal stability (up to 80° C.) and pH stability (pH=3-6), and high specificity for the substrate sucrose (19, 20).

Invertase-mediated conversion of sucrose has been integrated in recent molecular diagnostics (21-27), but coupling of invertase and the detection molecule (e.g., antibody) has proved difficult. Some studies have avoided direct attachment by conjugating both the detection antibody and invertase to the same nanoparticle (21, 22) or nanowire (23), whereas others have used streptavidin as an intermediate, and formed complexes of biotinylated antibody and biotinylated invertase (24). To avoid the steric issues that likely interfere with efficient chemical conjugation methods, invertase has been conjugated to a small nucleic acid aptamer (25, 26). Just one group has reported the direct, site-specific, enzyme-mediated conjugation of invertase to an antibody single-chain fragment (scFv) (27), and though this method had the advantage of ensuring a consistent ratio of invertase to detection molecule, conjugation efficiency was found to be very low. Thus, there remains a need for efficient and consistent chemical coupling of invertase for the invertase-mediated conversion of sucrose to be better integrated in molecular diagnostics.

### SUMMARY

Here, we designed a genetic fusion protein comprised of an antibody against the Fc domain of human IgG (anti-hIgG) and the enzyme invertase. Our fusion protein overcomes the issues of inefficient and inconsistent chemical coupling of invertase to detection molecules, which has previously hindered the development of glucometer-based detection systems. Our engineered antibody-enzyme fusion protein (denoted Ab+Inv) was produced with high yields in a mammalian expression system, and the resulting construct fully preserved human IgG binding and catalytic activity relative to unmodified proteins. We validated that a strip-based assay (FIG. 1) which employed this fusion protein was effective in distinguishing between human samples that did or did not contain SARS-COV-2-specific antibodies, with comparable sensitivity to gold standard commercial ELISA-based detection methods. Given the rapid, robust, and broadly deployable nature of the system we developed, this platform promises to promote the understanding and containment of the SARS-COV-2 virus. Furthermore, the versatile format allows for ready adaptation as a precision diagnostic to detect a wide range of disease and medical conditions.

Disclosed herein are recombinant antibodies comprising anti-human immunoglobulin G (IgG)-invertase fusion proteins (or “recombinant antibody fusion proteins”), kits comprising the same, and methods of using the same. Anti-human IgG-invertase fusion proteins of the disclosure comprise an anti-human IgG antibody fragment fused to an invertase, and a linker positioned between the anti-human IgG antibody fragment and the invertase. Accordingly, in one aspect, disclosed herein are polypeptides comprising an anti-human IgG antibody fragment fused to an invertase, and a linker positioned between the anti-human IgG antibody fragment and the invertase, wherein the anti-human IgG antibody fragment comprises an amino acid sequence having at least 90% sequence identity to the amino acid sequence of SEQ ID NO: 2 or SEQ ID NO: 4 and the invertase comprises an amino acid sequence having at least 90% sequence identity to the amino acid sequence of SEQ ID NO: 6. In some embodiments, the linker comprises an amino acid sequence having at least 90% sequence identity to the amino acid sequence of SEQ ID NO: 8, SEQ ID NO: 10, SEQ ID NO: 12, or SEQ ID NO: 14. In some embodiments, the polypeptide further comprises a signal peptide positioned at the N-terminus of the polypeptide. In some embodiments, the signal peptide comprises an amino acid sequence having at least 90% sequence identity to the amino acid sequence of SEQ ID NO: 16, SEQ ID NO: 18, or SEQ ID NO: 20. In some embodiments, the polypeptide comprises an amino acid sequence having at least 90% sequence identity to the amino acid sequence of SEQ ID NO: 26, SEQ ID NO: 28, SEQ ID NO: 30, or SEQ ID NO: 32. In some embodiments, the polypeptide comprises the amino acid sequence of SEQ ID NO: 26, SEQ ID NO: 28, SEQ ID NO: 30, or SEQ ID NO: 32.

Also provided are nucleic acid molecules that encode any of the anti-human IgG-invertase fusion polypeptides disclosed herein and vectors comprising such nucleic acid molecules. Host cells comprising any of the disclosed nucleic acid molecules or any of the disclosed vectors are also provided.

In some embodiments, the disclosure further relates to a recombinant antibody fusion protein comprising at least one anti-human IgG-invertase polypeptide disclosed herein. In some embodiments, the recombinant antibody fusion protein comprising 2 copies of the at least one anti-human IgG-invertase polypeptide disclosed herein. In some embodiments, the recombinant antibody fusion protein comprises 2 copies of the polypeptide comprising the amino acid sequence of SEQ ID NO: 26 and 2 copies of the anti-human IgG antibody fragment comprising the amino acid sequence of SEQ ID NO: 24. In some embodiments, the recombinant antibody fusion protein comprises 2 copies of the polypeptide comprising the amino acid sequence of SEQ ID NO: 28 and 2 copies of the anti-human IgG antibody fragment comprising the amino acid sequence of SEQ ID NO: 24. In some embodiments, the recombinant antibody fusion protein comprises 2 copies of the polypeptide comprising the amino acid sequence of SEQ ID NO: 30 and 2 copies of the anti-human IgG antibody fragment comprising the amino acid sequence of SEQ ID NO: 22. In some embodiments, the recombinant antibody fusion protein comprises 2 copies of the polypeptide comprising the amino acid sequence of SEQ ID NO: 32 and 2 copies of the anti-human IgG antibody fragment comprising the amino acid sequence of SEQ ID NO: 22. In some embodiments, the recombinant antibody fusion protein of the disclosure is capable of binding to a human IgG and has a catalytic activity of converting sucrose to glucose.

The disclosure also relates to a kit comprising any of the recombinant antibody fusion proteins disclosed herein and an antigenic polypeptide immobilized or absorbed onto a solid support. In some embodiments, the solid support is a strip, bead, plate, or slide. In some embodiments, the antigenic polypeptide is a pathogen-specific polypeptide. In some embodiments, the pathogen-specific polypeptide is a polypeptide of SARS-COV-2. In some embodiments, the kit of the disclosure further comprises a solution comprising sucrose. In some embodiments, the kit of the disclosure further comprises a glucometer. In some embodiments, the kit of the disclosure is a point-of-care diagnostic kit. In some embodiments, the kit of the disclosure is an over-the-counter diagnostic kit.

In another aspect, the disclosure relates to a method of detecting an antibody to an antigenic polypeptide in a sample using any of the disclosed recombinant antibody fusion proteins or a kit comprising the same. In any of these embodiments, the disclosed methods comprise: a) contacting the sample with an antigenic polypeptide, such as a polypeptide of a pathogen or a polypeptide of human original known to associate with autoimmune diseases or disorders, under conditions sufficient for the antibody to bind the antigenic polypeptide to produce a bound antibody; b) contacting the bound antibody with at least one recombinant antibody fusion protein of the disclosure to produce a binding complex, wherein the at least one recombinant antibody fusion protein is capable of binding to a human IgG and has an invertase catalytic activity of converting at least one substrate into at least one detectable product; c) contacting the binding complex with the at least one substrate under conditions sufficient to convert the at least one substrate into the at least one detectable product; and d) detecting the presence or absence of the at least one detectable product in the sample, wherein detecting the presence of the at least one detectable product in the sample indicates that the sample contains the antibody to the antigenic polypeptide or the subject has an infection of the pathogen. In some embodiments, the methods further comprise obtaining the sample from a subject. In some embodiments, the sample is an oral fluid, nasal fluid, saliva, blood, plasma, or serum.

In a further aspect, the disclosure relates to a method of diagnosing an infection of a pathogen in a subject in need thereof using any of the disclosed recombinant antibody fusion proteins or a kit comprising the same. In some embodiments, the disclosure relates to a method for treating a pathogenic infection in a subject in need thereof, the method comprising diagnosing the subject as having the pathogenic infection by any of the diagnosis method disclosed herein and administering to the subject a therapeutically effective amount of treatment for the pathogenic infection. In some embodiments, the treatment for the pathogenic infection comprises one or more of: a) resting; and b) administering to the subject a therapeutically effective amount of one or more antibiotics, antivirals, or antifungals.

### BRIEF DESCRIPTION OF THE DRAWINGS

The accompanying drawings, which are incorporated in and constitute a part of this specification, illustrate certain embodiments, and together with the written description, serve to explain certain principles of the methods and devices disclosed herein.

FIG. 1 depicts a schematic overview of detection assay to quantify COVID-19-specific antibodies using a commercially available glucometer, a non-limiting exemplary application of the antibody-invertase (Ab+Inv) fusion proteins disclosed herein. A strip coated with SARS-COV-2 spike protein RBD is incubated with patient serum, and any RBD-specific antibodies bind to the strip. The strip is rinsed to remove any non-specific antibodies and transferred to a solution containing Ab+Inv fusion protein, which binds to any patient antibody captured on the strip. The strip is again rinsed, and transferred to a solution of sucrose and incubated. The amount of sucrose converted to glucose is measured using a commercial glucometer and is proportional to the amount of patient antibody in the serum.

FIG. 2A-2D depict the design and purification of antibody-invertase (Ab+Inv) fusion proteins. FIG. 2A: Schematic of Ab+Inv fusion proteins containing an anti-human IgG antibody. The C-terminus of the antibody light chain (LC) was tethered to the N-terminus of invertase via a flexible linker 15 or 25 amino acids in length (LC15 and LC25, respectively), or the C terminus of the heavy chain (HC) is tethered to the N-terminus of invertase via a flexible linker 17 or 27 amino acids in length (HC17 and HC27, respectively). HC and LC variable and constant domains are labeled. FIG. 2B: Ab+Inv fusion proteins migrate at slightly larger molecular weights (MW) by SDS-PAGE. The unfused antibody (Ab) and invertase (Inv) alone run at the expected sizes under non-reducing (146 kDa for Ab and 62 kDa for Inv) and reducing (49 kDa for Ab HC, 24 kDa for Ab LC, and 62 kDa for Inv) conditions. Note that the samples for both SDS-PAGE gels were boiled to minimize the divergent migration of invertase and the fusion proteins relative to their expected molecular weight (62 kDa for invertase, and about 266 kDa for the Ab+Inv fusion proteins). The Ab+Inv fusion proteins run somewhat larger than their expected MW (about 266 kDa) under non-reducing conditions, as do the Inv-fused LC (˜84 kDa) and HC (˜110 kDa) under reducing conditions. FIG. 2C: The yield per liter of transfected cells for each Ab+Inv fusion protein is shown compared to the yield of the unfused antibody (Ab). For LC15, HC17, and HC27, the yield from the pooled peak 1 is shown in a lighter shade and the yield from the pooled peak 2 is shown in the darker shade. Bar height reflects the total yield for both peaks. FIG. 2D: Representative size-exclusion chromatography (SEC) traces show for the four Ab+Inv fusion proteins are shown. The separately pooled fractions that were tested for LC15, HC17, and HC27 (peak 1 and peak 2) are indicated.

FIG. 3A-3C depict that the Ab+Inv fusion proteins bind hIgG with the same affinity as unfused antibody. FIG. 3A: Equilibrium BLI titrations of soluble HP6017 antibody, Ab+Inv LC15, LC25, HC17, and HC27 against immobilized hIgG. Invertase (Inv) is included as a negative control. FIG. 3B: The Kp derived from the three-parameter curve fit of the equilibrium binding data in FIG. 3A are shown. FIG. 3C: The association rate (kon) generated from the kinetic BLI curve fit of the data in FIG. 9, assuming a 1:1 binding model, is shown.

FIG. 4A-4D depict that Ab+Inv fusion proteins exhibit equivalent catalytic activity to unfused invertase. FIG. 4A: Ab+Inv fusion proteins or invertase (Inv) were incubated with 250 mM sucrose for 6 to 24 minutes. Concentrations of 166 nM Ab+Inv fusion proteins and 330 nM for Inv were used to achieve a molar equivalent amount of enzyme, n=1. FIG. 4B: Various concentrations of unfused Ab, Ab+Inv fusion proteins, or Inv were incubated with 250 mM sucrose for 8 minutes. The concentration of the Ab+Inv fusion proteins is one half of the invertase concentration shown on the x-axis, as each fusion protein contains two copies of invertase, n=2. FIG. 4C: Unfused Ab, Ab+Inv fusion proteins, or Inv were incubated with various concentrations of sucrose for 15 minutes. Concentrations of 166 nM Ab+Inv fusion proteins and 330 nM for Inv were used to achieve a molar equivalent amount of enzyme, n=2. FIG. 4D: Unfused Ab, Ab+Inv fusion proteins, or Inv were incubated with 250 mM sucrose for 15 minutes. Concentrations of 166 nM Ab+Inv fusion proteins and 330 nM for Inv were used to achieve a molar equivalent amount of enzyme, n=3. The dotted lines in FIG. 4A-4D indicate the lower limit of detection for the glucometer (10 mg/dL). Error bars indicate standard deviation in all panels.

FIG. 5A-5F depict the glucometer-based immunoassay using antibody-invertase fusion protein accurately detects presence of SARS-COV-2-targeted antibodies in patient samples. FIG. 5A: Schematic of the detection workflow for glucometer-based strip immunoassay, which detects sucrose conversion by Ab+Inv fusion protein LC15 in proportion to SARS-COV-2 spike protein RBD-targeted antibodies in a test sample. FIG. 5B: The platform shown in FIG. 5A was used to build calibration curves, detecting glucose readouts for various concentrations of anti-SARS-COV-2 spike protein RBD antibody. Fitted single curve yields EC₅₀=35 nM, n=3. FIG. 5C: The platform shown in FIG. 5A was used to measure anti-SARS-COV-2 antibody titers in patient samples with known seroconversion status (TS1), n=3. Stratification between positive and negative samples was observed. FIG. 5D: Schematic of the detection workflow for a benchmark spectrophotometric strip immunoassay, which detects tetramethylbenzidine (TMB) oxidation by horse radish peroxidase (HRP)-labeled anti-hIgG antibody in proportion to SARS-COV-2 spike protein RBD-targeted antibodies in a test sample. FIG. 5E: The strategy shown in FIG. 5D was used to build calibration curves, detecting glucose readouts for various concentrations of anti-SARS-COV-2 spike protein RBD antibody. Fitted single curve yields EC₅₀=2 nM, n=3. FIG. 5F: The platform shown in FIG. 5D was used to measure anti-SARS-COV-2 antibody titers in patient samples with known seroconversion status (TS1), n=3. Discrimination between positive and negative samples matched that of the glucometer-based assay. Error bars indicate standard deviation in all panels.

FIG. 6 depicts the glucometer-based immunoassay using Ab+Inv fusion protein enables monitoring of anti-SARS-COV-2 antibody titers in hospitalized patients. A glucometer-based strip immunoassay was used to measure SARS-COV-2 spike protein RBD-targeted antibody levels in samples from hospitalized patients with COVID-19 (TS2), n=1. The glucometer assay consistently determined the emergence of anti-SARS-COV-2 IgG antibody responses at about 10 days post symptoms onset, and responses plateaued were observed at about 20 days post symptom onset in patients A, B, and E. Solid lines are included for visual clarity to highlight IgG titer trends.

FIG. 7A-7D depict the robust clinical agreement observed between glucometer-based and commercial spectrophotometric immunoassays. FIG. 7A: Longitudinal time course of SARS-COV-2 spike protein RBD-targeted antibody levels in hospitalized patient with COVID-19 (patient B from the TS2 cohort), as detected by glucometer-based immunoassay using Ab+Inv fusion protein, n=1. FIG. 7B: Longitudinal time course of SARS-COV-2 spike protein RBD-targeted antibody levels in hospitalized patient with COVID-19 (patient B from the TS2 cohort), as detected by a commercial spectrophotometric assay (Epitope Diagnostics™), n=1. Excellent qualitative agreement in shape, onset, and plateau of the antibody titer vs time curve was noted between the glucometer-based and commercial spectrophotometric immunoassays. FIG. 7C: To establish a seroconversion threshold for the glucometer-based assay, we computed the average and standard deviation of the glucose output generated by 5 confirmed SARS-COV-2 negative samples, obtaining a seroconversion cutoff of 39±32 ng/ml. Extrapolating this threshold to all the data from the TS2 cohort, we were able to accurately stratify by seroconversion status. FIG. 7D: Agreement chart generated using the Bangdiwala method, comparing glucometer-based measurements a commercial spectrophotometric assay (Coronacheck™). A 96% positive and 95% negative percent agreement was observed.

FIG. 8A-8F depict that the SDS-PAGE gel characterizations of Inv and Ab+Inv fusion proteins show some differences in SEC peak 1 and peak 2, as shown in FIG. 2C, but no significant difference in activity. FIG. 8A: Inv that has not been boiled (“NB”) migrates at a larger molecular weight than its expected molecular weight (MW) of 62 kDa. When boiled for 5 minutes (“B”), it migrates much more consistently, and appears as a much cleaner, more punctate band at the expected MW. FIG. 8B-8E: The fractions from the SEC purification of Ab+Inv LC15 (FIG. 8B), LC25 (FIG. 8C), HC17 (FIG. 8D), and HC27 (FIG. 8E) were not boiled. All show similar divergent migration to the unboiled Inv, running above the expected MW of about 266 kDa, and having a more diffuse MW distribution. Due to the distinct shoulder observed in the SEC trace for HC17 and HC27, and due to the higher presence of aggregate species apparent in the earlier fractions of LC15, fractions 8 to 11 (peak 1) of these three fusion proteins were pooled separately from fractions 12 to 18 (peak 2). Since there were no apparent differences between the fractions by SDS-PAGE for LC25, and it ran as a monodisperse peak, fractions 7 to 17 were pooled (peak ½). FIG. 8F: Peaks 1 and 2 of Ab+Inv LC15, HC17, HC27, and peak ½ of LC25 were incubated at 166 μM with 500 mM sucrose for 15 min, n=3. Error bars represent SD. The dotted line indicates the lower limit of detection for the glucometer (10 mg/dL).

FIG. 9A-9G depict the kinetic analysis of interaction of Ab+Inv fusion proteins with hIgG over time. Time-dependent binding traces obtained from bio-layer interferometry studies between immobilized hIgG and unfused Ab (FIG. 9A), Ab+Inv LC15 (FIG. 9B), LC25 (FIG. 9C), Inv (FIG. 9D), HC17 (FIG. 9E), or HC27 (FIG. 9F) are shown. Fitted parameters are presented in FIG. 9G.

FIG. 10 depicts the characterization of Ab+Inv fusion protein catalytic activity in the presence of soluble hIgG. Unfused Ab, Ab+Inv fusion proteins, or Inv were incubated with 250 mM sucrose for 15 minutes in the presence of 662 nM hIgG. Concentrations of 166 nM Ab+Inv fusion proteins and 330 nM of Inv were used to achieve a molar equivalent amount of enzyme, n=3. Error bars represent SD. The dotted line indicates the lower limit of detection for the glucometer (10 mg/dL).

FIG. 11A depicts a representative photograph of the laser-cut, poly(methyl methacrylate) immunoassay test strip used in Example 1. FIG. 11B depicts an vector image of such an immunoassay test strip.

FIG. 12A-12C depict the optimization of hIgG quantification using glucometer-based and spectrophotometric strip immunoassays. FIG. 12A: Loading of SARS-COV-2 spike protein RBD onto the plastic strips was calibrated, and saturation of the assay response was observed when RBD was deposited from solutions at >1 μM concentrations; thus, 1 μM RBD deposition solutions were used in all subsequent experiments, n=3. FIG. 12B: The concentration of the LC15 Ab+Inv fusion protein was optimized (reaction time with sucrose was 30 min), and a saturating concentration of 0.1 μM was used for all subsequent studies, n=3. FIG. 12C: The concentration of the HRP-modified secondary antibody was optimized (reaction time with TMB/hydrogen peroxide was 10 min), and a saturating concentration of 0.13 μM was used for subsequent studies, n=3. The commercial human monoclonal anti-SARS-COV-2 spike protein RBD primary antibody concentration was fixed at 13.6 nM.

FIG. 13 depicts the endpoint evaluation of catalytic activity for the Ab+Inv fusion proteins produced in Example 1. Sucrose-to-glucose conversion activity of Ab+Inv fusion proteins was measured by glucometer-based immunoassays using the indicated Ab+Inv fusion proteins. Human monoclonal anti-SARS-COV-2 spike protein RBD antibody concentration was fixed at 13.6 nM and invertase-labelled secondary antibody concentration was fixed at 0.1 μM. Sucrose concentration was set at 0.1 M and a reaction time of 30 minutes was used. All tests were performed in PBS (pH 7.4), n=3.

FIG. 14A-14B depict the hIgG titration in PBS using glucometer-based versus spectrophotometric immunoassays. FIG. 14A: Glucometer-based immunoassays were performed on various concentrations of human monoclonal anti-SARS-COV-2 spike protein RBD antibody using the Ab+Inv fusion protein LC15, n=3. FIG. 14B: Spectrophotometric immunoassays were performed on various concentrations of human monoclonal anti-SARS-COV-2 spike protein RBD antibody using HRP-labelled anti-hIgG antibody, n=3. Invertase-labelled secondary antibody concentration was fixed at 0.1 μM. Sucrose concentration was set at 0.1 M and a strip reaction time with sucrose solution of 45 minutes was used. HRP-labelled secondary antibody concentration was fixed at 0.13 μM. The TMB/hydrogen peroxide solution was acquired commercially, and the concentration is not disclosed by the vendor. A reaction time with TMB/hydrogen peroxide solution of 10 minutes was used. Curve fits yielded EC₅₀ values of 12 nM and 1 nM for glucometer-based and spectrophotometric assays, respectively.

FIG. 15 depicts the evaluation of the sensitivity of the strip immunoassay disclosed in Example 1 to human serum concentration. Spectrophotometric-based immunoassays were performed in the presence of varying dilutions of commercially purchased human serum. These measurements included the addition of 5% (w/w) casein to the serum. Strips were functionalized with 1 μM RBD and incubated in 5% (w/w) casein in PBS to prevent non-specific protein binding. The assays were performed using 13.6 nM of primary human monoclonal anti-SARS-COV-2 spike protein RBD antibody and 0.13 μM of HRP-modified anti hIgG secondary antibody. A strip reaction time with commercial TMB solution of 10 minutes was used, n=3.

FIG. 16 depicts the effect of casein and incubation time on the signaling output of the glucometer-based immunoassay. Glucometer-based immunoassays were performed on various concentrations of human monoclonal anti-SARS-COV-2 spike protein RBD antibody using the Ab+Inv fusion protein LC15 in commercial human serum (negative for SARS-COV-2). Filled symbols indicate assay performance in the absence of casein added to the serum samples using a 60 minutes strip incubation period in the sucrose solution, whereas open symbols indicate assay performance in the presence of 5% (w/w) casein in human serum using a longer strip incubation time in the sucrose solution of 60 minutes, n=3. Based on these results, all subsequent glucometer-based assays were conducted without casein and using an incubation time in sucrose solution of 60 minutes.

FIG. 17A-17D depicts strong agreement observed between glucometer-based assay and benchmark commercial immunoassays. Agreement charts (Bangdiwala method) compare the clinical agreement between the glucometer-based assay disclosed in Example 1 and various commercially available spectrophotometric assays that rely on the detection of anti-SARS-CoV-2 spike protein RBD IgGs: Coronacheck™ (FIG. 17A), BioMedomics (FIG. 17B), EDI™ (FIG. 17C), and QuantiVac™ (FIG. 17D).

FIG. 18 depicts similar results from glucometer-based assay disclosed in Example 1 and commercial spectrophotometric assays in longitudinal measurements of patient antibody titers. Patient samples from the TS2 cohort were used to compare our glucometer-based immunoassay (left) to a commercial spectrophotometric assay (Epitope Diagnostics™, right), and strong agreement was observed. We note that differences in detection may be due to divergence in the antigens employed in the assays (our glucometer-based immunoassay uses the SARS-COV-2 spike protein RBD whereas Epitope Diagnostics™ uses the SARS-COV-2 nucleocapsid protein). Not all time points were available to our study, thus resulting in differences in the number of measurements between methods.

### DETAILED DESCRIPTION

Reference will now be made in detail to various exemplary embodiments, examples of which are illustrated in the accompanying drawings. It is to be understood that the following detailed description is provided to give the reader a fuller understanding of certain embodiments, features, and details of aspects of the disclosure, and should not be interpreted as a limitation of the scope of the disclosure.

### Definitions

In order for the present disclosure to be more readily understood, certain terms are first defined below. Additional definitions for the following terms and other terms may be set forth through the specification. If a definition of a term set forth below is inconsistent with a definition in an application or patent that is incorporated by reference, the definition set forth in this application should be used to understand the meaning of the term.

As used in this specification and the appended claims, the singular forms “a,” “an,” and “the” include plural references unless the context clearly dictates otherwise. Thus, for example, a reference to “a method” includes one or more methods, and/or steps of the type described herein and/or which will become apparent to those persons skilled in the art upon reading this disclosure and so forth.

The term “about” is used herein to mean within the typical ranges of tolerances in the art. For example, “about” can be understood as about 2 standard deviations from the mean. According to certain embodiments, when referring to a measurable value such as an amount and the like, “about” is meant to encompass variations of ±20%, ±10%, ±5%, ±1%, ±0.9%, ±0.8%, ±0.7%, ±0.6%, ±0.5%, ±0.4%, ±0.3%, ±0.2% or ±0.1% from the specified value as such variations are appropriate to perform the disclosed methods and/or to make and use the disclosed devices. When “about” is present before a series of numbers or a range, it is understood that “about” can modify each of the numbers in the series or range.

The term “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

The term “antibody” or “antibodies” as used in this disclosure refers to an immunoglobulin or an antigen-binding fragment thereof. As will be understood by those in the art, the immunological binding reagents encompassed by the term “antibody” or “antibodies” extend to all antibodies from all species, and antigen binding fragments thereof and include, unless otherwise specified, polyclonal, monoclonal, monospecific, bispecific, polyspecific, humanized, human, camelised, mouse, non-human primates, single-chain, chimeric, synthetic, recombinant, hybrid, mutated, CDR-grafted, and in vitro generated antibodies. The antibody can include a constant region, or a portion thereof, such as the kappa, lambda, alpha, gamma, delta, epsilon and mu constant region genes. For example, heavy chain constant regions of the various isotypes can be used, including: IgG₁, IgG₂, IgG₃, IgG₄, IgM, IgA₁, IgA₂, IgD, and IgE. By way of example, the light chain constant region can be kappa or lambda.

The term “recombinant antibody fusion protein,” as used herein, refers to a recombinant antibody which comprises a light chain fused to an invertase and/or a heavy chain fused to an invertase such that the recombinant antibody fusion protein retains the antibody binding activity and also has the invertase catalytic activity. In some embodiments, the recombinant antibody fusion protein is a recombinant antibody comprising 2 copies of a light chain fused to an invertase. In some embodiments, the recombinant antibody fusion protein is a recombinant antibody comprising 2 copies of a heavy chain fused to an invertase. In some embodiments, the recombinant antibody fusion protein is a recombinant antibody that bind to a human IgG and has the invertase catalytic activity.

The term “antigenic polypeptide” refers to a polypeptide that is capable of generating an immune response (e.g., the production of antibodies) in a subject. In some embodiments, an antigenic polypeptide is a polypeptide of an infectious pathogen, such as the receptor binding domain (RBD) of the SARS-COV-2. In some embodiments, an antigenic polypeptide is a polypeptide of human origin, such as a polypeptide associated with autoimmune diseases or disorders (e.g., stress proteins).

The terms “antigen-binding domain” and “antigen-binding fragment” refer to a part of an antibody molecule that comprises amino acids responsible for the specific binding between antibody and antigen. For certain antigens, the antigen-binding domain or antigen-binding fragment of an antibody molecule may only bind to a part of the antigen. The part of the antigen that is specifically recognized and bound by the antibody is referred to as the “epitope” or “antigenic determinant.” Antigen-binding domains and antigen-binding fragments include Fab (Fragment antigen-binding); a F(ab′)₂ fragment, a bivalent fragment having two Fab fragments linked by a disulfide bridge at the hinge region; Fv fragment; a single chain Fv fragment (scFv) see e.g., Bird et al. (1988) Science 242:423-426; and Huston et al. (1988) Proc. Natl. Acad. Sci. USA 85:5879-5883); a Fd fragment having the two V_{H} and C_{H}1 domains; dAb (Ward et al., (1989) Nature 341:544-546), and other antibody fragments that retain antigen-binding function. The Fab fragment has V_{H}-C_{H} and V_{L}-C_{L} domains covalently linked by a disulfide bond between the constant regions. The Fᵥ fragment is smaller and has V_{H} and V_{L} domains non-covalently linked. To overcome the tendency of non-covalently linked domains to dissociate, a scFv can be constructed. The scFv contains a flexible polypeptide that links (1) the C-terminus of V_{H} to the N-terminus of V_{L}, or (2) the C-terminus of V_{L} to the N-terminus of V_{H}. These antibody fragments are obtained using conventional techniques known to those with skill in the art, and the fragments are evaluated for function in the same manner as are intact antibodies.

The term “at least” prior to a number or series of numbers (e.g., “at least two”) is understood to include the number adjacent to the term “at least,” and all subsequent numbers or integers that could logically be included, as clear from context. When “at least” is present before a series of numbers or a range, it is understood that “at least” can modify each of the numbers in the series or range.

As used herein, the term “binds” or “binding” refers to the interaction between an antibody, or an antigen-binding fragment, and an antigen, or an antigenic polypeptide or fragment. In the context of pathogen detection, the term “binds” or “binding” refers to a state in which a first chemical structure (e.g., a pathogenic particle or polypeptide) is sufficiently associated a second chemical structure (e.g., an antibody or an antigen-binding fragment that binds to the pathogenic particle or polypeptide) such that the association between the first and second chemical structures can be detected.

As used herein, the term “detecting,” “detect,” or “detection” refers to an act of determining the existence or presence of one or more target analytes (e.g., antibody to an antigenic polypeptide, such as the receptor binding domain (RBD) of the SARS-COV-2 spike protein) in a sample.

The term “diagnosing” or “diagnosis” as used herein refers to the use of information (e.g., antibody binding or data from tests on biological samples, signs and symptoms, physical exam findings, cognitive performance results, etc.) to anticipate the most likely outcomes, timeframes, and/or response to a particular treatment for a given disease, disorder, or condition, based on comparisons with a plurality of individuals sharing common nucleotide sequences, symptoms, signs, family histories, or other data relevant to consideration of a patient's health status.

The term “fragment,” as used herein in reference to a protein, refers to a polypeptide that has an amino-terminal and/or carboxy-terminal deletion as compared to the native, full-length protein. Fragments typically are at least 4 amino acids long, preferably at least 20 amino acids long, usually at least 50 amino acids long or longer, and may span the portion of the full-length protein required for intermolecular binding with its various ligands and/or substrates.

The term “identity,” as known in the art, is a relationship between two or more polypeptide sequences or two or more polynucleotide sequences, as determined by comparing the sequences. In the art, “identity” also means the degree of sequence relatedness between polypeptide or polynucleotide sequences, as determined by the match between strings of such sequences. “Identity” and “similarity” can be readily calculated by known methods, including, but not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., cds., Humana Press, New Jersey, 1994; Sequence Analysis in Molecular Biology, von Heinje, G., Academic Press, 1987; and Sequence Analysis Primer, Gribskov, M. and Devereux, J., cds., M Stockton Press, New York, 1991; and Carillo, H., and Lipman, D., Siam J. Applied Math., 48:1073 (1988). Typical methods to determine identity are designed to give the largest match between the sequences tested. Methods to determine identity and similarity are codified in publicly available computer programs. Typical computer program methods to determine identity and similarity between two sequences include, but are not limited to, the GCG program package (Devereux, J., et al., Nucleic Acids Research 12 (1): 387 (1984)), BLASTP, BLASTN, and FASTA (Atschul, S. F. et al., J. Molec. Biol. 215:403-410 (1990). The BLAST X program is publicly available from NCBI and other sources (BLAST Manual, Altschul, S., et al., NCBINLM NIH Bethesda, Md. 20894: Altschul, S., et al., J. Mol. Biol. 215:403-410 (1990). The well-known Smith Waterman algorithm may also be used to determine identity.

The term “immunoassay” refers to any assay that uses at least one specific antibody for the detection and/or quantification of an antigen. Immunoassays include, but not limited to, rapid strip tests, Western blots, enzyme-linked immunosorbent assays (ELISAs), radio-immunoassays, and immunofluorescence assays and any other antigen-antibody reactions including, for example, “flocculation” (i.e., a colloidal suspension produced upon the formation of antigen-antibody complexes), “agglutination” (i.e., clumping of cells or other substances upon exposure to antibody), “particle agglutination” (i.e., clumping of particles coated with antigen in the presence of antibody or the clumping of particles coated with antibody in the presence of antigen), “complement fixation” (i.e., the use of complement in an antibody-antigen reaction method), and other methods commonly used in serology, immunology, immunocytochemistry, immunohistochemistry, and related fields.

The term “in need thereof” means that the subject has been identified or suspected as having a need for the particular method or treatment. In some embodiments, the identification can be by any means of diagnosis or observation. In any of the methods described herein, the subject can be in need thereof. In some embodiments, the subject in need thereof is a human suspected of having a pathogenic infection, such as an infection caused by SARS-COV-2. In some embodiments, the subject in need thereof is a human diagnosed with a pathogenic infection, such as an infection caused by SARS-COV-2. In some embodiments, the subject in need thereof is a human seeking treatment for a pathogenic infection, such as an infection caused by SARS-COV-2. In some embodiments, the subject in need thereof is a human undergoing treatment for a pathogenic infection, such as an infection caused by SARS-COV-2.

As used herein, the term “in some embodiments” refers to embodiments of all aspects of the disclosure, unless the context clearly indicates otherwise.

Invertase is an enzyme that catalyzes the hydrolysis of sucrose into fructose and glucose. Thus, the term “invertase catalytic activity” as used herein refers to the catalytic activity of converting at least one substrate, such as sucrose, into at least one detectable product, such as fructose and/or glucose.

The term “kit” refers to a combination of reagents and/or apparatus, which facilitates sample analysis. In some embodiments, a kit may further include one or more apparatus to facilitate sample harvesting. In some embodiments, a kit may further include one or more reagents for sample processing. In some embodiments, a kit may further include one or more written instructions.

The term “pathogen” refers to anything that can produce a disease, condition, or disorder in a subject. In some embodiments, a pathogen includes an infectious microorganism or agent, such as a bacterium, virus, viroid, protozoan, prion, or fungus.

The term “point-of-care” refers to the point in time when healthcare products and services are delivered to patients at the time of care. A “point-of-care” test or method, also called bedside testing, refers to a medical diagnostic test or method that can be performed at or near the point of care—that is, at the time and place of patient care. In the diagnostic setting, this means that the diagnosis occurs at the time and place where the test is administered to the patient. For example, with a point-of-care embodiment in the context of the methods disclosed herein, a sample may be obtained from the patient and tested using the methods and/or devices disclosed herein without having to send the sample to a different location for testing. In this way, the result of the test can be provided to the patient at the same location where the test was administered, typically within minutes, or in even less time. A “point-of-care” testing contrasts with testing that is wholly or mostly confined to a medical laboratory and often entails collecting a sample and sending the sample away from the point of care location and then waiting hours or days to learn the results, during which time care must be withheld or administered without the desired diagnostic result.

The term “protein” or “polypeptide” refers to a polymer of amino acids, peptide nucleic acids (PNAs) or mimetics, of no specific length and to all fragments, isoforms, variants, derivatives and modifications (glycosylation, phosphorylation, post-translational modifications, etc.) thereof.

The term “sample” is used herein in the broadest sense and can be obtained from any source in the body. A sample can encompass fluids, solids and/or tissues. In some embodiments, a sample can include one or more of the following fluids: oral fluid, nasal fluid, or car drainage. A sample can also include other fluids, such as serous fluid, urine, saliva, tears, blood, plasma, and serum. In some embodiments, the sample is an oral fluid, nasal fluid, saliva, blood, plasma, or serum. Although the sample is typically taken from a human subject, the assays can be used to detect antibody in samples from any mammal, such as dogs, cats, sheep, cattle, and pigs, etc. The sample may be pretreated as necessary by dilution in an appropriate buffer solution or concentrated, if desired. Any of known standard aqueous buffer solutions, such as phosphate, Tris, or the like, at or near physiological pH can be used and the term sample is intended to include pre-treated samples as well as acute samples.

The terms “subject” refers to an animal, such as a mammalian species (e.g., human) or avian (e.g., bird) species. More specifically, a subject can be a vertebrate, e.g., a mammal such as a mouse, a primate, a simian, or a human. Animals include farm animals (e.g., production cattle, dairy cattle, poultry, horses, pigs, and the like), sport animals, and companion animals (e.g., pets or support animals). A subject can be a healthy individual, an individual that has or is suspected of having a disease or a predisposition to the disease, or an individual that needs therapy or suspected of needing therapy. The terms “individual” or “patient” are intended to be interchangeable with “subject.” For example, a subject can be an individual who has been diagnosed with having a pathogenic infection, is going to receive a therapy for a pathogenic infection, and/or has received at least one therapy for a pathogenic infection.

The term “solid support” refers to any rigid or semi-rigid support onto which molecules (e.g., nucleic acids, polypeptides, mimetics) may be bound. Examples of solid supports include, but not limited to, membranes, filters, chips, slides, wafers, fibers, magnetic, or nonmagnetic beads, gels, capillaries or other tubing, plates, polymers, and microparticles with a variety of surface forms including wells, trenches, pins, channels, and pores. In some embodiments, the solid support is a strip, bead, plate, or slide.

The term “treat,” “treated,” or “treating” refers to therapeutic treatment and/or prophylactic or preventative measures wherein the object is to prevent or slow down (lessen) an undesired physiological condition, disorder, or disease, or obtain beneficial or desired clinical results. For purposes of the embodiments described herein, beneficial or desired clinical results include, but are not limited to, alleviation of symptoms; diminishment of extent of condition, disorder or disease; stabilized (i.e., not worsening) state of condition, disorder or disease; delay in onset or slowing of condition, disorder or disease progression; amelioration of the condition, disorder or disease state or remission (whether partial or total), whether detectable or undetectable; an amelioration of at least one measurable physical parameter, not necessarily discernible by the patient; or enhancement or improvement of condition, disorder or disease. Treatment can also include eliciting a clinically significant response without excessive levels of side effects. Treatment also includes prolonging survival as compared to expected survival if not receiving treatment.

As used herein, the term “therapeutic” means an agent utilized to treat, combat, ameliorate, prevent, or improve an unwanted condition or disease of a patient.

A “therapeutically effective amount” of a treatment is a predetermined amount calculated to achieve the desired effect, i.e., to treat, combat, ameliorate, prevent, or improve one or more symptoms of a viral infection. The activity contemplated by the present methods includes both medical therapeutic and/or prophylactic treatment, as appropriate. The specific dose of a compound administered according to the present disclosure to obtain therapeutic and/or prophylactic effects will, of course, be determined by the circumstances surrounding the case, including, for example, the compound administered, the route of administration, and the condition being treated. It will be understood that the effective amount administered will be determined by the physician in the light of the relevant circumstances including the condition to be treated, the choice of compound to be administered, and the chosen route of administration, and therefore the above dosage ranges are not intended to limit the scope of the present disclosure in any way. A therapeutically effective amount of compounds, such as antibiotics, antivirals or antifungals, for treating a pathogenic infection according to the disclosure is typically an amount such that when it is administered in a physiologically tolerable excipient composition, it is sufficient to achieve an effective systemic concentration or local concentration in the tissue.

### Recombinant Antibody Fusion Proteins

Disclosed herein are recombinant antibody fusion proteins (i.e., recombinant antibodies comprising anti-human IgG-invertase fusion proteins) that can be used as reporter enzymes and incorporated into a quick, reliable, and affordable immunoassay for antibody detection. The recombinant antibody fusion proteins of the disclosure are invertase-based without the issues of inefficient and inconsistent coupling of invertase to detection molecules, which has previously hindered the development of invertase-based detection systems. The recombinant antibody fusion proteins of the disclosure fully preserve the antibody binding activity (e.g., human IgG binding activity) and the invertase catalytic activity (e.g., hydrolysis of sucrose into fructose and glucose) relative to unmodified proteins.

The recombinant antibody fusion proteins of the disclosure are based on genetic fusions of an invertase to an anti-human IgG antibody fragment via a peptide linker. Thus, in some embodiments, provided herein is a polypeptide comprising an anti-human IgG antibody fragment fused to an invertase, and a linker positioned between the anti-human IgG antibody fragment and the invertase. In some embodiments, the anti-human IgG antibody fragment is a light chain of an anti-human IgG antibody. In some embodiments, the anti-human IgG antibody fragment is a heavy chain of an anti-human IgG antibody.

Any anti-human IgG antibody fragment can be used. An anti-human IgG antibody fragment can be identified and isolated from, for instance, a hybridoma clone producing a mouse monoclonal antibody to human IgG using any methods known in the art. Exemplary hybridoma clones producing mouse monoclonal antibodies to human IgG include, but not limited to clone HP6017 and clone IG266. For example, as illustrated in Example 1 below, total RNA can be isolated from the hybridoma cells and then reverse-transcribed into cDNA using any methods known in the art. Antibody fragments of heavy chain and light chain can then be amplified using procedures known in the art, such as rapid amplification of cDNA ends (RACE), and cloned into a standard cloning vector separately. Using hybridoma clone HP6017 as an example, the heavy chain and light chain can be separately cloned into a standard expression vector and produced to formulate the recombinant antibody HP6017 as mouse IgG 2a kappa isotype to match the parent clone.

The heavy chain of the recombinant antibody HP6017 comprises the following amino acid sequence:

(SEQ ID NO: 2; bold: Ab V_{H}; italic: mouse
IgG2a C_{H}1, C_{H}2, and C_{H}3)
QVQLQQPGAELVKPGASVKMSCKASGYTFTNYWINWVKQRPGQGL
EWIGDIYPGGGITNYNEKFKTKATLTLDTSSSTVYMQLSSLTSED
SAVYYCSRSYGKYFDYWGQGTTLIVSSAKTTAPSVYPLAPVCGDT
TGSSVTLGCLVKGYFPEPVTLTWNSGSLSSGVHTFPAVLQSDLYT
LSSSVTVTSSTWPSQSITCNVAHPASSTKVDKKIEPRGPTIKPCP
PCKCPAPNLLGGPSVFIFPPKIKDVLMISLSPIVTCVVVDVSEDD
PDVQISWFVNNVEVHTAQTQTHREDYNSTLRVVSALPIQHQDWMS
GKEFKCKVNNKDLPAPIERTISKPKGSVRAPQVYVLPPPEEEMTK
KQVTLTCMVTDFMPEDIYVEWTNNGKTELNYKNTEPVLDSDGSYF
MYSKLRVEKKNWVERNSYSCSVVHEGLHNHHTTKSFSRTPGK.

It is encoded by the following nucleic acid sequence:

(SEQ ID NO: 1; bold: Ab V_{H}; italic: mouse
IgG2a C_{H}1, C_{H}2, and C_{H}3)
caggtccaactgcagcagcctggggctgagcttgtgaagcctggg
gcttcagtgaagatgtcctgcaaggctagcggctacaccttcacc
aactactggataaactgggtgaagcagaggcctggacaaggtcta
gagtggattggagatatttatcctggtggaggtattactaattac
aatgagaagttcaagaccaaggccacactgactctggacacatcc
tcgtcgacagtctatatgcaactcagcagcctgacatctgaagac
tctgcggtatactattgttcaagatcctatggtaaatatttcgac
tactggggccaaggcaccactctcatagtctcctcagccaaaaca
acggcgccatcggtctatccactggcccctgtgtgtggagataca
actggctcgtcggtgactctaggatgcctggtcaagggttatttc
cctgagccagtgaccttgacctggaactctggttccctgtccagt
ggtgtgcacaccttcccagctgtcctccagtctgacctctacacc
ctcagcagctcagtgactgtaacctcgagcacctggcccagccag
tccatcacctgcaatgtggcccacccggcaagcagcaccaaggtg
gacaagaaaattgagcccagagggcccacaatcaagccctgtcct
ccatgcaaatgcccagcacctaacctcttgggtggaccatccgtc
ttcatcttccctccaaagatcaaggatgtactcatgatctccctg
agccccatagtcacatgtgtggtggtggatgtgagcgaggatgac
ccagatgtccagatcagctggtttgtgaacaacgtggaagtacac
acagctcagacacaaacccatagagaggattacaacagtactctc
cgggtggtcagtgccctccccatccagcaccaggactggatgagt
ggcaaggagttcaaatgcaaggtcaacaacaaagacctcccagcg
cccatcgagagaaccatctcaaaacccaaagggtcagtaagagct
ccacaggtatatgtcttgcctccaccagaagaagagatgactaag
aaacaggtcactctgacctgcatggtcacagacttcatgcctgaa
gacatttacgtggagtggaccaacaacgggaaaacagagctaaac
tacaagaacactgaaccagtcctggactctgatggttcttacttc
atgtacagcaagctgagagtggaaaagaagaactgggtggaaaga
aatagctactcctgttcagtggtccacgagggtctgcacaatcac
cacacgactaagagcttctcccggactccgggtaaa.

The light chain of the recombinant antibody HP6017 comprises the following amino acid sequence:

(SEQ ID NO: 4; bold: Ab V_{L}; italic: mouse
Kappa C_{L})
DIKMTQSPSSMYASVGERVTITCKASQDIKSYLTWYQKKPWKSPR
TLIFYSTRLADGVPSRFSGSGSGQDFSLTISSLESDDTATYYCLQ
HDESPFTFGGGTKLEIKRADAAPTVSIFPPSSEQLTSGGASVVCF
LNNFYPKDINVKWKIDGSERQNGVLNSWTDQDSKDSTYSMSSTLT
LTKDEYERHNSYTCEATHKTSTSPIVKSFNRNEC.

It is encoded by the following nucleic acid sequence:

(SEQ ID NO: 3; bold: Ab V_{L}; italic: mouse
Kappa C_{L})
gatatcaagatgacccagtccccatcctccatgtatgcatcggtg
ggagagagagtcactatcacgtgcaaggcgagtcaggacattaaa
agctatttaacctggtaccagaagaagccatggaaatctcctagg
accctgatcttctattcaacaaggttggcagatggggtcccatca
agattcagtggcagtggatctgggcaagatttttctctaaccatc
agcagcctggagtctgacgatacagcaacttattactgtctccag
catgatgagagcccgttcacgttcggaggagggaccaagctggaa
ataaaacgggctgatgctgcaccaactgtatccatcttcccacca
tccagtgagcagttaacatctggaggtgcctcagtcgtgtgcttc
ttgaacaacttctaccccaaagacatcaatgtcaagtggaagatt
gatggcagtgaacgacaaaatggcgtcctgaacagttggactgat
caggacagcaaagacagcacctacagcatgagcagcaccctcacg
ttgaccaaggacgagtatgaacgacataacagctatacctgtgag
gccactcacaagacatcaacttcacccattgtcaagagcttcaac
aggaatgagtgt.

Thus, in some embodiments, the anti-human IgG antibody fragment comprised in the recombinant antibody fusion proteins of the disclosure comprise an amino acid sequence having at least about 70%, 75%, 80%, 85, 90%, 95%, 96%, 97%, 98% or 99% sequence identity to the amino acid sequence of SEQ ID NO: 2. In some embodiments, the anti-human IgG antibody fragment comprised in the recombinant antibody fusion proteins of the disclosure comprises the amino acid sequence of SEQ ID NO: 2. In some embodiments, the anti-human IgG antibody fragment comprised in the recombinant antibody fusion proteins of the disclosure comprises an amino acid sequence having at least about 70%, 75%, 80%, 85, 90%, 95%, 96%, 97%, 98% or 99% sequence identity to the amino acid sequence of SEQ ID NO: 4. In some embodiments, the anti-human IgG antibody fragment comprised in the recombinant antibody fusion proteins of the disclosure comprises the amino acid sequence of SEQ ID NO: 4. In some embodiments, the anti-human IgG antibody fragment comprised in the recombinant antibody fusion proteins of the disclosure is encoded by a nucleic acid molecule comprising at least about 70%, 75%, 80%, 85, 90%, 95%, 96%, 97%, 98% or 99% sequence identity to the nucleic acid sequence of SEQ ID NO: 1 or SEQ ID NO: 3. In some embodiments, the anti-human IgG antibody fragment comprised in the recombinant antibody fusion proteins of the disclosure is encoded by a nucleic acid molecule comprising the nucleic acid sequence of SEQ ID NO: 1 or SEQ ID NO: 3.

Any invertase can be used to form the genetic fusion with the anti-human IgG antibody fragment disclosed herein. An exemplary source of invertase is yeast, such as Saccharomyces cerevisiae (e.g., UniProt Accession No. P00724-2). Another exemplary source of invertase is bacteria, such as Zymomonas mobilis (GenBank Accession No. BAA04476). For instance, Saccharomyces cerevisiae invertase with UniProt Accession No. P00724-2 comprises the following amino acid sequence:

(SEQ ID NO: 6)
MTNETSDRPLVHFTPNKGWMNDPNGLWYDEKDAKWHLYFQYNPND
TVWGTPLFWGHATSDDLTNWEDQPIAIAPKRNDSGAFSGSMVVDY
NNTSGFFNDTIDPRQRCVAIWTYNTPESEEQYISYSLDGGYTFTE
YQKNPVLAANSTQFRDPKVFWYEPSQKWIMTAAKSQDYKIEIYSS
DDLKSWKLESAFANEGFLGYQYECPGLIEVPTEQDPSKSYWVMFI
SINPGAPAGGSFNQYFVGSFNGTHFEAFDNQSRVVDFGKDYYALQ
TFFNTDPTYGSALGIAWASNWEYSAFVPTNPWRSSMSLVRKFSLN
TEYQANPETELINLKAEPILNISNAGPWSRFATNTTLTKANSYNV
DLSNSTGTLEFELVYAVNTTQTISKSVFADLSLWFKGLEDPEEYL
RMGFEVSASSFFLDRGNSKVKFVKENPYFTNRMSVNNQPFKSEND
LSYYKVYGLLDQNILELYFNDGDVVSTNTYFMTTGNALGSVNMTT
GVDNLFYIDKFQVREVK.

It is encoded by the following nucleic acid sequence:

(SEQ ID NO: 5)
atgacaaacgaaactagcgatagacctttggtccacttcacaccc
aacaagggctggatgaatgacccaaatgggttgtggtacgatgaa
aaagatgccaaatggcatctgtactttcaatacaacccaaatgac
accgtatggggtacgccattgttttggggccatgctacttccgat
gatttgactaattgggaagatcaacccattgctatcgctcccaag
cgtaacgattcaggtgctttctctggctccatggtggttgattac
aacaacacgagtgggtttttcaatgatactattgatccaagacaa
agatgcgttgcgatttggacttataacactcctgaaagtgaagag
caatacattagctattctcttgatggtggttacacttttactgaa
taccaaaagaaccctgttttagctgccaactccactcaattcaga
gatccaaaggtgttctggtatgaaccttctcaaaaatggattatg
acggctgccaaatcacaagactacaaaattgaaatttactcctct
gatgacttgaagtcctggaagctagaatctgcatttgccaatgaa
ggtttcttaggctaccaatacgaatgtccaggtttgattgaagtc
ccaactgagcaagatccttccaaatcttattgggtcatgtttatt
tctatcaacccaggtgcacctgctggcggttccttcaaccaatat
tttgttgggtccttcaatggtactcattttgaagcgtttgacaat
caatctagagtggtagattttggtaaggactactatgccttgcaa
actttcttcaacactgacccaacctacggttcagcattaggtatt
gcctgggcttcaaactgggagtacagtgcctttgtcccaactaac
ccatggagatcatccatgtctttggtccgcaagttttctttgaac
actgaatatcaagctaatccagagactgaattgatcaatttgaaa
gccgaaccaatattgaacattagtaatgctggtccctggtctcgt
tttgctactaacacaactctaactaaggccaattcttacaatgtc
gatttgagcaactcgactggtaccctagagtttgagttggtttac
gctgttaacaccacacaaaccatatccaaatccgtctttgccgac
ttatcactttggttcaagggtttagaagatcctgaagaatatttg
agaatgggttttgaagtcagtgcttcttccttctttttggaccgt
ggtaactctaaggtcaagtttgtcaaggagaacccatatttcaca
aacagaatgtctgtcaacaaccaaccattcaagtctgagaacgac
ctaagttactataaagtgtacggcctactggatcaaaacatcttg
gaattgtacttcaacgatggagatgtggtttctacaaatacctac
ttcatgaccaccggtaacgctctaggatctgtgaacatgaccact
ggtgtcgataatttgttctacattgacaagttccaagtaagggaa
gtaaaa.

Thus, in some embodiments, the invertase comprised in the recombinant antibody fusion proteins of the disclosure comprise an amino acid sequence having at least about 70%, 75%, 80%, 85, 90%, 95%, 96%, 97%, 98% or 99% sequence identity to the amino acid sequence of SEQ ID NO: 6. In some embodiments, the invertase comprised in the recombinant antibody fusion proteins of the disclosure comprises the amino acid sequence of SEQ ID NO: 6. In some embodiments, the invertase comprised in the recombinant antibody fusion proteins of the disclosure is encoded by a nucleic acid molecule comprising at least about 70%, 75%, 80%, 85, 90%, 95%, 96%, 97%, 98% or 99% sequence identity to the nucleic acid sequence of SEQ ID NO: 5. In some embodiments, the invertase comprised in the recombinant antibody fusion proteins of the disclosure is encoded by a nucleic acid molecule comprising the nucleic acid sequence of SEQ ID NO: 5.

The genetic fusion between an anti-human IgG antibody fragment and an invertase comprised in the recombinant antibody fusion proteins of the disclosure is linked by a linker so that the linker is positioned between the anti-human IgG antibody fragment and the invertase. Any type of linker or linker peptide can be used. The term “linker” or “linker peptide” is used interchangeable herein.

Linkers of any length can be used to form a genetic fusion between an anti-human IgG antibody fragment and an invertase. In some embodiments, the linker is from about 1 to about 30, about 2 to about 30, about 3 to about 30, about 4 to about 30, about 5 to about 30, about 6 to about 30, about 7 to about 30, about 8 to about 30, about 9 to about 30, about 10 to about 30, about 11 to about 30, about 12 to about 30, about 13 to about 30, about 14 to about 30, about 15 to about 30, about 16 to about 30, about 17 to about 30, about 18 to about 30, about 19 to about 30, about 20 to about 30, about 21 to about 30, about 22 to about 30, about 23 to about 30, about 24 to about 30, or about 25 to about 30 natural or non-natural amino acids in length.

In some embodiments, the linker is about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 natural or non-natural amino acids in length. In some embodiments, the linker is about 10 natural or non-natural amino acids in length. In some embodiments, the linker is about 15 natural or non-natural amino acids in length. In some embodiments, the linker is about 17 natural or non-natural amino acids in length. In some embodiments, the linker is about 20 natural or non-natural amino acids in length. In some embodiments, the linker is about 25 natural or non-natural amino acids in length. In some embodiments, the linker is about 27 natural or non-natural amino acids in length. In some embodiments, the linker is about 30 natural or non-natural amino acids in length.

A non-limiting example of a linker may comprise the amino acid sequence of GGGGSGGGGSGGGGS (SEQ ID NO: 8) encoded by the nucleic acid sequence of ggtggtggaggttcagggggtggaggtagcggcggagggggatcc (SEQ ID NO: 7). Another non-limiting example of a linker may comprise the amino acid sequence of ASGGGGSGGGGSGGGGS (SEQ ID NO: 10) encoded by the nucleic acid sequence of gctagcggtggtggaggttcaggcgggggcggtagcgg cggtgggggatcc (SEQ ID NO: 9). A yet another non-limiting example of a linker may comprise the amino acid sequence of GGGGSGGGGSGGGGSGGGGSGGGGS (SEQ ID NO: 12) encoded by the nucleic acid sequence of ggtggtggaggttcagggggtggaggatctggcggagggggttcaggcgggggcg gtagcggcggtgggggatcc (SEQ ID NO: 11). A further another non-limiting example of a linker may comprise the amino acid sequence of ASGGGGSGGGGSGGGGSGGGGSGGGGS (SEQ ID NO: 14) encoded by the nucleic acid sequence of gctagcggtggtggaggttcagggggtggaggatctggcggagggggt tcaggcgggggcggtagcggcggtgggggatcc (SEQ ID NO: 13).

Accordingly, in some embodiments, the linker used to form the genetic fusion between an anti-human IgG antibody fragment and an invertase comprised in the recombinant antibody fusion proteins of the disclosure comprises an amino acid sequence having at least about 70%, 75%, 80%, 85%, 90%, 95%, 96%, 97%, 98% or 99% sequence identity to SEQ ID NO: 8, SEQ ID NO: 10, SEQ ID NO: 12 or SEQ ID NO: 14. In some embodiments, the linker comprises the amino acid sequence of SEQ ID NO: 8, SEQ ID NO: 10, SEQ ID NO: 12 or SEQ ID NO: 14. In some embodiments, the linker used to form the genetic fusion between an anti-human IgG antibody fragment and an invertase comprised in the recombinant antibody fusion proteins of the disclosure is encoded by a nucleic acid molecule comprising at least about 70%, 75%, 80%, 85%, 90%, 95%, 96%, 97%, 98% or 99% sequence identity to SEQ ID NO: 7, SEQ ID NO: 9, SEQ ID NO: 11 or SEQ ID NO: 13. In some embodiments, the linker is encoded by a nucleic acid molecule comprising the nucleic acid sequence of SEQ ID NO: 7, SEQ ID NO: 9, SEQ ID NO: 11 or SEQ ID NO: 13.

In some embodiments, the genetic fusion of an anti-human IgG antibody fragment and an invertase comprised in the recombinant antibody fusion proteins of the disclosure further comprises a signal peptide positioned at the N-terminus of the genetic fusion polypeptide. A “signal peptide” may be from time to time refers to a “leader sequence” and thus, the terms “signal peptide” and “leader sequence” are used interchangeably herein and refer to a peptide that can be linked at the N-terminus of a protein set forth herein. Signal peptides typically direct localization of a protein. Signal peptides used herein preferably facilitate secretion of the protein from the cell in which it is produced. Signal peptides are often cleaved from the remainder of the protein, often referred to as the mature protein, upon secretion from the cell. Signal peptides, when present, are linked at the N terminus of the protein.

A non-limiting example of the signal peptide is the amino acid sequence of MGWSCIILFLVATATGVHS (SEQ ID NO: 16) encoded by the nucleic acid sequence of atgggatggagctgtatcatcctctttttggtagcaacagctacaggtgtccactcc (SEQ ID NO: 15). Another non-limiting example of the signal peptide is the amino acid sequence of MRAPAQFFGILLLWFPGIRC (SEQ ID NO: 18) encoded by the nucleic acid sequence of atgagggcccctgctcagttttttgggatcttgttgctctggtttccaggtatcagatgt (SEQ ID NO: 17). A further non-limiting example of the signal peptide is the amino acid sequence of MRVPAQLLGLLLLWLPGARC (SEQ ID NO: 20) encoded by the nucleic acid sequence of atgagggtccccgctcagctcctggggctcctgctgctctggctcccaggtgcacgatgt (SEQ ID NO: 19).

Thus, in some embodiments, when the signal peptide is present, the signal peptide comprises an amino acid sequence having at least about 70%, 75%, 80%, 85%, 90%, 95%, 96%, 97%, 98% or 99% sequence identity to SEQ ID NO: 16, SEQ ID NO: 18, or SEQ ID NO: 20. In some embodiments, when the signal peptide is present, the signal peptide comprises the amino acid sequence of SEQ ID NO: 16, SEQ ID NO: 18, or SEQ ID NO: 20. In some embodiments, when the signal peptide is present, the signal peptide is encoded by a nucleic acid molecule comprising at least about 70%, 75%, 80%, 85%, 90%, 95%, 96%, 97%, 98% or 99% sequence identity to SEQ ID NO: 15, SEQ ID NO: 17, or SEQ ID NO: 19. In some embodiments, when the signal peptide is present, the signal peptide is encoded by a nucleic acid molecule comprising the nucleic acid sequence of SEQ ID NO: 15, SEQ ID NO: 17, or SEQ ID NO: 19.

The genetic fusion of an anti-human IgG antibody fragment and an invertase comprised in the recombinant antibody fusion proteins of the disclosure can be a combination of any of the anti-human IgG antibody fragments, invertases, linkers, and/or signal peptides described herein. For example, in some embodiments, the heavy chain of the recombinant antibody HP6017 of SEQ ID NO: 2 may be fused to the invertase of SEQ ID NO: 6 via a linker of SEQ ID NO: 10 and a signal peptide of SEQ ID NO: 16 may be added to the N-terminus of SEQ ID NO: 2, leading to the genetic fusion of SEQ ID NO: 26 (which comprises the heavy chain polypeptide of the antibody HP6017 fused to an invertase; referred to as “Ab+Inv HC17” in Example 1). In other embodiments, the heavy chain of SEQ ID NO: 2 may be fused to the invertase of SEQ ID NO: 6 via a linker of SEQ ID NO: 14 and a signal peptide of SEQ ID NO: 16 may be added to the N-terminus of SEQ ID NO: 2, leading to the genetic fusion of SEQ ID NO: 28 (which comprises the heavy chain polypeptide of the antibody HP6017 fused to an invertase; referred to as “Ab+Inv HC27” in Example 1). Alternatively, in some embodiments, the light chain of the recombinant antibody HP6017 of SEQ ID NO: 4 may be fused to the invertase of SEQ ID NO: 6 via a linker of SEQ ID NO: 8 and a signal peptide of SEQ ID NO: 18 may be added to the N-terminus of SEQ ID NO: 4, leading to the genetic fusion of SEQ ID NO: 30 (which comprises the light chain polypeptide of the antibody HP6017 fused to an invertase; referred to as “Ab+Inv LC15” in Example 1). In other embodiments, the light chain of SEQ ID NO: 4 may be fused to the invertase of SEQ ID NO: 6 via a linker of SEQ ID NO: 12 and a signal peptide of SEQ ID NO: 18 may be added to the N-terminus of SEQ ID NO: 4, leading to the genetic fusion of SEQ ID NO: 32 (which comprises the light chain polypeptide of the antibody HP6017 fused to an invertase; referred to as “Ab+Inv LC25” in Example 1).

Accordingly, also provided herein are polypeptides comprising the genetic fusion of an anti-human IgG antibody fragment and an invertase, wherein the anti-human IgG antibody fragment is fused to the invertase via a linker and a signal peptide is added to the N-terminus of the anti-human IgG antibody fragment. In some embodiments, the polypeptide comprises an amino acid sequence having at least about 70%, 75%, 80%, 85%, 90%, 95%, 96%, 97%, 98% or 99% sequence identity to SEQ ID NO: 26, SEQ ID NO: 28, SEQ ID NO: 30, or SEQ ID NO: 32. In some embodiments, the polypeptide comprises the amino acid sequence of SEQ ID NO: 26, SEQ ID NO: 28, SEQ ID NO: 30, or SEQ ID NO: 32. In some embodiments, the polypeptide is encoded by a nucleic acid molecule comprising at least about 70%, 75%, 80%, 85%, 90%, 95%, 96%, 97%, 98% or 99% sequence identity to SEQ ID NO: 25, SEQ ID NO: 27, SEQ ID NO: 29, or SEQ ID NO: 31. In some embodiments, the polypeptide is encoded by a nucleic acid molecule comprising the nucleic acid sequence of SEQ ID NO: 25, SEQ ID NO: 27, SEQ ID NO: 29, or SEQ ID NO: 31.

Any of the genetic fusions of an anti-human IgG antibody fragment and an invertase described herein can be used to generate a recombinant antibody fusion protein of the disclosure. Because an antibody unit generally consists of four polypeptide chains, two identical heavy chains and two identical light chains connected by disulfide bonds, the recombinant antibody fusion protein of the disclosure comprises 2 identical copies of any of the genetic fusions of an anti-human IgG antibody fragment and an invertase described herein in some embodiments. For instance, a recombinant antibody fusion protein of the disclosure may comprise 2 identical copies of a genetic fusion comprising the heavy chain of the recombinant antibody HP6017 of SEQ ID NO: 2 and, in such cases, the recombinant antibody fusion protein may further comprise 2 identical copies of the light chain of the recombinant antibody HP6017 of SEQ ID NO: 4. Conversely, a recombinant antibody fusion protein of the disclosure may comprise 2 identical copies of a genetic fusion comprising the light chain of the recombinant antibody HP6017 of SEQ ID NO: 4 and, in such cases, the recombinant antibody fusion protein may further comprise 2 identical copies of the heavy chain of the recombinant antibody HP6017 of SEQ ID NO: 2.

Accordingly, in some embodiments, the recombinant antibody fusion protein of the disclosure comprises 2 copies of a genetic fusion of an anti-human IgG antibody fragment and an invertase comprising at least about 70%, 75%, 80%, 85%, 90%, 95%, 96%, 97%, 98%, 99%, or 100% sequence identity to SEQ ID NO: 26 or SEQ ID NO: 28 and 2 copies of an anti-human IgG antibody fragment comprising at least about 70%, 75%, 80%, 85%, 90%, 95%, 96%, 97%, 98%, 99%, or 100% sequence identity to SEQ ID NO: 24. In some embodiments, the recombinant antibody fusion protein of the disclosure comprises 2 copies of a genetic fusion comprising the amino acid sequence of SEQ ID NO: 26 and 2 copies of an anti-human IgG antibody fragment comprising the amino acid sequence of SEQ ID NO: 24. In some embodiments, the recombinant antibody fusion protein of the disclosure comprises 2 copies of a genetic fusion comprising the amino acid sequence of SEQ ID NO: 28 and 2 copies of an anti-human IgG antibody fragment comprising the amino acid sequence of SEQ ID NO: 24. In other embodiments, the recombinant antibody fusion protein of the disclosure comprises 2 copies of a genetic fusion of an anti-human IgG antibody fragment and an invertase comprising at least about 70%, 75%, 80%, 85%, 90%, 95%, 96%, 97%, 98%, 99%, or 100% sequence identity to SEQ ID NO: 30 or SEQ ID NO: 32 and 2 copies of an anti-human IgG antibody fragment comprising at least about 70%, 75%, 80%, 85%, 90%, 95%, 96%, 97%, 98%, 99%, or 100% sequence identity to SEQ ID NO: 22. In some embodiments, the recombinant antibody fusion protein of the disclosure comprises 2 copies of a genetic fusion comprising the amino acid sequence of SEQ ID NO: 30 and 2 copies of an anti-human IgG antibody fragment comprising the amino acid sequence of SEQ ID NO: 22. In some embodiments, the recombinant antibody fusion protein of the disclosure comprises 2 copies of a genetic fusion comprising the amino acid sequence of SEQ ID NO: 32 and 2 copies of an anti-human IgG antibody fragment comprising the amino acid sequence of SEQ ID NO: 22.

As illustrated in the examples below, the recombinant antibody fusion proteins of the disclosure retain the biological activities of the parent proteins, namely the ability to bind a human IgG conferred by the anti-human IgG antibody fragment and the catalytic activity of the invertase to convert sucrose to glucose. Thus, in some embodiments, the recombinant antibody fusion protein of the disclosure is capable of binding to a human IgG and has a catalytic activity of converting sucrose to glucose.

Also provided are nucleic acid molecules encoding any of the genetic fusions of an anti-human IgG antibody fragment and an invertase, or any of the polypeptides, disclosed herein. In some embodiments, the nucleic acid molecule comprises a nucleic acid sequence having at least about 70%, 75%, 80%, 85%, 90%, 95%, 96%, 97%, 98% or 99% sequence identity to SEQ ID NO: 25, SEQ ID NO: 27, SEQ ID NO: 29, or SEQ ID NO: 31. In some embodiments, the nucleic acid molecule comprises the nucleic acid sequence of SEQ ID NO: 25, SEQ ID NO: 27, SEQ ID NO: 29, or SEQ ID NO: 31. In some embodiments, the nucleic acid molecule of the disclosure may further comprise one or a plurality of regulatory sequences operably linked to the nucleic acid sequence encoding the genetic fusion. Examples of regulatory sequences include, but not limited to, promoters, enhancers and other expression control elements (e.g., polyadenylation signals). Further examples of regulatory sequences are described in, for example, Goeddel, 1990, Gene Expression Technology: Methods in Enzymology 185, Academic Press, San Diego, Calif. and Baron et al., 1995, Nucleic Acids Res. 23:3605-06.

In some embodiments, the nucleic acid molecule of the disclosure may be comprised in a vector. For example, the nucleic acid molecule of the disclosure can be part of a plasmid and thus the nucleic acid molecule is a plasmid comprising the nucleic acid sequence encoding the genetic fusion. Accordingly, provided herein is a vector or plasmid that is capable of expressing any of the genetic fusions of an anti-human IgG antibody fragment and an invertase, or any of the polypeptides, disclosed herein. The vector may be a plasmid. The plasmid may be useful for transfecting cells with nucleic acids encoding the genetic fusions of an anti-human IgG antibody fragment and an invertase, or any of the polypeptides, disclosed herein, which the transformed host cell is cultured and maintained under conditions to express the genetic fusion proteins.

Accordingly, the disclosure also relates to one or a plurality of cells that comprise any of the disclosed nucleic acid molecules, plasmids, or vectors. In some embodiments, the cells of the present disclosure are cultured cells comprising one or more of the disclosed nucleic acid molecules, plasmids, or vectors. In such embodiments, the cells may include, but not limited to, bacterial cells, fungal cells, insect cells, mammalian cells, or human cells. Mammalian cell lines available as hosts for expression of the disclosed nucleic acid molecules, plasmids, or vectors to produce any of the genetic fusions of an anti-human IgG antibody fragment and an invertase, or any of the recombinant antibody fusion protein, disclosed herein are well known in the art and include many immortalized cell lines available from the American Type Culture Collection (ATCC). These include, inter alia, Chinese hamster ovary (CHO) cells, NSO, SP2 cells, HeLa cells, baby hamster kidney (BHK) cells, monkey kidney cells (COS), human hepatocellular carcinoma cells (e.g., Hep G2), A549 cells, 3T3 cells, human embryo kidney 293 (HEK-293) cells and a number of other cell lines. Cell lines of particular preference are selected through determining which cell lines have high expression levels. In some embodiments, the host cell is a HEK-293 host cell. Other cell lines that may be used are insect cell lines, such as SD cells, amphibian cells, bacterial cells, plant cells, filamentous fungus cells (e.g., Trichoderma reesei), and yeast cells (e.g., Saccharomyces cerevisiae or Pichia pastoris). In some embodiments, the host cell may be a prokaryote host cell such as E. coli. To produce the recombinant antibody fusion protein of the disclosure, the cells may be co-transfected or co-transformed with a nucleic acid molecule, plasmid, or vector encoding a heavy chain and a second nucleic acid molecule, plasmid, or vector encoding a light chain, in which either the heavy chain or the light chain is a genetic fusion of an anti-human IgG antibody fragment and an invertase described herein. Any method routinely used by one of ordinary skill in the art for transforming or transfecting cells and maintaining transformed or transfected cells can be used to generate the cells of the disclosure.

The recombinant antibody fusion proteins of the disclosure produced in the host cells can be recovered from the culture medium using standard protein purification methods. Further, expression of the recombinant antibody fusion proteins of the disclosure from production cell lines can be enhanced using a number of known techniques. For example, the glutamine synthetase gene expression system (the GS system) is a common approach for enhancing expression under certain conditions.

### Kits

The recombinant antibody fusion proteins of the disclosure described above as well as other materials can be packaged together in any suitable combination as a kit useful for performing, or aiding in the performance of, the methods of use described elsewhere herein. It is useful if the kit components in a given kit are designed and adapted for use together in any of the disclosed methods. For example, disclosed are kits comprising one or more recombinant antibody fusion proteins of the disclosure and an antigenic polypeptide immobilized or absorbed onto a solid support. Any rigid or semi-rigid support onto which molecules (e.g., nucleic acids, polypeptides, mimetics) may be bound can be used as the solid support to immobilize or absorb the antigenic polypeptide comprised in the kits of the disclosure. Examples of solid supports include, but not limited to, membranes, filters, chips, slides, wafers, fibers, magnetic, or nonmagnetic beads, gels, capillaries or other tubing, plates, polymers, and microparticles with a variety of surface forms including wells, trenches, pins, channels, and pores. In some embodiments, the solid support is a strip. In some embodiments, the solid support is a bead. In some embodiments, the solid support is a plate. In some embodiments, the solid support is a multi-well plate. In some embodiments, the solid support is a slide.

The antigenic polypeptide immobilized or absorbed onto the solid support comprised in the kits of the disclosure may be any polypeptide that is capable of generating an immune response (e.g., the production of antibodies) in a subject. In some embodiments, the antigenic polypeptide is a pathogen-specific polypeptide. In some embodiments, the antigenic polypeptide is a polypeptide of the SARS-COV-2. In some embodiments, the antigenic polypeptide is the receptor binding domain (RBD) of the SARS-COV-2. In some embodiments, the antigenic polypeptide is a polypeptide of human origin, such as a polypeptide associated with autoimmune diseases or disorders (e.g., stress proteins).

In some embodiments, the kits of the disclosure may further comprise one or more buffer solutions, such as a solution comprising sucrose. In some embodiments, the kits of the disclosure may comprise a solution comprising sucrose. In some embodiments, the kits of the disclosure may further comprise a device for collecting a sample from a subject and/or a container for housing the sample. In some embodiments, the kits of the disclosure may further comprise a glucometer. Any glucometer available in the market may be used. In some embodiments, the kits of the disclosure may further comprise instructions for use.

In some embodiments, the kits of the disclosure is configured as a point-of-care diagnostic kit. In some embodiments, the kits of the disclosure is configured as an over-the-counter diagnostic kit.

### Methods of Use

Disclosed are methods of detecting an antibody to an antigenic polypeptide in a sample using any of the recombinant antibody fusion proteins disclosed herein. In some embodiments, the methods detect an antibody to an antigenic polypeptide of an infectious pathogen, such as a bacterium, virus, viroid, protozoan, prion, or fungus. In some embodiments, the infectious pathogen comprises a severe acute respiratory syndrome coronavirus-2 (SARS-CoV-2). In such embodiments, the methods detect an antibody to an antigenic polypeptide of SARS-COV-2, such as the receptor binding domain (RBD) of the SARS-COV-2 spike protein. In some embodiments, the methods detect an antibody to an antigenic polypeptide that is a polypeptide of human origin, such as a polypeptide associated with autoimmune diseases or disorders (e.g., stress proteins). In any of these methods, the methods comprise: a) contacting the sample with the antigenic polypeptide under conditions sufficient for the antibody to bind the antigenic polypeptide to produce a bound antibody; b) contacting the bound antibody with at least one recombinant antibody fusion protein of the disclosure to produce a binding complex, wherein the at least one recombinant antibody fusion protein is capable of binding to a human IgG and has an invertase catalytic activity of converting at least one substrate into at least one detectable product; c) contacting the binding complex with the at least one substrate under conditions sufficient to convert the at least one substrate into the at least one detectable product; and d) detecting the presence or absence of the at least one detectable product in the sample, wherein detecting the presence of the at least one detectable product in the sample indicates that the sample contains the antibody to the antigenic polypeptide. In some embodiments, the methods further comprise obtaining the sample from a subject. Any sample can be used for the detection. In some embodiments, the sample is an oral fluid, nasal fluid, saliva, blood, plasma, or serum. Determination of conditions sufficient for an antibody to bind an antigenic polypeptide to produce a bound antibody is within the skill and knowledge of one or ordinary skill in the art. Similarly, determination of conditions sufficient for an invertase or a binding complex comprising the same to convert the at least one substrate into the at least one detectable product is also within the skill and knowledge of one or ordinary skill in the art. In some embodiments, the at least one substrate comprises sucrose. In some embodiments, the at least one detectable product comprise glucose. In some embodiments, the presence of the at least one detectable product in the sample is detected using a glucometer. In some embodiments, the method is conducted as a point-of-care diagnostic test using a personal portable glucometer. Any glucometer available in the market may be used.

The disclosed methods of detecting an antibody to an antigenic polypeptide in a sample can be conducted using any of the kits disclosed herein. Accordingly, in some embodiments, provided herein is a method of detecting an antibody to an antigenic polypeptide in a sample, the method comprising detecting the presence or absence of the antibody in the sample using any of the kits disclosed herein. In some embodiments, the antigenic polypeptide is a polypeptide of an infectious pathogen. In some embodiments, the antigenic polypeptide is polypeptide of SARS-COV-2. In some embodiments, the antigenic polypeptide is a polypeptide of human origin. In some embodiments, the antigenic polypeptide is a polypeptide of human origin associated with an autoimmune disease or disorder.

Also disclosed are methods of diagnosing an infection of a pathogen in a subject in need thereof using any of the recombinant antibody fusion proteins disclosed herein. In some embodiments, the infection diagnosed by the disclosed methods is caused by an infectious pathogen, such as a bacterium, virus, viroid, protozoan, prion, or fungus. In some embodiments, the infectious pathogen comprises SARS-COV-2. In any of these methods, the methods comprise: a) contacting a sample of the subject with at least one polypeptide of the pathogen under conditions sufficient for an antibody to the at least one polypeptide of the pathogen to bind the at least one polypeptide of the pathogen to produce a bound antibody; b) contacting the bound antibody with at least one recombinant antibody fusion protein of the disclosure to produce a binding complex, wherein the at least one recombinant antibody fusion protein is capable of binding to a human IgG and has a catalytic activity of converting at least one substrate into at least one detectable product; c) contacting the binding complex with the at least one substrate under conditions sufficient to convert the at least one substrate into the at least one detectable product; and d) detecting the presence or absence of the at least one detectable product in the sample of the subject, wherein detecting the presence of the at least one detectable product in the sample indicates that the subject has an infection of the pathogen. In some embodiments, the methods further comprise obtaining the sample from the subject. Any sample can be used for the diagnosis. In some embodiments, the sample is an oral fluid, nasal fluid, saliva, blood, plasma, or serum. Determination of conditions sufficient for an antibody to bind at least one polypeptide of the pathogen to produce a bound antibody is within the skill and knowledge of one or ordinary skill in the art. Similarly, determination of conditions sufficient for an invertase or a binding complex comprising the same to convert the at least one substrate into the at least one detectable product is also within the skill and knowledge of one or ordinary skill in the art. In some embodiments, the at least one substrate comprises sucrose. In some embodiments, the at least one detectable product comprise glucose. In some embodiments, the presence of the at least one detectable product in the sample is detected using a glucometer. In some embodiments, the method is conducted as a point-of-care diagnostic test using a personal portable glucometer. Any glucometer available in the market may be used.

The disclosed methods of diagnosing a pathogenic infection in a subject in need thereof can be conducted using any of the kits disclosed herein. Accordingly, in some embodiments, provided herein is a method of diagnosing a pathogenic infection in a subject in need thereof, the method comprising detecting the presence or absence of an antibody to at least one pathogen-specific polypeptide in a sample of the subject using any of the kits disclosed herein. In some embodiments, the pathogenic infection is caused by SARS-COV-2. In some embodiments, the pathogenic infection is caused by SARS-COV-2 and the at least one pathogen-specific polypeptide immobilized or absorbed onto the solid support of the disclosed kits is the receptor binding domain (RBD) of the SARS-COV-2 spike protein.

Further disclosed are methods of treating a pathogenic infection in a subject in need thereof, the method comprising diagnosing the subject as having the pathogenic infection by any of the methods disclosed herein and administering to the subject a therapeutically effective amount of treatment for the pathogenic infection. Any treatment for combating the pathogenic infection may be used herein. In some embodiments, the treatment for the pathogenic infection comprises resting. In some embodiments, the treatment for the pathogenic infection comprises administering to the subject a therapeutically effective amount of one or more antibiotics, antivirals, or antifungals, depending on the pathogenic infection diagnosed.

The disclosed methods of treating a pathogenic infection in a subject in need thereof can be conducted using any of the kits disclosed herein. Accordingly, in some embodiments, provided herein is a method of treating a pathogenic infection in a subject in need thereof, the method comprising detecting the presence or absence of an antibody to at least one pathogen-specific polypeptide in a sample of the subject using any of the kits disclosed herein and administering to the subject a therapeutically effective amount of treatment for the pathogenic infection if the presence of the at least one pathogen-specific polypeptide is detected. In some embodiments, the treatment for the pathogenic infection comprises resting. In some embodiments, the treatment for the pathogenic infection comprises administering to the subject a therapeutically effective amount of one or more antibiotics, antivirals, or antifungals.

### EXAMPLES

### Example 1. Recombinant Antibodies Comprising Anti-Human IgG-Invertase Fusion Proteins for Quantitative Immunosensing Using Glucometers

Rapid diagnostics that can accurately inform patients of disease risk are critical to mitigating the spread of the current COVID-19 and future pandemics. To be effective, such diagnostics must rely on simple, cost-effective, and widely available electronics. Preferably, the platforms should be compatible with existing telehealth infrastructure, thus enabling both easy diagnosis access and remote care. To overcome the high cost, time, and trained personnel requirements of most benchtop-based antibody assays, glucose monitors have been singled out as ideal detectors. The idea is to use enzymatic reporters that, when bound to disease-specific patient antibodies, produce glucose in direct proportion to the number of antibodies present in the sample. Although a simple concept, the coupling of enzymatic reporters to secondary antibodies or antigens often results in low yields, unknown stoichiometry, and poor binding and catalytic efficiencies. Addressing this problem, disclosed herein is a first fusion protein consisting of anti-human immunoglobulin G (IgG) symmetrically linked to two invertases via short peptide linkers. The resulting fusion protein retains the binding affinity and catalytic activity of the parent proteins, and serves as an excellent reporter for immunoassays. Using this fusion protein, glucometer-based measurement of anti-SARS-COV-2 spike protein antibodies in blinded clinical training sets is demonstrated. The results demonstrate the ability of the reporter to report patient IgGs in serum with excellent agreement relative to commercial ELISAs. Because the disclosed fusion protein could act as a secondary antibody-reporter system in any immunoassay, it represents a general tool for the detection of antibodies against other diseases beyond SARS-COV-2.

### 1. Materials and Methods

i. Protein Expression and Purification

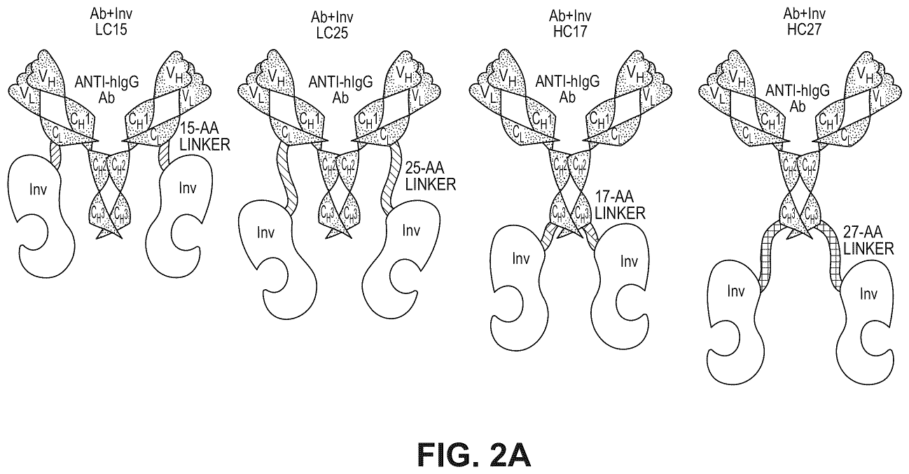

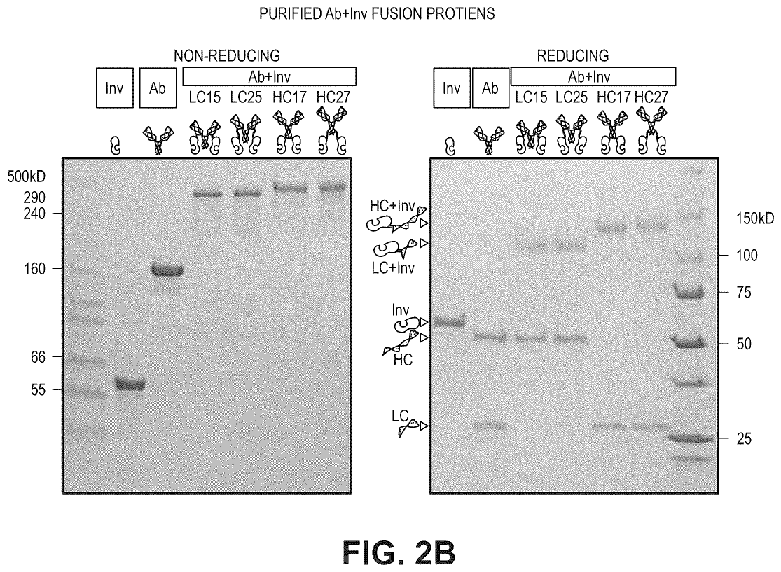

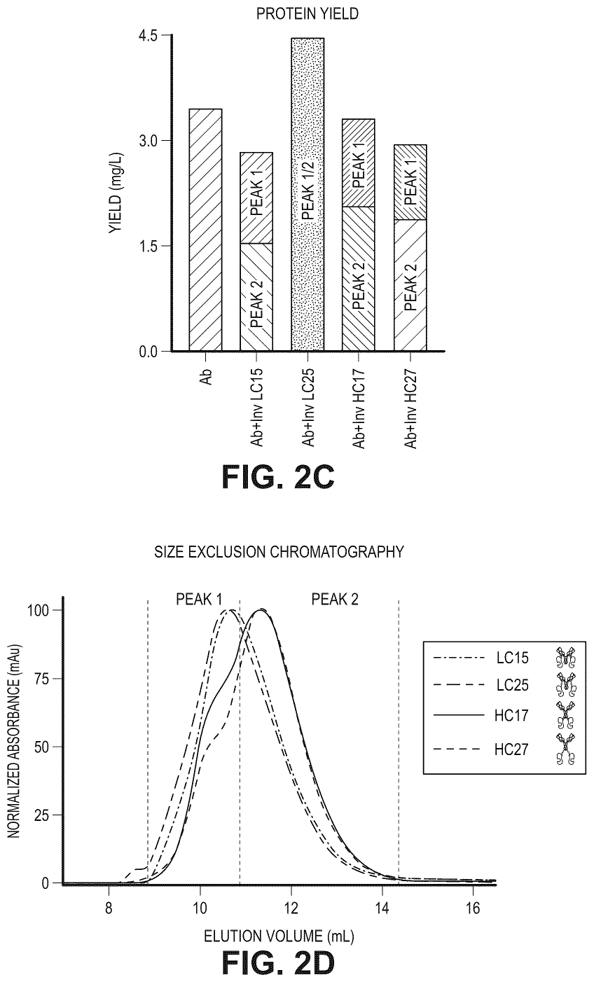

The HP6017 mouse IgG 2a kappa hybridoma (Jefferis et al., Immunol. Lett., 1985, 10 (3-4): 223-252 (doi: 10.1016/0165-2478 (85) 90082-3; PMID: 3899923) and Reimer et al., Hybridoma, 1984, 3 (3): 263-275 (doi: 10.1089/hyb.1984.3.263; PMID: 6209201) was purchased from ATCC and sequencing of the variable domains was performed by GenScript. Briefly, total RNA was isolated from the HP6017 hybridoma cells following the technical manual of RNeasy Plus Micro Kit (Qiagen). Total RNA was then reverse-transcribed into cDNA using either isotype-specific anti-sense primers or universal primers following the technical manual of SMARTScribe Reverse Transcriptase (TaKaRa). Antibody fragments of heavy chain and light chain were amplified according to the standard operating procedure (SOP) of rapid amplification of cDNA ends (RACE). Amplified antibody fragments were cloned into a standard cloning vector separately. Colony PCR was performed to screen for clones with inserts of correct sizes. The consensus sequence was provided. The recombinant antibody, HP6017, was formulated as mouse immunoglobulin (IgG) 2a kappa isotype to match the parent clone. The heavy chain (HC) and light chain (LC) were separately cloned into the gWiz vector (Genlantis). For enzyme fusion proteins, the full, intracellular isoform of Saccharomyces cerevisiae invertase (UniProt ID, P00724-2) was fused to C-terminus of the LC by a flexible (G4S) 3 or (G4S) 5 linker (denoted LC15 or LC25, respectively), or to the C-terminus of the HC by an AS (G4S) 3 or AS (G4S) 5 linker (denoted HC17 or HC27, respectively; FIG. 2A).

The sequences of constructs used in this Example are summarized the Table below.

Ab HC (clone HP6017): signal sequence-Ab V_{H}-mouse IgG2a C_{H}1, C_{H}2, and C_{H}3
(SEQ ID NO: 22)
MGWSCIILFLVATATGVHSQVQLQQPGAELVKPGASVKMSCKASGYTFTNYWINWV
KQRPGQGLEWIGDIYPGGGITNYNEKFKTKATLTLDTSSSTVYMQLSSLTSEDSA
VYYCSRSYGKYFDYWGQGTTLIVSSAKTTAPSVYPLAPVCGDTTGSSVTLGCLVKG
YFPEPVTLTWNSGSLSSGVHTFPAVLQSDLYTLSSSVTVTSSTWPSQSITCNVAHPASS
TKVDKKIEPRGPTIKPCPPCKCPAPNLLGGPSVFIFPPKIKDVLMISLSPIVTCVVVDVSE
DDPDVQISWFVNNVEVHTAQTQTHREDYNSTLRVVSALPIQHQDWMSGKEFKCKVN
NKDLPAPIERTISKPKGSVRAPQVYVLPPPEEEMTKKQVTLTCMVTDFMPEDIYVEWT
NNGKTELNYKNTEPVLDSDGSYFMYSKLRVEKKNWVERNSYSCSVVHEGLHNHHTT
KSFSRTPGK
(SEQ ID NO: 21)
Atgggatggagctgtatcatcctctttttggtagcaacagctacaggtgtccactcccaggtccaactgcagcagcctggggctgag
cttgtgaagcctggggcttcagtgaagatgtcctgcaaggctagcggctacaccttcaccaactactggataaactgggtgaag
cagaggcctggacaaggtctagagtggattggagatatttatcctggtggaggtattactaattacaatgagaagttcaagacc
aaggccacactgactctggacacatcctcgtcgacagtctatatgcaactcagcagcctgacatctgaagactctgcggtatact
attgttcaagatcctatggtaaatatttcgactactggggccaaggcaccactctcatagtctcctcagccaaaacaacggcgcca
tcggtctatccactggcccctgtgtgtggagatacaactggctcgtcggtgactctaggatgcctggtcaagggttatttccctgagccagt
gaccttgacctggaactctggttccctgtccagtggtgtgcacaccttcccagctgtcctccagtctgacctctacaccctcagcagctca
gtgactgtaacctcgagcacctggcccagccagtccatcacctgcaatgtggcccacccggcaagcagcaccaaggtggacaagaaa
attgagcccagagggcccacaatcaagccctgtcctccatgcaaatgcccagcacctaacctcttgggtggaccatccgtcttcatcttcc
ctccaaagatcaaggatgtactcatgatctccctgagccccatagtcacatgtgtggtggtggatgtgagcgaggatgacccagatgtcc
agatcagctggtttgtgaacaacgtggaagtacacacagctcagacacaaacccatagagaggattacaacagtactctccgggtggtc
agtgccctccccatccagcaccaggactggatgagtggcaaggagttcaaatgcaaggtcaacaacaaagacctcccagcgcccatc
gagagaaccatctcaaaacccaaagggtcagtaagagctccacaggtatatgtcttgcctccaccagaagaagagatgactaagaaac
aggtcactctgacctgcatggtcacagacttcatgcctgaagacatttacgtggagtggaccaacaacgggaaaacagagctaaactac
aagaacactgaaccagtcctggactctgatggttcttacttcatgtacagcaagctgagagtggaaaagaagaactgggtggaaagaaa
tagctactcctgttcagtggtccacgagggtctgcacaatcaccacacgactaagagcttctcccggactccgggtaaa
Ab LC (clone HP6017): signal sequence-Ab V_{L}-mouse Kappa C_{L}
(SEQ ID NO: 24)
MRAPAQFFGILLLWFPGIRCDIKMTQSPSSMYASVGERVTITCKASQDIKSYLTWYQ
KKPWKSPRTLIFYSTRLADGVPSRFSGSGSGQDFSLTISSLESDDTATYYCLQHDE
SPFTFGGGTKLEIKRADAAPTVSIFPPSSEQLTSGGASVVCFLNNFYPKDINVKWKID
GSERQNGVLNSWTDQDSKDSTYSMSSTLTLTKDEYERHNSYTCEATHKTSTSPIVKSF
NRNEC
(SEQ ID NO: 23)
Atgagggcccctgctcagttttttgggatcttgttgctctggtttccaggtatcagatgtgatatcaagatgacccagtccccatcctcc
atgtatgcatcggtgggagagagagtcactatcacgtgcaaggcgagtcaggacattaaaagctatttaacctggtaccagaa
gaagccatggaaatctcctaggaccctgatcttctattcaacaaggttggcagatggggtcccatcaagattcagtggcagtgg
atctgggcaagatttttctctaaccatcagcagcctggagtctgacgatacagcaacttattactgtctccagcatgatgagagcc
cgttcacgttcggaggagggaccaagctggaaataaaacgggctgatgctgcaccaactgtatccatcttcccaccatccagtgag
cagttaacatctggaggtgcctcagtcgtgtgcttcttgaacaacttctaccccaaagacatcaatgtcaagtggaagattgatggcagtg
aacgacaaaatggcgtcctgaacagttggactgatcaggacagcaaagacagcacctacagcatgagcagcaccctcacgttgacca
aggacgagtatgaacgacataacagctatacctgtgaggccactcacaagacatcaacttcacccattgtcaagagcttcaacaggaat
gagtgt
Ab + Inv HC17: signal sequence-Ab V_{H}-mouse IgG2a C_{H}1, C_{H}2, and C_{H}3-Linker-Invertase
(SEQ ID NO: 26)
MGWSCIILFLVATATGVHSQVQLQQPGAELVKPGASVKMSCKASGYTFTNYWINWV
KQRPGQGLEWIGDIYPGGGITNYNEKFKTKATLTLDTSSSTVYMQLSSLTSEDSA
VYYCSRSYGKYFDYWGQGTTLIVSSAKTTAPSVYPLAPVCGDTTGSSVTLGCLVKG
YFPEPVTLTWNSGSLSSGVHTFPAVLQSDLYTLSSSVTVTSSTWPSQSITCNVAHPASS
TKVDKKIEPRGPTIKPCPPCKCPAPNLLGGPSVFIFPPKIKDVLMISLSPIVTCVVVDVSE
DDPDVQISWFVNNVEVHTAQTQTHREDYNSTLRVVSALPIQHQDWMSGKEFKCKVN
NKDLPAPIERTISKPKGSVRAPQVYVLPPPEEEMTKKQVTLTCMVTDFMPEDIYVEWT
NNGKTELNYKNTEPVLDSDGSYFMYSKLRVEKKNWVERNSYSCSVVHEGLHNHHTT
KSFSRTPGKASGGGGSGGGGSGGGGSMTNETSDRPLVHFTPNKGWMNDPNGLWYDE
KDAKWHLYFQYNPNDTVWGTPLFWGHATSDDLTNWEDQPIAIAPKRNDSGAFSGSM
VVDYNNTSGFFNDTIDPRQRCVAIWTYNTPESEEQYISYSLDGGYTFTEYQKNPVLAA
NSTQFRDPKVFWYEPSQKWIMTAAKSQDYKIEIYSSDDLKSWKLESAFANEGFLGYQ
YECPGLIEVPTEQDPSKSYWVMFISINPGAPAGGSFNQYFVGSFNGTHFEAFDNQSRV
VDFGKDYYALQTFFNTDPTYGSALGIAWASNWEYSAFVPTNPWRSSMSLVRKFSLNT
EYQANPETELINLKAEPILNISNAGPWSRFATNTTLTKANSYNVDLSNSTGTLEFELVY
AVNTTQTISKSVFADLSLWFKGLEDPEEYLRMGFEVSASSFFLDRGNSKVKFVKENPY
FTNRMSVNNQPFKSENDLSYYKVYGLLDQNILELYFNDGDVVSTNTYFMTTGNALGS
VNMTTGVDNLFYIDKFQVREVK
(SEQ ID NO: 25)
atgggatggagctgtatcatcctctttttggtagcaacagctacaggtgtccactcccaggtccaactgcagcagcctggggctgag
cttgtgaagcctggggcttcagtgaagatgtcctgcaaggctagcggctacaccttcaccaactactggataaactgggtgaag
cagaggcctggacaaggtctagagtggattggagatatttatcctggtggaggtattactaattacaatgagaagttcaagacc
aaggccacactgactctggacacatcctcgtcgacagtctatatgcaactcagcagcctgacatctgaagactctgcggtatact
attgttcaagatcctatggtaaatatttcgactactggggccaaggcaccactctcatagtctcctcagccaaaacaacggcgcca
tcggtctatccactggcccctgtgtgtggagatacaactggctcgtcggtgactctaggatgcctggtcaagggttatttccctgagccagt
gaccttgacctggaactctggttccctgtccagtggtgtgcacaccttcccagctgtcctccagtctgacctctacaccctcagcagctca
gtgactgtaacctcgagcacctggcccagccagtccatcacctgcaatgtggcccacccggcaagcagcaccaaggtggacaagaaa
attgagcccagagggcccacaatcaagccctgtcctccatgcaaatgcccagcacctaacctcttgggtggaccatccgtcttcatcttcc
ctccaaagatcaaggatgtactcatgatctccctgagccccatagtcacatgtgtggtggtggatgtgagcgaggatgacccagatgtcc
agatcagctggtttgtgaacaacgtggaagtacacacagctcagacacaaacccatagagaggattacaacagtactctccgggtggtc
agtgccctccccatccagcaccaggactggatgagtggcaaggagttcaaatgcaaggtcaacaacaaagacctcccagcgcccatc
gagagaaccatctcaaaacccaaagggtcagtaagagctccacaggtatatgtcttgcctccaccagaagaagagatgactaagaaac
aggtcactctgacctgcatggtcacagacttcatgcctgaagacatttacgtggagtggaccaacaacgggaaaacagagctaaactac
aagaacactgaaccagtcctggactctgatggttcttacttcatgtacagcaagctgagagtggaaaagaagaactgggtggaaagaaa
tagctactcctgttcagtggtccacgagggtctgcacaatcaccacacgactaagagcttctcccggactccgggtaaagctagcggtg
gtggaggttcaggcgggggcggtagcggcggtgggggatccatgacaaacgaaactagcgatagacctttggtccacttcacaccc
aacaagggctggatgaatgacccaaatgggttgtggtacgatgaaaaagatgccaaatggcatctgtactttcaatacaacccaaatgac
accgtatggggtacgccattgttttggggccatgctacttccgatgatttgactaattgggaagatcaacccattgctatcgctcccaagcg
taacgattcaggtgctttctctggctccatggtggttgattacaacaacacgagtgggtttttcaatgatactattgatccaagacaaagatg
cgttgcgatttggacttataacactcctgaaagtgaagagcaatacattagctattctcttgatggtggttacacttttactgaataccaaaag
aaccctgttttagctgccaactccactcaattcagagatccaaaggtgttctggtatgaaccttctcaaaaatggattatgacggctgccaa
atcacaagactacaaaattgaaatttactcctctgatgacttgaagtcctggaagctagaatctgcatttgccaatgaaggtttcttaggctac
caatacgaatgtccaggtttgattgaagtcccaactgagcaagatccttccaaatcttattgggtcatgtttatttctatcaacccaggtgcac
ctgctggcggttccttcaaccaatattttgttgggtccttcaatggtactcattttgaagcgtttgacaatcaatctagagtggtagattttggta
aggactactatgccttgcaaactttcttcaacactgacccaacatacggttcagcattaggtattgcctgggcttcaaactgggagtacagt
gcctttgtcccaactaacccatggagatcatccatgtctttggtccgcaagttttctttgaacactgaatatcaagctaatccagagactgaat
tgatcaatttgaaagccgaaccaatattgaacattagtaatgctggtccctggtctcgttttgctactaacacaactctaactaaggccaattc
ttacaatgtcgatttgagcaactcgactggtaccctagagtttgagttggtttacgctgttaacaccacacaaaccatatccaaatccgtcttt
gccgacttatcactttggttcaagggtttagaagatcctgaagaatatttgagaatgggttttgaagtcagtgcttcttccttctttttggaccgt
ggtaactctaaggtcaagtttgtcaaggagaacccatatttcacaaacagaatgtctgtcaacaaccaaccattcaagtctgagaacgacc
taagttactataaagtgtacggcctactggatcaaaacatcttggaattgtacttcaacgatggagatgtggtttctacaaatacctacttcat
gaccaccggtaacgctctaggatctgtgaacatgaccactggtgtcgataatttgttctacattgacaagttccaagtaagggaagtaaaa
Ab + Inv HC27: signal sequence-Ab V_{H}-mouse IgG2a C_{H}1, C_{H}2, and C_{H}3-Linker-Invertase
(SEQ ID NO: 28)
MGWSCIILFLVATATGVHSQVQLQQPGAELVKPGASVKMSCKASGYTFTNYWINWV
KQRPGQGLEWIGDIYPGGGITNYNEKFKTKATLTLDTSSSTVYMQLSSLTSEDSA
VYYCSRSYGKYFDYWGQGTTLIVSSAKTTAPSVYPLAPVCGDTTGSSVTLGCLVKG
YFPEPVTLTWNSGSLSSGVHTFPAVLQSDLYTLSSSVTVTSSTWPSQSITCNVAHPASS
TKVDKKIEPRGPTIKPCPPCKCPAPNLLGGPSVFIFPPKIKDVLMISLSPIVTCVVVDVSE
DDPDVQISWFVNNVEVHTAQTQTHREDYNSTLRVVSALPIQHQDWMSGKEFKCKVN
NKDLPAPIERTISKPKGSVRAPQVYVLPPPEEEMTKKQVTLTCMVTDFMPEDIYVEWT
NNGKTELNYKNTEPVLDSDGSYFMYSKLRVEKKNWVERNSYSCSVVHEGLHNHHTT
KSFSRTPGKASGGGGSGGGGSGGGGSGGGGSGGGGSMTNETSDRPLVHFTPNKGWM
NDPNGLWYDEKDAKWHLYFQYNPNDTVWGTPLFWGHATSDDLTNWEDQPIAIAPK
RNDSGAFSGSMVVDYNNTSGFFNDTIDPRQRCVAIWTYNTPESEEQYISYSLDGGYTF
TEYQKNPVLAANSTQFRDPKVFWYEPSQKWIMTAAKSQDYKIEIYSSDDLKSWKLES
AFANEGFLGYQYECPGLIEVPTEQDPSKSYWVMFISINPGAPAGGSFNQYFVGSFNGT
HFEAFDNQSRVVDFGKDYYALQTFFNTDPTYGSALGIAWASNWEYSAFVPTNPWRSS
MSLVRKFSLNTEYQANPETELINLKAEPILNISNAGPWSRFATNTTLTKANSYNVDLSN
STGTLEFELVYAVNTTQTISKSVFADLSLWFKGLEDPEEYLRMGFEVSASSFFLDRGNS
KVKFVKENPYFTNRMSVNNQPFKSENDLSYYKVYGLLDQNILELYFNDGDVVSTNTY
FMTTGNALGSVNMTTGVDNLFYIDKFQVREVK
(SEQ ID NO: 27)
atgggatggagctgtatcatcctctttttggtagcaacagctacaggtgtccactcccaggtccaactgcagcagcctggggctgag
cttgtgaagcctggggcttcagtgaagatgtcctgcaaggctagcggctacaccttcaccaactactggataaactgggtgaag
cagaggcctggacaaggtctagagtggattggagatatttatcctggtggaggtattactaattacaatgagaagttcaagacc
aaggccacactgactctggacacatcctcgtcgacagtctatatgcaactcagcagcctgacatctgaagactctgcggtatact
attgttcaagatcctatggtaaatatttcgactactggggccaaggcaccactctcatagtctcctcagccaaaacaacggcgcca
tcggtctatccactggcccctgtgtgtggagatacaactggctcgtcggtgactctaggatgcctggtcaagggttatttccctgagccagt
gaccttgacctggaactctggttccctgtccagtggtgtgcacaccttcccagctgtcctccagtctgacctctacaccctcagcagctca
gtgactgtaacctcgagcacctggcccagccagtccatcacctgcaatgtggcccacccggcaagcagcaccaaggtggacaagaaa
attgagcccagagggcccacaatcaagccctgtcctccatgcaaatgcccagcacctaacctcttgggtggaccatccgtcttcatcttcc
ctccaaagatcaaggatgtactcatgatctccctgagccccatagtcacatgtgtggtggtggatgtgagcgaggatgacccagatgtcc
agatcagctggtttgtgaacaacgtggaagtacacacagctcagacacaaacccatagagaggattacaacagtactctccgggtggtc
agtgccctccccatccagcaccaggactggatgagtggcaaggagttcaaatgcaaggtcaacaacaaagacctcccagcgcccatc
gagagaaccatctcaaaacccaaagggtcagtaagagctccacaggtatatgtcttgcctccaccagaagaagagatgactaagaaac
aggtcactctgacctgcatggtcacagacttcatgcctgaagacatttacgtggagtggaccaacaacgggaaaacagagctaaactac
aagaacactgaaccagtcctggactctgatggttcttacttcatgtacagcaagctgagagtggaaaagaagaactgggtggaaagaaa
tagctactcctgttcagtggtccacgagggtctgcacaatcaccacacgactaagagcttctcccggactccgggtaaagctagcggtg
gtggaggttcagggggtggaggatctggcggagggggttcaggcgggggcggtagcggcggtgggggatccatgacaaacgaa
actagcgatagacctttggtccacttcacacccaacaagggctggatgaatgacccaaatgggttgtggtacgatgaaaaagatgccaa
atggcatctgtactttcaatacaacccaaatgacaccgtatggggtacgccattgttttggggccatgctacttccgatgatttgactaattg
ggaagatcaacccattgctatcgctcccaagcgtaacgattcaggtgctttctctggctccatggtggttgattacaacaacacgagtggg
tttttcaatgatactattgatccaagacaaagatgcgttgcgatttggacttataacactcctgaaagtgaagagcaatacattagctattctct
tgatggtggttacacttttactgaataccaaaagaaccctgttttagctgccaactccactcaattcagagatccaaaggtgttctggtatga
accttctcaaaaatggattatgacggctgccaaatcacaagactacaaaattgaaatttactcctctgatgacttgaagtcctggaagctag
aatctgcatttgccaatgaaggtttcttaggctaccaatacgaatgtccaggtttgattgaagtcccaactgagcaagatccttccaaatctta
ttgggtcatgtttatttctatcaacccaggtgcacctgctggcggttccttcaaccaatattttgttgggtccttcaatggtactcattttgaagc
gtttgacaatcaatctagagtggtagattttggtaaggactactatgccttgcaaactttcttcaacactgacccaacctacggttcagcatta
ggtattgcctgggcttcaaactgggagtacagtgcctttgtcccaactaacccatggagatcatccatgtctttggtccgcaagttttctttga
acactgaatatcaagctaatccagagactgaattgatcaatttgaaagccgaaccaatattgaacattagtaatgctggtccctggtctcgtt
ttgctactaacacaactctaactaaggccaattcttacaatgtcgatttgagcaactcgactggtaccctagagtttgagttggtttacgctgtt
aacaccacacaaaccatatccaaatccgtctttgccgacttatcactttggttcaagggtttagaagatcctgaagaatatttgagaatgggt
tttgaagtcagtgcttcttccttctttttggaccgtggtaactctaaggtcaagtttgtcaaggagaacccatatttcacaaacagaatgtctgt
caacaaccaaccattcaagtctgagaacgacctaagttactataaagtgtacggcctactggatcaaaacatcttggaattgtacttcaacg
atggagatgtggtttctacaaatacctacttcatgaccaccggtaacgctctaggatctgtgaacatgaccactggtgtcgataatttgttcta
cattgacaagttccaagtaagggaagtaaaa
Ab + Inv LC15: signal sequence-Ab V_{L}-mouse Kappa C_{L}-Linker-Invertase
(SEQ ID NO: 30)
MRAPAQFFGILLLWFPGIRCDIKMTQSPSSMYASVGERVTITCKASQDIKSYLTWYQ
KKPWKSPRTLIFYSTRLADGVPSRFSGSGSGQDFSLTISSLESDDTATYYCLQHDE
SPFTFGGGTKLEIKRADAAPTVSIFPPSSEQLTSGGASVVCFLNNFYPKDINVKWKID
GSERQNGVLNSWTDQDSKDSTYSMSSTLTLTKDEYERHNSYTCEATHKTSTSPIVKSF
NRNECGGGGSGGGGSGGGGSMTNETSDRPLVHFTPNKGWMNDPNGLWYDEKDAK
WHLYFQYNPNDTVWGTPLFWGHATSDDLTNWEDQPIAIAPKRNDSGAFSGSMVVDY
NNTSGFFNDTIDPRQRCVAIWTYNTPESEEQYISYSLDGGYTFTEYQKNPVLAANSTQF
RDPKVFWYEPSQKWIMTAAKSQDYKIEIYSSDDLKSWKLESAFANEGFLGYQYECPG
LIEVPTEQDPSKSYWVMFISINPGAPAGGSFNQYFVGSFNGTHFEAFDNQSRVVDFGK
DYYALQTFFNTDPTYGSALGIAWASNWEYSAFVPTNPWRSSMSLVRKFSLNTEYQAN
PETELINLKAEPILNISNAGPWSRFATNTTLTKANSYNVDLSNSTGTLEFELVYAVNTT
QTISKSVFADLSLWFKGLEDPEEYLRMGFEVSASSFFLDRGNSKVKFVKENPYFTNRM
SVNNQPFKSENDLSYYKVYGLLDQNILELYFNDGDVVSTNTYFMTTGNALGSVNMTT
GVDNLFYIDKFQVREVK
(SEQ ID NO: 29)
atgagggcccctgctcagttttttgggatcttgttgctctggtttccaggtatcagatgtgatatcaagatgacccagtccccatcctcca
tgtatgcatcggtgggagagagagtcactatcacgtgcaaggcgagtcaggacattaaaagctatttaacctggtaccagaag
aagccatggaaatctcctaggaccctgatcttctattcaacaaggttggcagatggggtcccatcaagattcagtggcagtggat
ctgggcaagatttttctctaaccatcagcagcctggagtctgacgatacagcaacttattactgtctccagcatgatgagagcccg
ttcacgttcggaggagggaccaagctggaaataaaacgggctgatgctgcaccaactgtatccatcttcccaccatccagtgagca
gttaacatctggaggtgcctcagtcgtgtgcttcttgaacaacttctaccccaaagacatcaatgtcaagtggaagattgatggcagtgaa
cgacaaaatggcgtcctgaacagttggactgatcaggacagcaaagacagcacctacagcatgagcagcaccctcacgttgaccaag
gacgagtatgaacgacataacagctatacctgtgaggccactcacaagacatcaacttcacccattgtcaagagcttcaacaggaatga
gtgtggtggtggaggttcagggggtggaggtagcggcggagggggatccatgacaaacgaaactagcgatagacctttggtccactt
cacacccaacaagggctggatgaatgacccaaatgggttgtggtacgatgaaaaagatgccaaatggcatctgtactttcaatacaacc
caaatgacaccgtatggggtacgccattgttttggggccatgctacttccgatgatttgactaattgggaagatcaacccattgctatcgctc
ccaagcgtaacgattcaggtgctttctctggctccatggtggttgattacaacaacacgagtgggtttttcaatgatactattgatccaagac
aaagatgcgttgcgatttggacttataacactcctgaaagtgaagagcaatacattagctattctcttgatggtggttacacttttactgaata
ccaaaagaaccctgttttagctgccaactccactcaattcagagatccaaaggtgttctggtatgaaccttctcaaaaatggattatgacgg
ctgccaaatcacaagactacaaaattgaaatttactcctctgatgacttgaagtcctggaagctagaatctgcatttgccaatgaaggtttctt
aggctaccaatacgaatgtccaggtttgattgaagtcccaactgagcaagatccttccaaatcttattgggtcatgtttatttctatcaaccca
ggtgcacctgctggcggttccttcaaccaatattttgttgggtccttcaatggtactcattttgaagcgtttgacaatcaatctagagtggtag
attttggtaaggactactatgccttgcaaactttcttcaacactgacccaacctacggttcagcattaggtattgcctgggcttcaaactggg
agtacagtgcctttgtcccaactaacccatggagatcatccatgtctttggtccgcaagttttctttgaacactgaatatcaagctaatccaga
gactgaattgatcaatttgaaagccgaaccaatattgaacattagtaatgctggtccctggtctcgttttgctactaacacaactctaactaag
gccaattcttacaatgtcgatttgagcaactcgactggtaccctagagtttgagttggtttacgctgttaacaccacacaaaccatatccaaa
tccgtctttgccgacttatcactttggttcaagggtttagaagatcctgaagaatatttgagaatgggttttgaagtcagtgcttcttccttcttttt
ggaccgtggtaactctaaggtcaagtttgtcaaggagaacccatatttcacaaacagaatgtctgtcaacaaccaaccattcaagtctgag
aacgacctaagttactataaagtgtacggcctactggatcaaaacatcttggaattgtacttcaacgatggagatgtggtttctacaaatacc
tacttcatgaccaccggtaacgctctaggatctgtgaacatgaccactggtgtcgataatttgttctacattgacaagttccaagtaaggga
agtaaaa
Ab + Inv LC25: signal sequence-Ab V_{L}-mouse Kappa C_{L}-Linker-Invertase
(SEQ ID NO: 32)
MRAPAQFFGILLLWFPGIRCDIKMTQSPSSMYASVGERVTITCKASQDIKSYLTWYQ
KKPWKSPRTLIFYSTRLADGVPSRFSGSGSGQDFSLTISSLESDDTATYYCLQHDE
SPFTFGGGTKLEIKRADAAPTVSIFPPSSEQLTSGGASVVCFLNNFYPKDINVKWKID
GSERQNGVLNSWTDQDSKDSTYSMSSTLTLTKDEYERHNSYTCEATHKTSTSPIVKSF
NRNECGGGGSGGGGSGGGGSGGGGSGGGGSMTNETSDRPLVHFTPNKGWMNDPNG
LWYDEKDAKWHLYFQYNPNDTVWGTPLFWGHATSDDLTNWEDQPIAIAPKRNDSG
AFSGSMVVDYNNTSGFFNDTIDPRQRCVAIWTYNTPESEEQYISYSLDGGYTFTEYQK
NPVLAANSTQFRDPKVFWYEPSQKWIMTAAKSQDYKIEIYSSDDLKSWKLESAFANE
GFLGYQYECPGLIEVPTEQDPSKSYWVMFISINPGAPAGGSFNQYFVGSFNGTHFEAF
DNQSRVVDFGKDYYALQTFFNTDPTYGSALGIAWASNWEYSAFVPTNPWRSSMSLV
RKFSLNTEYQANPETELINLKAEPILNISNAGPWSRFATNTTLTKANSYNVDLSNSTGT
LEFELVYAVNTTQTISKSVFADLSLWFKGLEDPEEYLRMGFEVSASSFFLDRGNSKVK
FVKENPYFTNRMSVNNQPFKSENDLSYYKVYGLLDQNILELYFNDGDVVSTNTYFMT
TGNALGSVNMTTGVDNLFYIDKFQVREVK
(SEQ ID NO: 31)
atgagggcccctgctcagttttttgggatcttgttgctctggtttccaggtatcagatgtgatatcaagatgacccagtccccatcctcca
tgtatgcatcggtgggagagagagtcactatcacgtgcaaggcgagtcaggacattaaaagctatttaacctggtaccagaag
aagccatggaaatctcctaggaccctgatcttctattcaacaaggttggcagatggggtcccatcaagattcagtggcagtggat
ctgggcaagatttttctctaaccatcagcagcctggagtctgacgatacagcaacttattactgtctccagcatgatgagagcccg
ttcacgttcggaggagggaccaagctggaaataaaacgggctgatgctgcaccaactgtatccatcttcccaccatccagtgagca
gttaacatctggaggtgcctcagtcgtgtgcttcttgaacaacttctaccccaaagacatcaatgtcaagtggaagattgatggcagtgaa
cgacaaaatggcgtcctgaacagttggactgatcaggacagcaaagacagcacctacagcatgagcagcaccctcacgttgaccaag
gacgagtatgaacgacataacagctatacctgtgaggccactcacaagacatcaacttcacccattgtcaagagcttcaacaggaatga
gtgtggtggtggaggttcagggggtggaggatctggcggagggggttcaggcgggggcggtagcggcggtgggggatccatgac
aaacgaaactagcgatagacctttggtccacttcacacccaacaagggctggatgaatgacccaaatgggttgtggtacgatgaaaaag
atgccaaatggcatctgtactttcaatacaacccaaatgacaccgtatggggtacgccattgttttggggccatgctacttccgatgatttga
ctaattgggaagatcaacccattgctatcgctcccaagcgtaacgattcaggtgctttctctggctccatggtggttgattacaacaacacg
agtgggtttttcaatgatactattgatccaagacaaagatgcgttgcgatttggacttataacactcctgaaagtgaagagcaatacattagc
tattctcttgatggtggttacacttttactgaataccaaaagaaccctgttttagctgccaactccactcaattcagagatccaaaggtgttctg
gtatgaaccttctcaaaaatggattatgacggctgccaaatcacaagactacaaaattgaaatttactcctctgatgacttgaagtcctggaa
gctagaatctgcatttgccaatgaaggtttcttaggctaccaatacgaatgtccaggtttgattgaagtcccaactgagcaagatccttccaa
atcttattgggtcatgtttatttctatcaacccaggtgcacctgctggcggttccttcaaccaatattttgttgggtccttcaatggtactcatttt
gaagcgtttgacaatcaatctagagtggtagattttggtaaggactactatgccttgcaaactttcttcaacactgacccaacctacggttca
gcattaggtattgcctgggcttcaaactgggagtacagtgcctttgtcccaactaacccatggagatcatccatgtctttggtccgcaagttt
tctttgaacactgaatatcaagctaatccagagactgaattgatcaatttgaaagccgaaccaatattgaacattagtaatgctggtccctgg
tctcgttttgctactaacacaactctaactaaggccaattcttacaatgtcgatttgagcaactcgactggtaccctagagtttgagttggttta
cgctgttaacaccacacaaaccatatccaaatccgtctttgccgacttatcactttggttcaagggtttagaagatcctgaagaatatttgag
aatgggttttgaagtcagtgcttcttccttctttttggaccgtggtaactctaaggtcaagtttgtcaaggagaacccatatttcacaaacaga
atgtctgtcaacaaccaaccattcaagtctgagaacgacctaagttactataaagtgtacggcctactggatcaaaacatcttggaattgta
cttcaacgatggagatgtggtttctacaaatacctacttcatgaccaccggtaacgctctaggatctgtgaacatgaccactggtgtcgata
atttgttctacattgacaagttccaagtaagggaagtaaaa
Inv: signal sequence-Invertase-Linker-6xHis
(SEQ ID NO: 34)
MRVPAQLLGLLLLWLPGARCMTNETSDRPLVHFTPNKGWMNDPNGLWYDEKDAKWH
LYFQYNPNDTVWGTPLFWGHATSDDLTNWEDQPIAIAPKRNDSGAFSGSMVVDYNN
TSGFFNDTIDPRQRCVAIWTYNTPESEEQYISYSLDGGYTFTEYQKNPVLAANSTQFRD
PKVFWYEPSQKWIMTAAKSQDYKIEIYSSDDLKSWKLESAFANEGFLGYQYECPGLIE
VPTEQDPSKSYWVMFISINPGAPAGGSFNQYFVGSFNGTHFEAFDNQSRVVDFGKDY
YALQTFFNTDPTYGSALGIAWASNWEYSAFVPTNPWRSSMSLVRKFSLNTEYQANPE
TELINLKAEPILNISNAGPWSRFATNTTLTKANSYNVDLSNSTGTLEFELVYAVNTTQT
ISKSVFADLSLWFKGLEDPEEYLRMGFEVSASSFFLDRGNSKVKFVKENPYFTNRMSV
NNQPFKSENDLSYYKVYGLLDQNILELYFNDGDVVSTNTYFMTTGNALGSVNMTTG
VDNLFYIDKFQVREVKAAAHHHHHH
(SEQ ID NO: 33)
atgagggtccccgctcagctcctggggctcctgctgctctggctcccaggtgcacgatgtatgacaaacgaaactagcgatagaccttt
ggtccacttcacacccaacaagggctggatgaatgacccaaatgggttgtggtacgatgaaaaagatgccaaatggcatctgtactttca
atacaacccaaatgacaccgtatggggtacgccattgttttggggccatgctacttccgatgatttgactaattgggaagatcaacccattg
ctatcgctcccaagcgtaacgattcaggtgctttctctggctccatggtggttgattacaacaacacgagtgggtttttcaatgatactattga
tccaagacaaagatgcgttgcgatttggacttataacactcctgaaagtgaagagcaatacattagctattctcttgatggtggttacactttt
actgaataccaaaagaaccctgttttagctgccaactccactcaattcagagatccaaaggtgttctggtatgaaccttctcaaaaatggatt
atgacggctgccaaatcacaagactacaaaattgaaatttactcctctgatgacttgaagtcctggaagctagaatctgcatttgccaatga
aggtttcttaggctaccaatacgaatgtccaggtttgattgaagtcccaactgagcaagatccttccaaatcttattgggtcatgtttatttctat
caacccaggtgcacctgctggcggttccttcaaccaatattttgttgggtccttcaatggtactcattttgaagcgtttgacaatcaatctaga
gtggtagattttggtaaggactactatgccttgcaaactttcttcaacactgacccaacctacggttcagcattaggtattgcctgggcttcaa
actgggagtacagtgcctttgtcccaactaacccatggagatcatccatgtctttggtccgcaagttttctttgaacactgaatatcaagcta
atccagagactgaattgatcaatttgaaagccgaaccaatattgaacattagtaatgctggtccctggtctcgttttgctactaacacaactct
aactaaggccaattcttacaatgtcgatttgagcaactcgactggtaccctagagtttgagttggtttacgctgttaacaccacacaaaccat
atccaaatccgtctttgccgacttatcactttggttcaagggtttagaagatcctgaagaatatttgagaatgggttttgaagtcagtgcttctt
ccttctttttggaccgtggtaactctaaggtcaagtttgtcaaggagaacccatatttcacaaacagaatgtctgtcaacaaccaaccattca
agtctgagaacgacctaagttactataaagtgtacggcctactggatcaaaacatcttggaattgtacttcaacgatggagatgtggtttcta
caaatacctacttcatgaccaccggtaacgctctaggatctgtgaacatgaccactggtgtcgataatttgttctacattgacaagttccaag
taagggaagtaaaagcggccgcgcaccatcaccaccatcac

Antibodies and antibody-enzyme fusion proteins were expressed recombinantly in human embryonic kidney (HEK) 293F cells via transient co-transfection of plasmids encoding the HC and LC or one of the LC-invertase fusion proteins, or the LC and one of the HC-invertase fusion proteins. Fused or unfused HC and LC plasmids were titrated in small-scale co-transfection tests to determine optimal DNA content ratios for large-scale expression. HEK 293F cells were grown to 1.2×10⁶ cells/mL and diluted to 1.0×10⁶ cells/mL on the day of transfection. Plasmid DNA and modified Poly(beta-amino ester) (PBAE) (43) were independently diluted to 0.05 and 3 mg/mL, respectively, in 25 mM MgAc₂ (pH 5), mixed and incubated at room temperature for 15 minutes. Subsequently, the DNA/PBAE mixture (42 mL per liter of cells) was added to a flask containing the diluted cells, which was then incubated at 37° C. with shaking for 5 days. Secreted protein was harvested from HEK 293F cell supernatants by Protein G affinity chromatography, followed by size-exclusion chromatography on an AKTA™ fast protein liquid chromatography (FPLC) instrument using a Superdex 200 column (Cytiva).

The receptor binding domain (RBD) of the WT SARS-COV-2 spike protein from the earliest lineage A virus (WT, YP_009724390.1, residues 319-541; NC_045512.2, A lincage), with its native signal sequence and a C-terminal hexahistidine tag in the pCAGGS plasmid (8) was used to produce RBD via transient transfection of HEK 293F cells, as described for the antibody and antibody-enzyme fusion proteins. Protein was purified via Ni-NTA (Expedcon) affinity chromatography followed by size-exclusion chromatography on a Superdex 200 column (Cytiva) using an FPLC instrument. All proteins were stored in HEPES-buffered saline (HBS, 150 mM NaCl in 10 mM HEPES pH 7.3). Purity was verified by SDS-PAGE analysis.

ii. Bio-Layer Interferometry Binding Studies

Biotinylated IgG from human scrum (Rockland Immunochemicals) was immobilized to streptavidin-coated tips for analysis on an Octet Red96 bio-layer interferometry (BLI) instrument (Sartorius). Less than 5 signal units (nm) of hIgG was immobilized to minimize mass transfer effects. PBSA (PBS pH 7.2 containing 0.1% BSA) was used for all dilutions and as dissociation buffer. Tips were exposed to serial dilutions of antibody, the various Ab+Inv fusion proteins, or S. cerevisiae invertase (as a negative control, NZYTech) in a 96-well plate for 300 seconds. Dissociation was then measured for 450 seconds. Surface regeneration for all interactions was conducted using 15 seconds exposure to 0.1 M glycine pH 3.0. Analysis and kinetic curve fitting (assuming a 1:1 binding model) were conducted using Octet Data Analysis HT software version 7.1 (Sartorius). Normalized equilibrium binding curves were obtained by plotting the response value at the end of the association phase for each sample dilution, dividing by the molecular weight of each ligand, and normalizing to the maximum value. Equilibrium binding curves were fitted and equilibrium Kp values determined using GraphPad Prism data analysis software v9.0, assuming all binding interactions to be first order. Experiments were performed at least twice with similar results.

iii. Enzyme Activity Testing

The enzymatic activity of the various Ab+Inv fusion proteins was compared to unfused invertase (Inv, NZYTech) by first incubating the various proteins with sucrose. The resulting concentration of glucose generated was measured using a commercially available StatStrip medical-grade glucometer (Nova Biomedical). All incubations and measurements were performed at room temperature with 30 μL reaction volumes, and unfused HP6017 (anti-hIgG) antibody was used as a negative control. A range of sucrose concentrations, incubation times, and enzyme concentrations were independently tested. The ranges spanned the values that yielded values within the glucometer detection limits. A 15-minute reaction time was used for the sucrose titration, with 0.166 μM of the Ab+Inv fusion protein, or 0.33 μM of Inv (for molar equivalence of the enzyme). For varying reaction times, 0.166 μM of the Ab+Inv fusion proteins or 0.33 μM of Inv were mixed with 250 mM sucrose. The enzyme concentration titration used a 15-minute reaction time, and 250 mM sucrose. Enzyme activity in the presence of 4-fold excess (0.66 μM) of hIgG was also measured, using a 15-minute reaction time, 250 mM sucrose, and 0.166 UM of the Ab+Inv fusion proteins or 0.33 μM of Inv. Data was plotted and fit using GraphPad Prism data analysis software v9.0. Experiments were performed at least three times with similar results.

iv. Fabrication of Immunoassay Strips

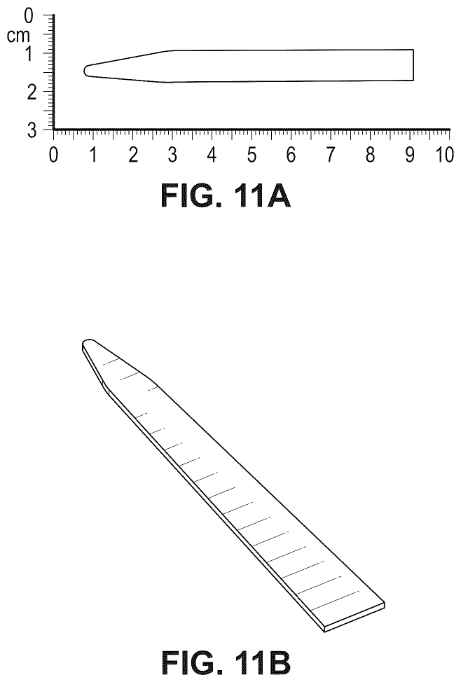

Acrylic sheets ( 1/16″ thick, part #8560K171, McMaster-Carr) were laser cut by Precision Microfab (Maryland, USA) to achieve a snug fit inside 1.5 mL microcentrifuge tubes (ScalRite) (FIG. 11A). Prior to functionalization, each strip was cleaned using 100% ethanol to remove any plastic particles remaining from the cutting process. Production of an antifouling hydrogel on the surface of the strips was adapted from a previously reported protocol (28). Briefly, each strip was immersed inside a 1.5 mL microcentrifuge tube containing 50 μL of a solution consisting of 5 mg/mL bovine serum albumin (BSA) and 1% (v/v) glutaraldehyde (GA) (Sigma) in phosphate-buffered saline (1× PBS, 11.9 mM phosphate, 137 mM NaCl, 2.7 mM KCl, pH 7.4). The strips were incubated in this solution overnight (18 hours) at 4° C. The next day, the strips were rinsed using a jet of deionized water from a squirt bottle for 5 seconds on each side.

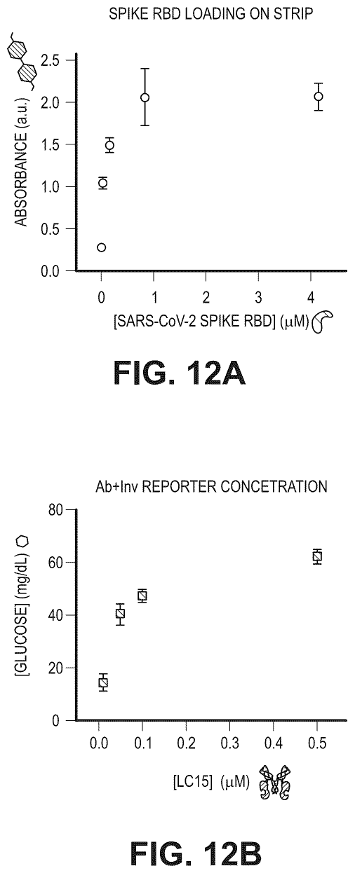

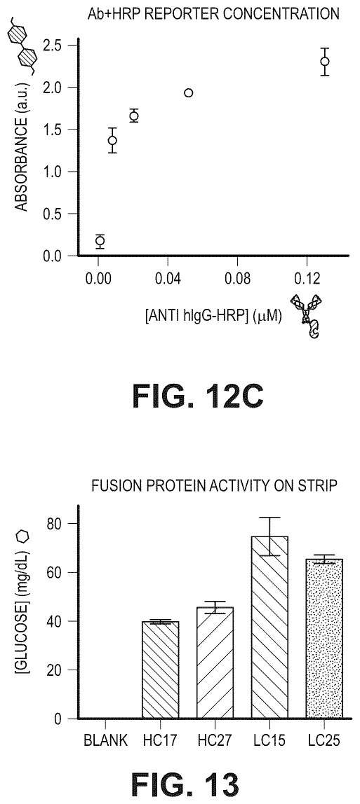

To functionalize the antifouling hydrogel with antigen, the strips were immersed in tubes containing 50 μL of a solution consisting of 400 mM 1-Ethyl-3-(3-dimethylaminopropyl) carbodiimide (EDC) and 200 mM N-hydroxysuccinimide (NHS) (Sigma) in 1×PBS. Tubes were stirred at 1,500 rpm and 25° C. for 30 minutes using a Thermomixer F1.5 (Fisher Scientific). Without further washing, the strips were transferred to new tubes containing 50 μL of a solution consisting of 1 μM SARS-COV-2 spike protein receptor binding domain (RBD) in 1×PBS. RBD was produced in house as described. The RBD concentration was optimized for maximum signal output from serial coupling reactions testing protein immobilization at different concentrations (FIG. 12A). Using the Thermomixer, the strips were stirred for 2 hours, then incubated in the same solution overnight (18 hours) without stirring at 4° C. On the third day, the strips were rinsed under a jet of deionized water for 5 seconds on each side, and subsequently incubated at room temperature for 60 minutes in 60 μL of a solution consisting of 0.05% Tween 20 and 5% (w/w) casein in 1×PBS to reduce the non-specific binding of biological material present in serum samples. Finally, the strips were washed under a jet of 0.05% Tween 20 in 1×PBS for 15 seconds on each side. The resulting RBD-functionalized strips were stored in 1×PBS until use.

v. Spectrophotometric IgG Detection

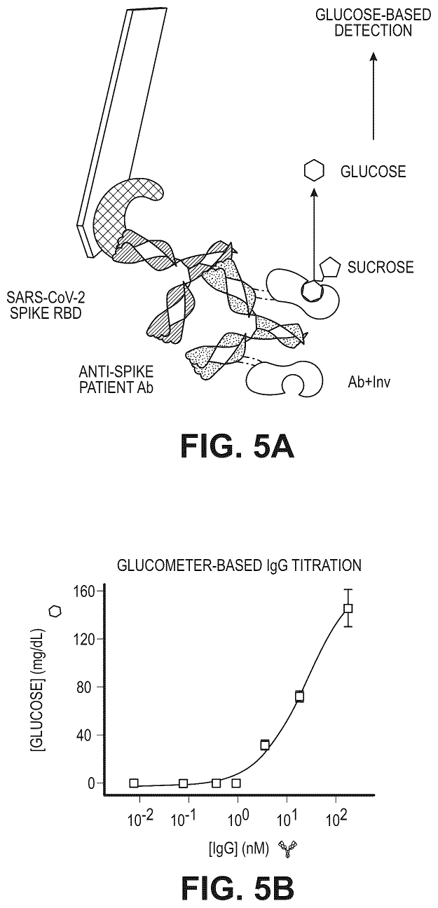

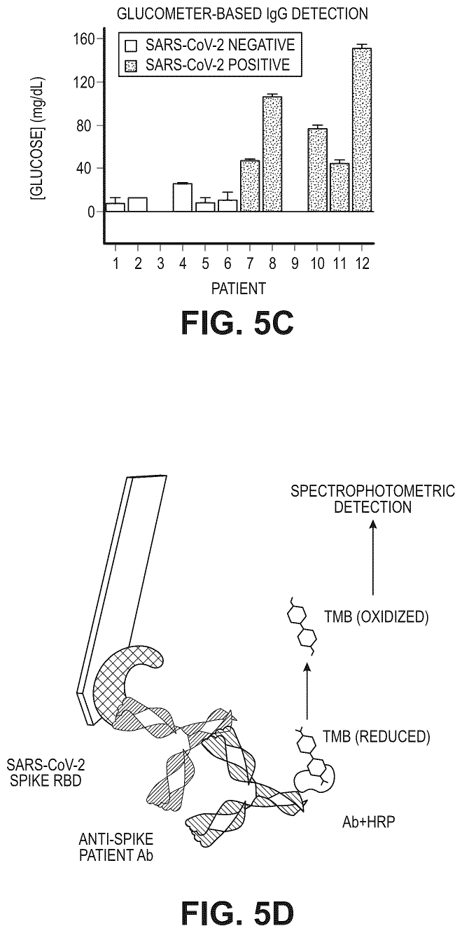

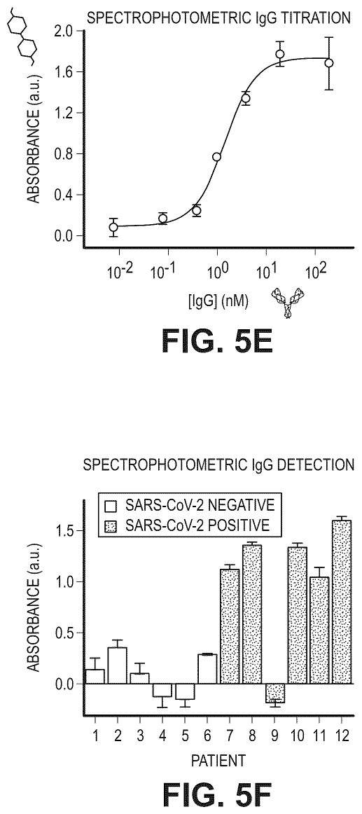

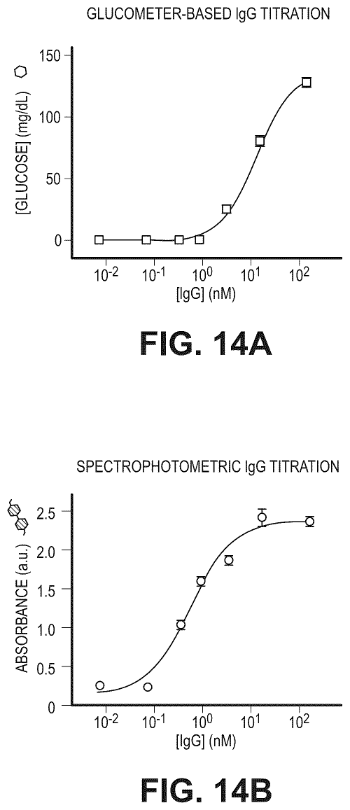

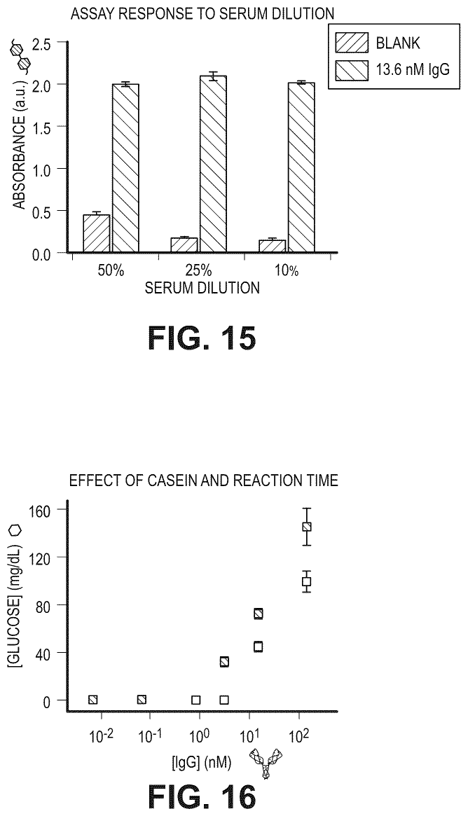

For spectrophotometric immunoassays, SARS-COV-2 spike protein RBD-modified strips were immersed in 50 μL of a 10% commercial human serum solution prepared in 0.05% PBS-Tween 20 and 5% (w/w) casein, which contained titrations of commercial human monoclonal anti-SARS-COV-2 spike protein RBD antibody (ab273073, Abcam) or clinical samples for 30 minutes. Next, the SARS-COV-2-targeted antibody-bound strips were rinsed with PBS-Tween 20 solution (see SI Appendix) for 15 seconds each side, and incubated in 50 μL of a 0.13 μM polyclonal anti-hIgG-HRP (Abcam) solution prepared in 0.05% PBS-Tween 20 and 5% (w/w) casein for 30 minutes to allow for binding of the antibody-enzyme fusion protein to captured hIgG antibodies. Strips were rinsed one last time for 15 seconds with the PBS-Tween 20 solution, and dipped for 5 minutes in 1×PBS. Last, the strips were dipped in 50 μL of a commercial TMB/H₂O₂ solution (Sigma) and allowed to react for 10 minutes so that oxidation of TMB by bound HRP could occur. The reaction was halted by adding 10 μL of a 0.5 M H₂SO₄ solution. The resulting absorbance at 450 nm was measured using a NanoPhotometer® NP80. After optimizing the loading of RBD on the strips and the concentration of the HRP-modified secondary antibody (FIG. 12A-12C), spectrophotometric calibrations were performed in 1×PBS (FIG. 14A). Before shifting to more complex samples, the dilution factor for the clinical samples was optimized using commercially available human serum (BioIVT, catalog #HUMANSRM 0000351) at various concentrations, and diluting the samples in PBS with 0.05% Tween-20 and 5% casein (FIG. 15). Although the results showed that the designed spectrophotometric assay leads to relative low background signals even in 50% serum, to optimize the number of tests done with the available clinical samples, it is decided to dilute serum samples were diluted to a 10% concentration. IgG standard solutions were prepared in 10% serum to obtain the spectrophotometric titration curve (FIG. 5E) and to quantify the IgG present in clinical samples (FIG. 5F).

vi. Glucometer-Based IgG Detection

For the glucometer-based immunoassays, casein was not included in the incubation buffer, as the electrochemical response and the affinity of the secondary antibody decreased, hindering the quantification of low IgG levels (FIG. 16). SARS-COV-2 spike protein RBD-modified strips were incubated with samples containing either commercial human monoclonal anti-SARS-COV-2 spike protein RBD antibody (ab273073, Abcam) or IgG present in clinical samples in a similar fashion to in the spectrophotometric assay to allow antiviral antibodies to bind. Strips were then rinsed with 0.05% PBS-Tween 20 for 15 seconds on each side, and incubated in 50 μL of an optimized concentration of LC15 (0.1 μM prepared in 0.05% PBS-Tween 20) for 30 minutes to allow for binding of the antibody-invertase fusion protein to captured hIgG antibodies. Strips were rinsed one last time for 15 seconds with the PBS-Tween 20 solution, and dipped for 5 minutes in 1×PBS. Last, strips were immersed in 50 μL of a 0.1 M sucrose solution prepared in buffer for 60 minutes to allow for conversion by the bound invertase. After the enzymatic reaction, 5 μL of each solution was pipetted into a plastic Petri dish, and the glucose concentration was determined using a StatStrip medical-grade glucometer (Nova Biomedical). This glucometer model is used as the standard-of-care for glucose detection in the Johns Hopkins Hospital Point-of-Care Laboratory. Stat Strip Xpress glucose strips from the same company were employed (lot 0320002249). All measurements were calibrated against a glucose control solution, also provided by Nova Biomedical.

vii. Blinded Testing of Clinical Samples

All clinical samples used in this Example were acquired under approval by the Institutional Review Board of the Johns Hopkins University School of Medicine, protocol IRB00250000. The parent studies were IRB00247886, IRB00250798, and IRB00091667. The samples were collected from the Johns Hopkins Hospital, with consent from healthy individuals and infected patients, and stripped from all HIPPA-defined identifiable information. The parent studies were conducted according to the ethical standards of the Helsinki Declaration of the World Medical Association. This report includes an analysis of stored samples and data from those studies.

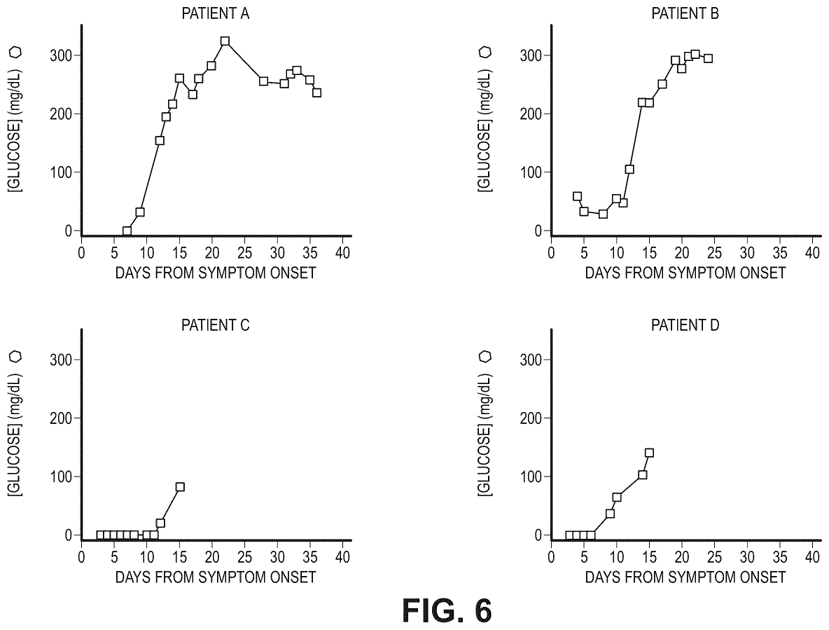

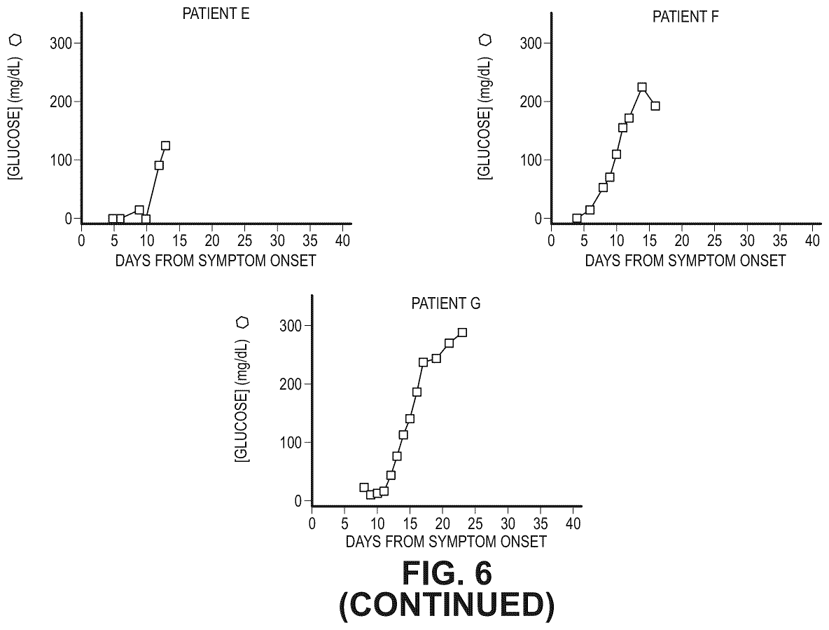

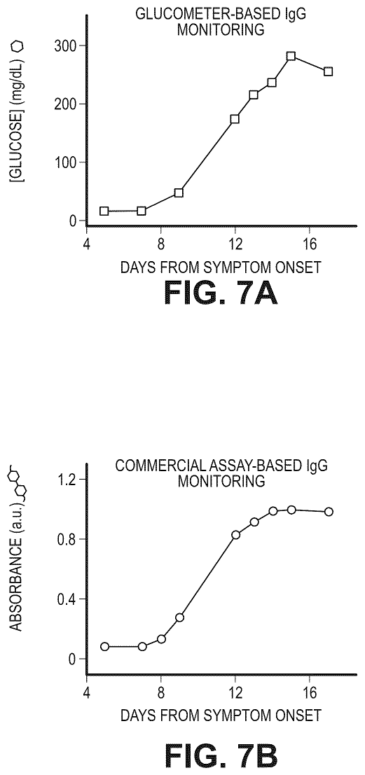

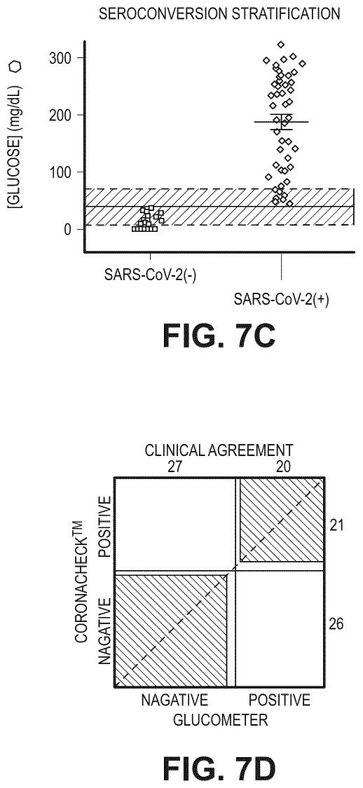

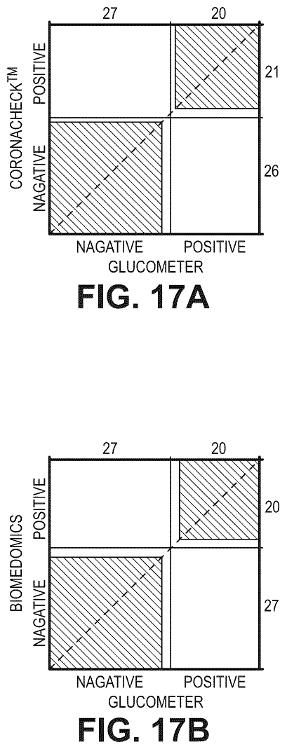

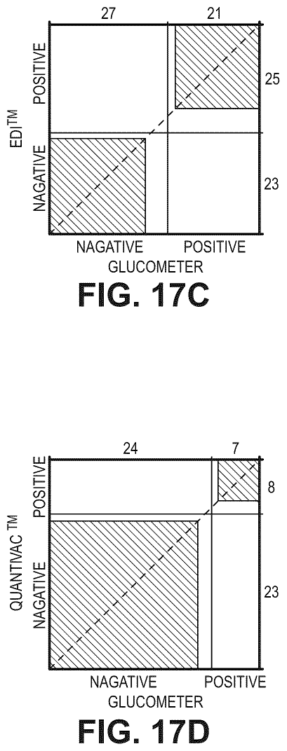

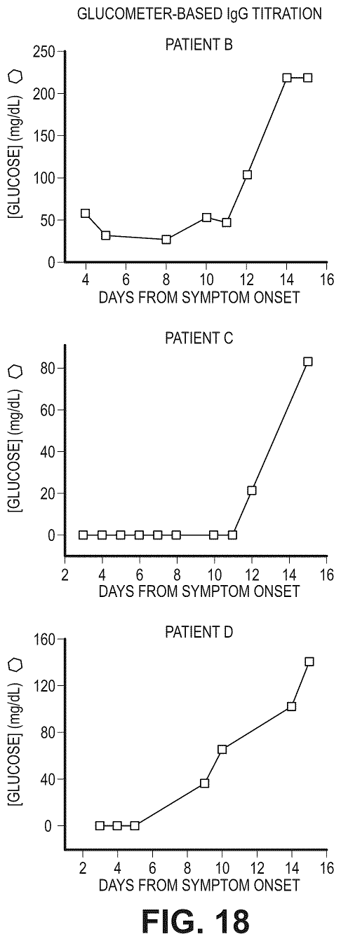

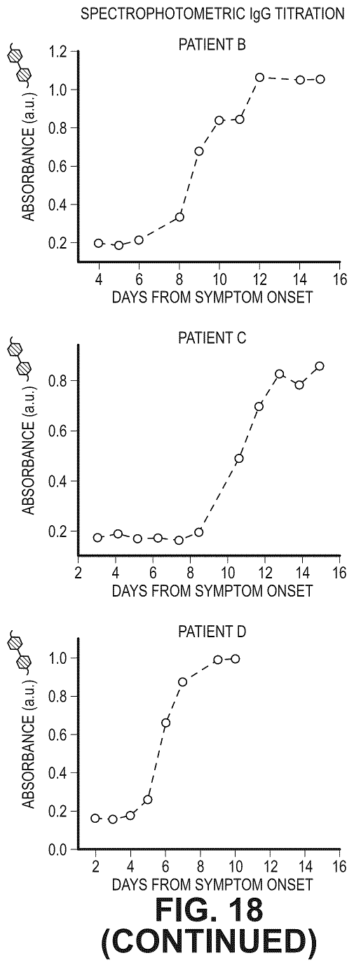

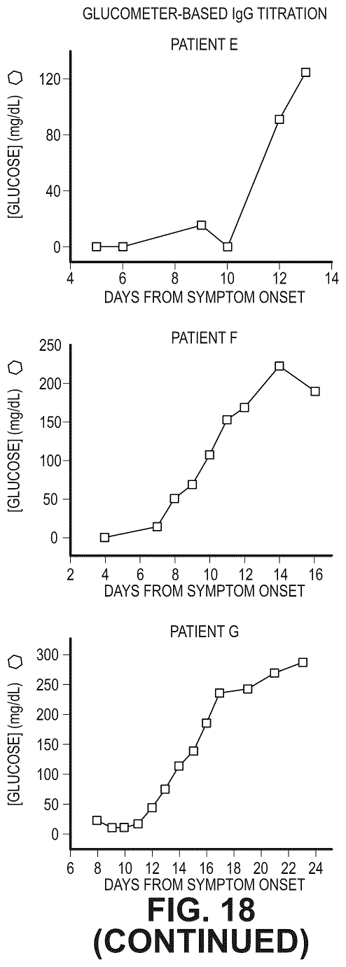

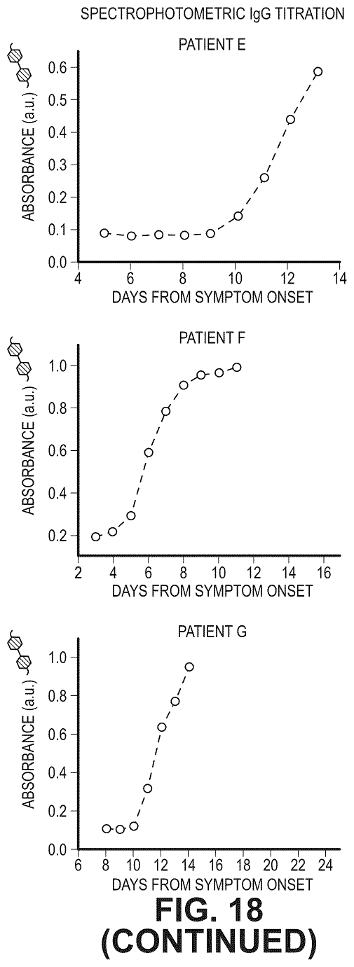

Clinical serum samples were diluted to a final 10% concentration and either spectrophotometric or glucometer-based detection was performed, as described above. All experiments involving the handling of clinical samples from COVID-19 patients were performed inside a class II laminar flow biosafety cabinet (Nuaire), and with approval of the Johns Hopkins University Institutional Biosafety Committee, under registrations #B2004270101 and #P2004200401. The samples delivered to the study team were split in two training sets: TS1 contained a pool of 12 specimens collected at unknown points in time after confirmed infection (via RT-PCR); TS2 contained a pool of 90 longitudinal specimens from 7 patients collected at known time intervals. The study team was blinded to the order and number of specimens from individual patients, and the sample collection times. Four clinical samples of COVID-19 negative patients were also used to establish the threshold shown in FIG. 7C. The cross comparisons shown in FIG. 5A-5F, FIG. 6, FIG. 7A-7D, FIG. 17A-17D and FIG. 18 were only made after revealing study results to the clinical team, who performed clinical RT-PCR and ELISA determinations independently.

### 2. Results

i. Design and Purification of Anti-hIgG-(Invertase) 2 (Ab+Inv) Fusion Proteins

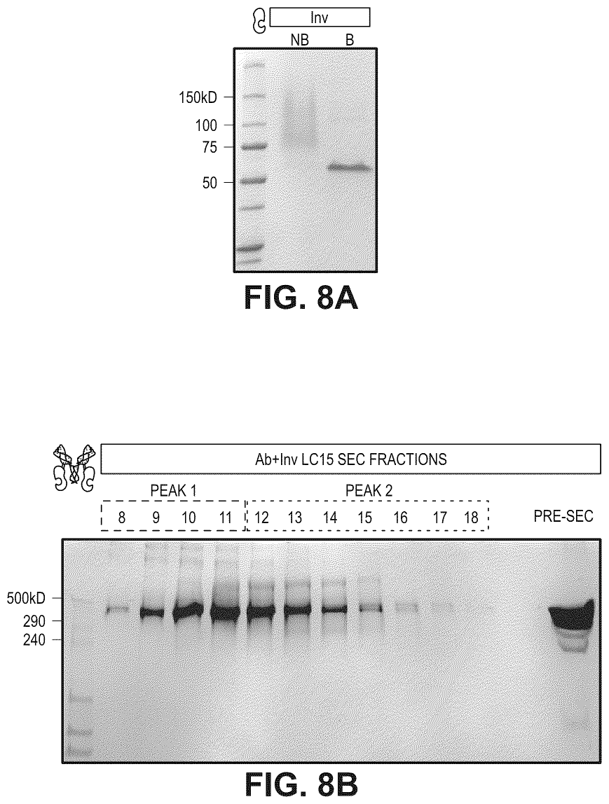

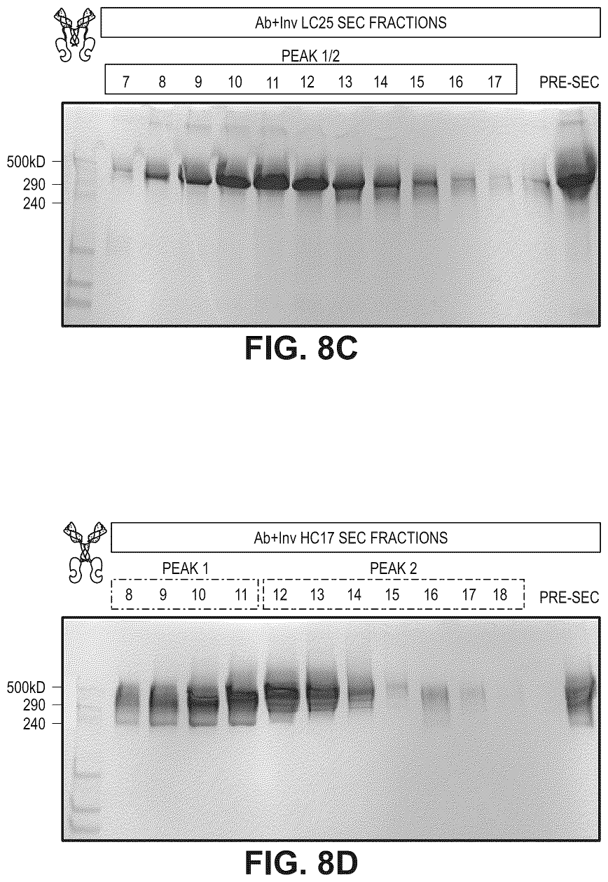

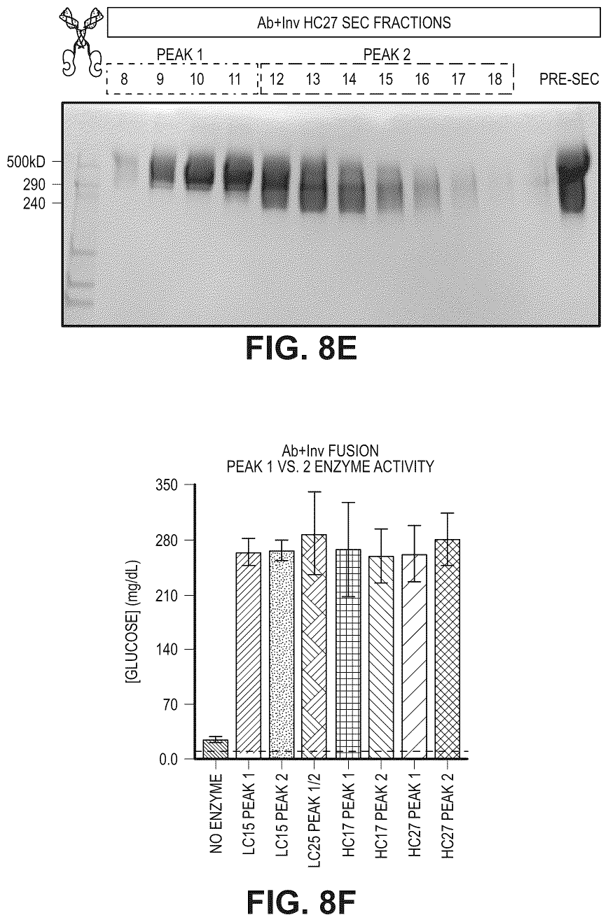

We produced four anti-human IgG-invertase fusion proteins (Ab+Inv, FIG. 2A), that varied in the positioning and length of the peptide linkers connecting the antibody to two molecules of invertase. Fusion proteins were based on the anti-human IgG antibody HP6017, which binds the Fc domain of all human IgG isotypes (Jefferis et al., Immunol. Lett., 1985, 10 (3-4): 223-252 (doi: 10.1016/0165-2478 (85) 90082-3; PMID: 3899923) and Reimer et al., Hybridoma, 1984, 3 (3): 263-275 (doi: 10.1089/hyb.1984.3.263; PMID: 6209201). The four fusion proteins were produced via transient transfection of human embryonic kidney (HEK) 293F cells. The fusion proteins ran slightly larger than their expected sizes (each ˜266 kDa), which was consistent with the observation that unboiled invertase runs larger than its predicted molecular weight of 62 kDa (FIG. 2B, FIG. 8A-E). Accordingly, under reducing conditions, fusion proteins comprising invertase and a single light chain (LC) or heavy chain (HC) ran slightly larger than their predicted molecular weights (84 kDa for LC fusion proteins and 110 kDa for HC fusion proteins), whereas the unfused LC and HC ran as expected (FIG. 2B). All Ab+Inv fusion proteins expressed robustly, with purified protein yields ranging from 2.8 to 4.5 mg per L of transfection, similar to the unfused antibody yield of 3.5 mg/L (FIG. 2C). The four Ab+Inv fusion proteins showed similar profiles by size exclusion chromatography (SEC), though the HC fusion proteins (HC17 and HC27) exhibited a discernable shoulder extending to the left of the main peak (denoted peak 1, FIG. 2D). Moreover, earlier fractions of LC15, coinciding with peak 1, contained greater amounts of aggregate (FIG. 8B). Interestingly, comparison of the pooled peak 1 and peak 2 fractions from the HC and LC15 fusion proteins revealed similar activity (FIG. 8F). Due to reduced aggregation, peak 2 of LC15, HC17 and HC27 was used for all subsequent characterization.

ii. Validation of Ab+Inv Binding to Human IgG

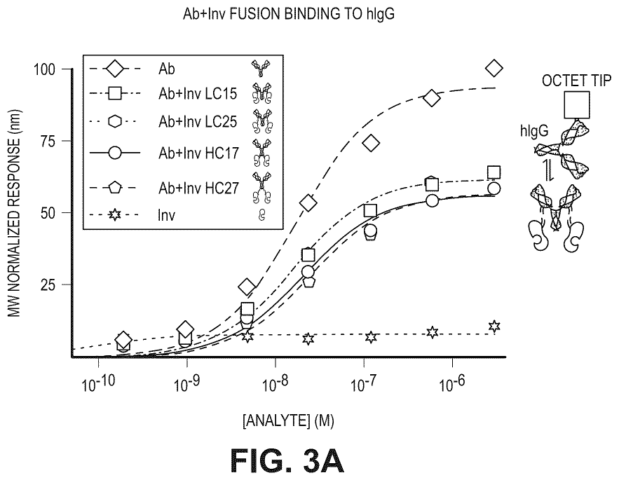

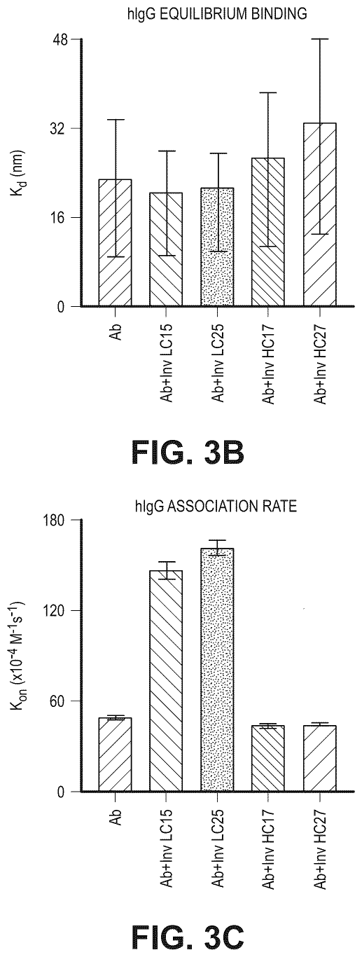

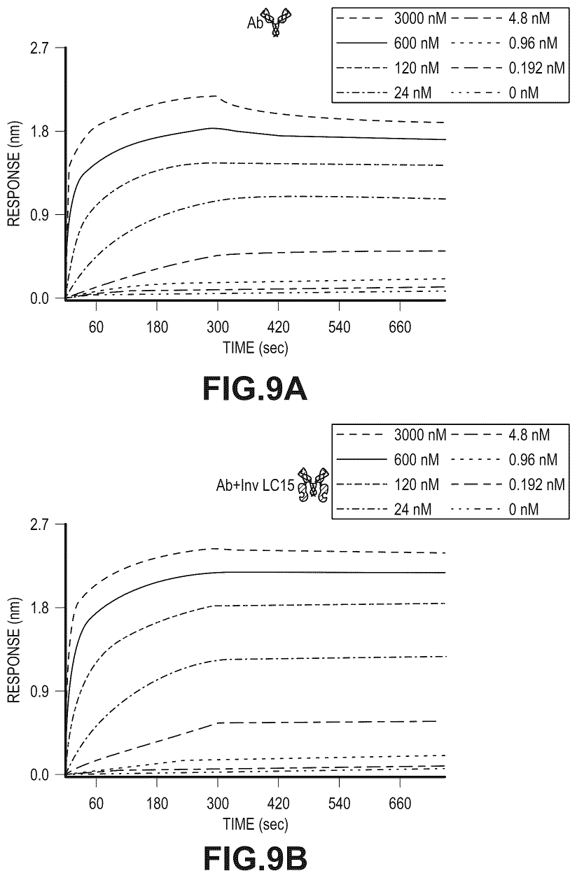

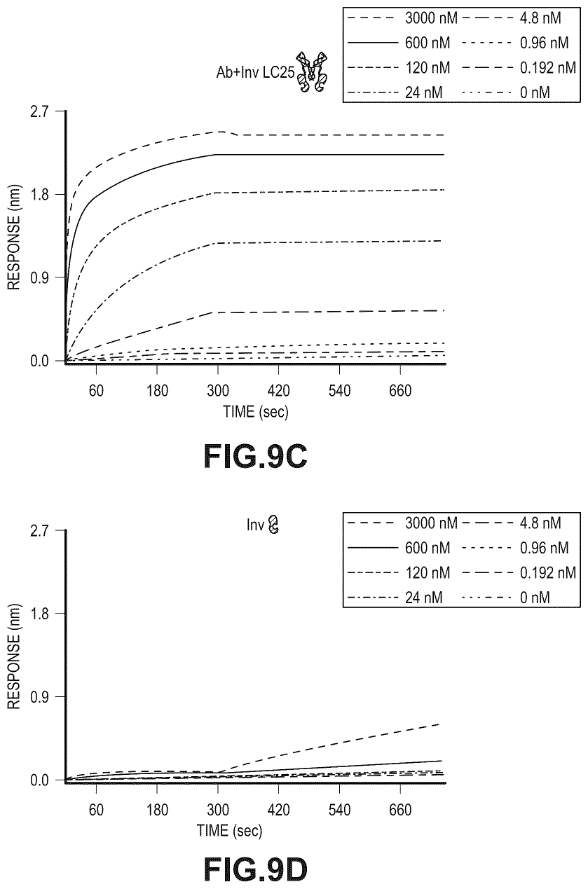

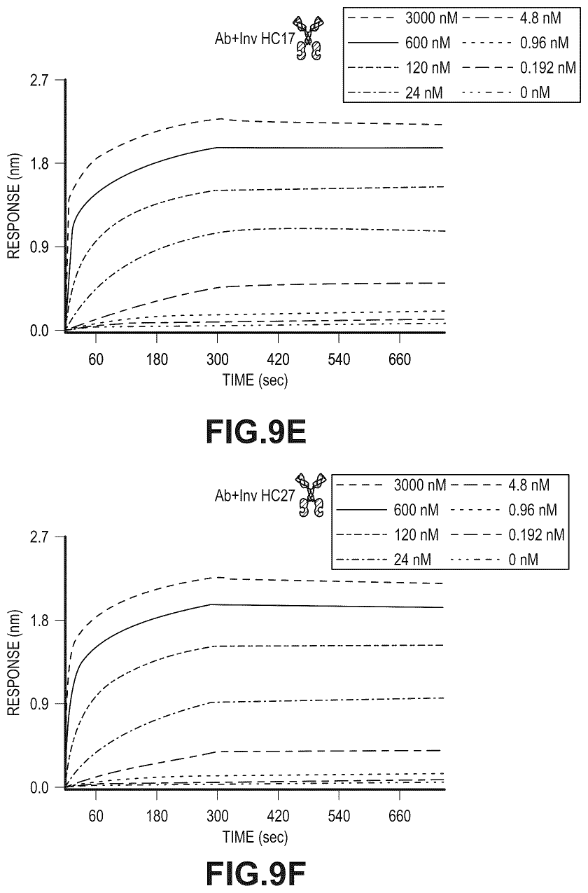

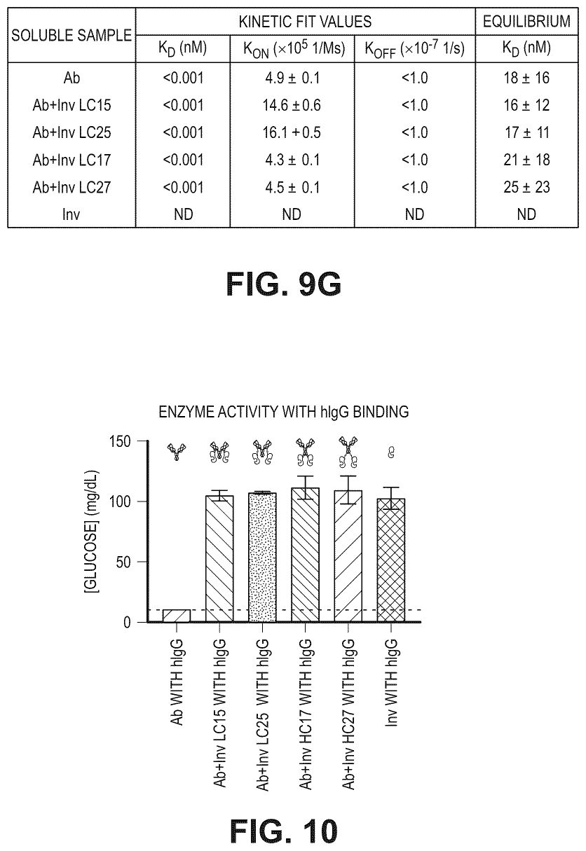

To demonstrate that our Ab+Inv fusion proteins retained binding to the target antigen (i.e., human IgG), bio-layer interferometry studies were performed. The four Ab+Inv fusion proteins showed similar human IgG binding properties relative to what was observed for the unfused antibody, with LC15 and LC25 showing a marginally higher affinity (FIG. 3A, FIG. 3B, Table 1). The Ab+Inv fusion proteins showed slightly lower maximal binding levels (Eₘₐₓ) compared to the unfused antibody, presumably due to steric effects resulting from the increased size of the invertase fusion. Analysis of kinetic binding parameters further revealed LC15 and LC25 had significantly higher association rates compared to both the unfused antibody and the HC fusion proteins (FIG. 3C, FIG. 9A-9G, Table 1). While the binding properties of all four Ab+Inv fusion proteins were very similar, these data suggested that the LC fusion proteins had slightly superior binding activity compared to the HC fusion proteins.

iii. Validation of Ab+Inv Catalytic Activity

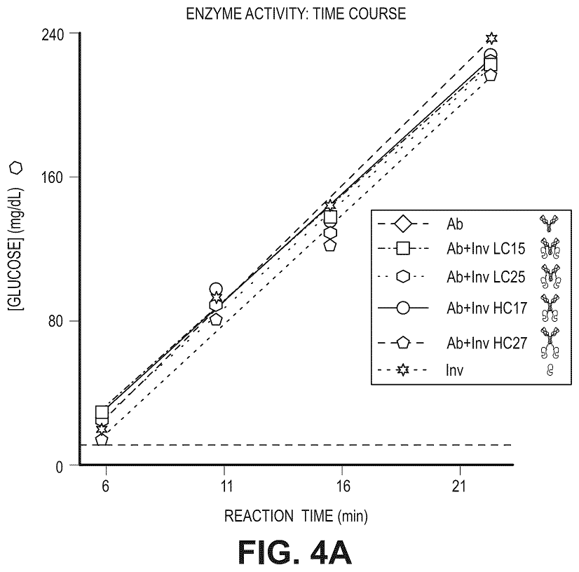

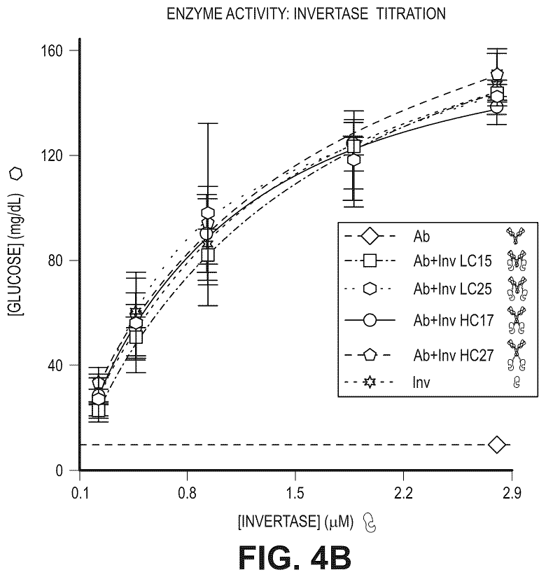

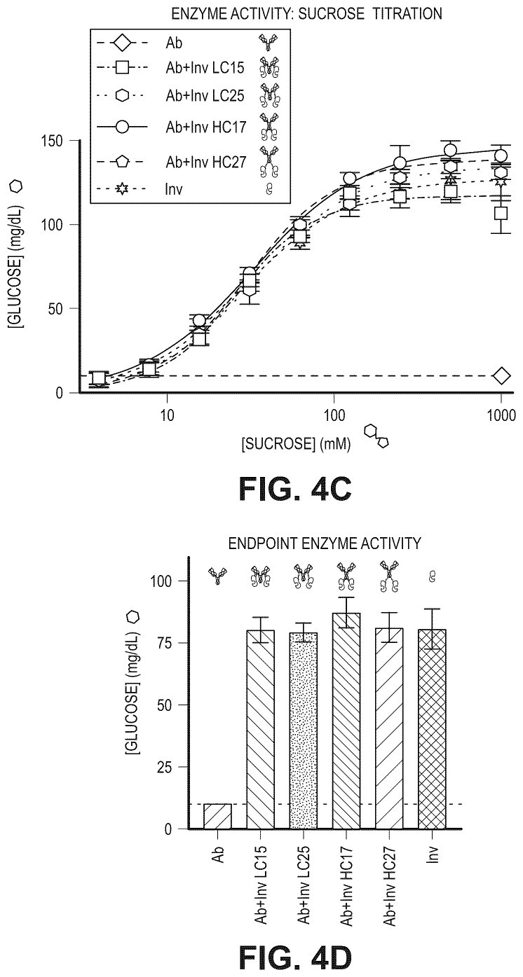

To determine whether our Ab+Inv fusion proteins preserved the catalytic activity of the component invertase enzyme, we implemented glucose conversion assays using a commercial glucometer. We compared the catalytic activity of the four Ab+Inv fusion proteins in solution by measuring the extent of glucose production after incubating the proteins with specified concentrations of sucrose. The four Ab+Inv fusion proteins achieved nearly identical enzymatic activity to the unfused invertase in time-course experiments (FIG. 4A), enzyme titrations (FIG. 4B), substrate (sucrose) titrations (FIG. 4C), and endpoint activity measurements (FIG. 4D). Enzymatic activity was not affected by concurrent binding to hIgG, as was demonstrated by endpoint experiments performed with a 2× molar excess of hIgG (FIG. 10). While the overall enzymatic activities for each of the four Ab+Inv fusion proteins were very similar, LC25 exhibited the greater variability for measurements near the middle of the dynamic range for the enzyme titrations (FIG. 4B).

iv. Development and Deployment of an Anti-SARS-COV-2 Antibody Detection Assay

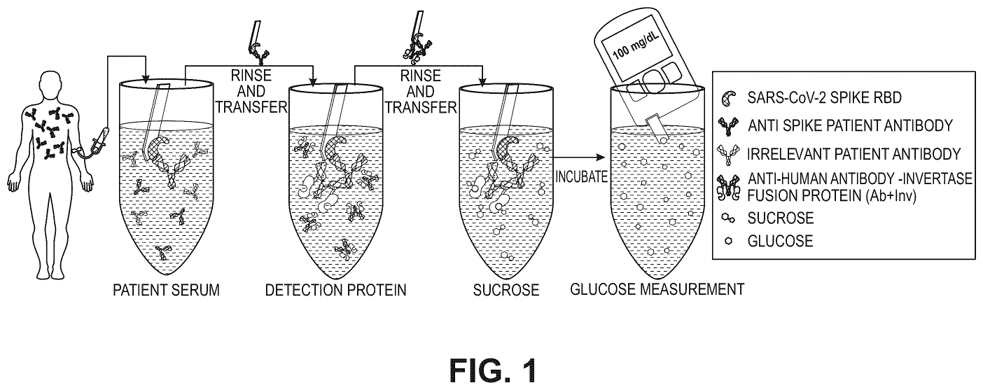

To demonstrate the diagnostic potential for our Ab+Inv fusion proteins, we sought to establish that they could accurately differentiate between patient samples that did or did not contain SARS-COV-2-specific antibodies via a strip-based assay that would be amenable to point-of-care use (FIG. 1). To build the assay, we designed plastic test strips (FIG. 11A-11B) that fit snugly inside 1.5 mL microcentrifuge tubes. The strips were fabricated using a CO₂ laser cutter from 2 mm thick poly(methyl methacrylate) sheets (example cuts shown in FIG. 11A-11B). We coated the strips with established antifouling hydrogel technology to minimize non-specific protein binding from patient samples to the surface (28). The hydrogel was then functionalized with the receptor binding domain (RBD) of the SARS-COV-2 spike protein via covalent coupling using carbodiimide chemistry. We established the antigen loading that provided the highest assay sensitivity via spectrophotometric tests in phosphate-buffered saline (PBS, FIG. 12A-12C), consisting of HRP-modified, ELISA-validated anti-spike antibodies and tetramethylbenzidine (TMB)/hydrogen peroxide solutions. The resulting antigen-loaded strips can be stored in buffer at 4° C. and are amenable for use in quantifying anti-SARS-COV-2 antibodies in patient samples.

Our electrochemical immunoassay consisted of exposing the test strips to a series of binding and washing steps to ultimately produce a solution containing glucose in direct proportion to the number of anti-SARS-COV-2 antibodies present in each sample (FIG. 1, FIG. 5A). Briefly, we first immersed the strips in either control or infected patient samples for 30 minutes to allow anti-SARS-COV-2 spike protein RBD antibodies to bind, followed by a wash step. We then exposed the strips to Ab+Inv fusion protein solutions for 30 minutes so that the fusion protein would bind to the captured antibody on the strip, also followed by a wash. In the third step, we immersed the strips in 100 mM sucrose solution for 30 minutes to allow invertase to catalyze conversion to glucose, after which we removed the strips and determined the resulting glucose concentration using a commercial glucometer. The exact measurement protocol varied slightly depending on whether we performed control measurements using the spectrophotometric HRP/TMB reporter system, or the invertase/sucrose reporter system, as described in detail in the Methods section.

We first established which of our four Ab+Inv fusion proteins achieved the greatest sensitivity in strip-based immunoassays. To do this, we incubated the strips with primary anti-spike antibody (13.6 nM), followed by exposure to saturating concentrations (0.1 μM) of each of the four Ab+Inv fusion proteins. Our results indicated that, under identical assay conditions, the LC fusion proteins produced about 55% more glucose than the HC fusion proteins (FIG. 13). Because the LC15 fusion protein achieved the highest generation of glucose, we selected this molecule for assay development. We first optimized the concentration of LC15 for the strip assay (FIG. 12B), and subsequently built assay calibrations in PBS to establish the dynamic range of our reporter relative to spectrophotometric detection (FIG. 14). The detection range of both methods compared well, allowing us to proceed to clinical validation trials. For such trials, we accessed confirmed negative and positive patient blood samples from institutional biorepositories. We obtained two blinded training sample sets: TS1 contained 6 confirmed negatives (from pre-pandemic Johns Hopkins Hospital emergency department patients), and 6 confirmed positives (from potential convalescent plasma donors who had prior RT-PCR confirmation of SARS-COV-2 infection) (29). TS2 consisted of 90 longitudinal samples from 7 hospitalized SARS-COV-2 RT-PCR-confirmed patients with known date of symptom onset (30). The antibody titers of both training sets were unknown to our study team until we performed a crossed examination against commercial ELISA measurements.

Using the LC15 fusion protein as reporter, we sought to determine whether our glucometer-based immunoassays achieved similar discrimination of negative and positive samples from training set TS1 relative to analogous colorimetric assays. To develop the assay framework to test this, we first comparatively evaluated the performance of the LC15/sucrose (FIG. 5A) and HRP/TMB (FIG. 5D) reporter systems built from strip-based assays using 10% commercial SARS-COV-2 negative patient sera spiked with increasing concentrations of commercial anti-SARS-COV-2 spike protein RBD IgGs. We observed strong assay responses at this dilution of the patient serum samples (using the spectrophotometric test, FIG. 15); thus, a serum dilution of 10% was used for all subsequent assays. We used casein in all the strip-based assays involving human serum to prevent non-specific protein binding to the test strips, by incubating the strips into a casein solution at a concentration of 5% (w/w) for 1 hour. However, for the glucometer-based detection, we did not add casein to the human serum samples as is common practice in the field, because we observed a about 2-fold improvement in the signal output of the sensor in the absence of this reagent (FIG. 16). We also optimized the strip incubation time with the sucrose solution. Based on our findings, incubation of the strips in serum without casein for binding to patient antibodies and in sucrose solution for 60 minutes during detection were selected. The resulting dose-response curves indicated that the HRP/TMB reporter achieved higher sensitivity (Kp about 2 nM, FIG. 5E) and plateaued at lower concentrations of anti-SARS-COV-2 spike protein RBD IgG (about 13.6 nM) compared to the LC15/sucrose reporter system (K_{D} about 35 nM, plateau at >150 nM, FIG. 5B). These results were not unexpected, as the catalytic rate of HRP is approximately two-fold faster than that of the invertase in LC15. Despite this discrepancy in sensitivity for detecting pure IgG, when we challenged both systems with TS1 samples, they exhibited comparable capacity to distinguish between all confirmed negative and positive specimens (FIG. 5C vs. FIG. 5F). Notably, the apparently false negative data obtained for patient 9 was later confirmed to be a true negative via commercial ELISA assays (Epitope Diagnostics (EDI™) COVID-19), and the sample corresponds to a convalescent patient with unusually low antibody titers.

We next examined whether the LC15 fusion protein would enable accurate glucometer-based monitoring of immune responses in longitudinal samples. To this end, we exposed our test strips to samples from training set TS2 and compared results against the clinically used EDI™ COVID-19 and CoronaCheck™ immunoassays. Note that the EDIT assay employs the nucleocapsid of SARS-COV-2 as the target antigen, whereas the CoronaCheck™ assaym like our glucometer-based immunoassays, employs the RBD protein. Our glucometer-based measurements showed an onset of anti-SARS-COV-2 IgGs at about 10 days after the first record of symptoms across patient samples (FIG. 6). In addition, for the three sample sets with a sufficient number of measurement points to identify a plateau in antibody responses, we characterized such a plateau at about 20 days post infection. This finding was in close agreement with results from commercial antibody assays (FIG. 17, FIG. 18) and demonstrated superior seroconversion resolution over, for example, lateral flow immunoassays (30, 31). Moreover, in a side-by-side comparison of LC15/sucrose (FIG. 7A) and EDI™ COVID-19 (FIG. 7B) measurements using the same patient samples (TS2), the resulting time course profiles were indistinguishable from one another. These results highlight the potential value that our fusion protein reporter and glucometer-based immunoassay have in monitoring the development and maintenance of immunity temporally, across populations, without the need for spectrophotometric or other optical equipment.

Two critical figures of merit for new immunoassays are the positive (PPA) and negative percent agreements (NPA) relative to benchmark commercial immunoassays. To determine the PPA and NPA for our glucometer-based assay, we first established a positive seroconversion threshold based on the average response of our assay to confirmed negative specimens (FIG. 7C). Specifically, we measured the output of our assay when challenged with four pre-pandemic samples from hospitalized patients plus one commercial sample pooled from 10 healthy donors. We established our seroconversion threshold by computing the mean and standard deviation from those measurements, 39±32 mg/dL (black line and shaded areas, respectively, in FIG. 7C), to effectively set the background level above which all measurements were considered to be seroconversion positive. Applying this cutoff to our measurements over all longitudinal samples, we deemed 64% (49 samples) to be positive and 36% (27 samples) to be negative. The accuracy of this determination can be better demonstrated by directly comparing glucometer-based immunoassay determinations relative to CoronaCheck™ measurements. The resulting agreement chart (FIG. 7D), reported a PPA=96% (26/27*100) and NPA=95% (20/21*100), demonstrating again that our glucometer-based immunoassays achieve similar performance relative to benchmark spectrophotometric assays currently in clinical use.

### 3. Discussion

We report here a novel fusion protein comprising anti-hIgG and two invertase molecules, which can be used as an electrochemical reporter for rapid and robust glucometer-based immunoassays. By virtue of being expressed as a single protein from a eukaryotic cell line, our fusion protein overcomes purification and heterogeneity challenges related to the post-expression chemical coupling of enzymatic reporters to detection antibodies. Specifically, we were able to express four anti-human antibody-invertase fusion proteins (Ab+Inv), all of which were successfully expressed in mg/L yields (FIG. 2C) and retained the binding affinity and catalytic activity of the respective unconjugated parent proteins (FIG. 3A-3C, FIG. 4A-4D). The Ab+Inv fusion protein that showed the best functional activity, denoted LC15, enables the possibility to perform immunoassays using widely available glucometers as detectors, obviating the need for expensive and specialized spectrophotometric or optical instrumentation, and reducing the level of technical skill required to perform the test. Moreover, because LC15 binds to the Fc region of all human IgG isotypes, the protein can be immediately used as a secondary antibody-reporter for a vast range of antibody assays, in a similar fashion as the benchmark HRP/TMB system.

To illustrate the ability of LC15 to support accurate antibody measurements in clinical specimens, we developed a plastic-strip based immunoassay that quantifies anti-SARS-CoV-2 antibodies as a model case for our glucose-based detection system. We then validated our platform by performing glucometer-based antibody assays using two different clinical blood sample training sets, one consisting of pre-pandemic negative samples and samples from SARS-CoV-2 positive patients, and a second set consisting of longitudinal samples from hospitalized, COVID-19-confirmed patients. Based on studies using both training sets, our glucometer-based immunoassays performed comparably against either an identical HRP/TMB-based test performed in our lab (FIG. 5A-5F), or commercial immunoassays that are currently in clinical use (FIG. 6, FIG. 7A-7D). Our results demonstrate remarkable accuracy and agreement with commercially-available immunoassays. We also note that optimization of the anti-hIgG antibody in LC15 through affinity maturation could further improve the sensitivity and performance of our assay. With increased sensitivity, it is likely that our diagnostic will be translatable for use with other types of patient samples, such as nasal/pharyngeal swab (32) or saliva.

The Ab+Inv fusion protein LC15 represents an emerging class of reporter proteins. While antibodies and other binding molecules have previously been chemically conjugated to invertase, to our knowledge, this is the first example wherein invertase has been attached directly through genetic fusion. Importantly, the yields observed for Ab+Inv LC15 were very similar to the yield for the unfused antibody (FIG. 2C), indicating that the cost of large-scale production of the fusion would be similar to that for the antibody alone. While LC15 did appear to have more aggregate in peak 1, this aggregate did not impact binding or enzymatic activity, and these aggregates could likely be eliminated through optimization of transfection conditions. Encouragingly, LC15 eluted as a monodisperse peak via SEC, suggesting high-purity manufacturing with limited attrition will be feasible. Moreover, as invertase is genetically fused to the antibody, we have eliminated additional chemical conjugation steps, which can be laborious and inconsistent, and require additional purification to remove unconjugated protein, as well as quality confirmation. Furthermore, chemical conjugation methods are inherently stochastic, meaning that the number of invertase molecules conjugated to antibody will unavoidably vary, whereas in our approach the stoichiometry is fixed, which ensures high consistency between batches.

More broadly, this study represents one of the first instances in which a full-length antibody is genetically fused to an enzyme for the purposes of detection. The vast majority of previous work has generated scFv-enzyme fusion proteins (33-38), or fragment antigen-binding (Fab)-enzyme fusion proteins (39, 40). scFvs and Fabs contain only one antigen binding site per molecule, eliminating the avidity advantage afforded by the two binding sites in a full-length antibody. To our knowledge, only one platform has been developed to produce enzymes that are genetically fused to full-length antibodies, specifically a fusion protein scaffold linking an enzyme to IgM, yielding a highly multimeric antibody-enzyme pentamer (41). While the pentameric structure of IgM provides a dramatic avidity enhancement, naturally-occurring IgMs generally have very low affinity compared to IgGs, and are notoriously difficult to produce (42, 43). As such, our full-length IgG formulation leverages the multimeric avidity advantage while avoiding losses in yield, making it an ideal candidate for large-scale production.

The low technical requirements and low production cost for our Ab+Inv fusion protein will allow the proposed glucometer-based diagnostic platform to be employed for testing of far greater numbers of people on the global scale, including those who do not have access to medical facilities with advanced testing capabilities. It will also empower serial testing, which combined with the number and diversity of people being tested, will provide needed high-quality data to impart a clear and detailed understanding of the longevity of immune protection that is provided from vaccination and natural infection, as well as protection against emerging variants of concern. Importantly, this assay can be easily modified to specifically test for antibodies against variant RBDs by simply replacing the original RBD with the variant RBD containing the relevant mutations. Moreover, replacing the SARS-COV-2 RBD with another antigen from another infectious disease, a cancer diagnostic antigen, or a self-antigen associated with autoimmune diseases would allow this diagnostic to be implemented as a test for immunity to a large number of medical conditions, and for longitudinal monitoring of disease control or progression. Altogether, the results of this study combined with the modularity of this technology highlight the potential value of our glucometer-based antibody detection approach for population-scale monitoring of immune responses to address the COVID-19 pandemic and a host of other biomedical applications. Further device development efforts will focus on simplifying the detection scheme so the fusion protein and other reagents can be integrated into a portable, user-friendly, point-of-need detection platform.

### Example 2. Recombinant Antibodies Comprising Anti-Human IgG-Invertase Fusion Proteins for Infectious Disease Screening

The recombinant antibodies comprising anti-human IgG-invertase fusion proteins described in Example 1, or any equivalents thereof (collectively, “the recombinant antibody fusion proteins of the disclosure”), can be used as a reporter to screen for a panel of infectious diseases via the presence of human antibodies against pathogen-specific antigens. To do so, a test strip-based protocol can be conducted as described in Example 1, but with substitution of the SARS-CoV-2 RBD with an alternative pathogen associated with an infectious disease. Alternatively, a multi-well plate, such as a 96-well plate, may be functionalized to have a different pathogenic antigen per well. Each pathogenic antigen is immobilized or absorbed onto the well. The wells are then exposed to patient samples (e.g., urine, saliva, serum) and incubated for a period to allow any existing patient antibodies to bind the antigens. After washing the plates, the wells are filled with a solution of sucrose and the recombinant antibody fusion proteins of the disclosure. The net concentration of glucose generated from sucrose after a period of time (e.g., 30 minutes) is then measured using a glucometer. Any wells containing glucose above background confirm positive seroconversion (positive immune response or positive infection). Development of this technology in multiplex format would be particularly important for rapid and inexpensive dissemination of the platform on the global scale.

### Example 3. Recombinant Antibodies Comprising Anti-Human IgG-Invertase Fusion Proteins for Diagnosis of Autoimmune Diseases or Disorders

In a similar fashion as described in Example 2, a multi-well plate, such as a 96-well plate, is functionalized with a panel of human proteins, peptides, nucleic acids or cells that are known targets for autoimmune diseases. After exposing the wells to patient samples first, and then to the sucrose solution and the recombinant antibody fusion proteins of the disclosure, any wells containing glucose concentration above background confirm positive seroconversion for a given autoimmune disease. The ability to multiplex seroconversion status is critical here since autoimmune diseases are hard to diagnose and often require the presence of multiple antigen positives to confirm disease status.

### Example 4. Recombinant Antibodies Comprising Anti-Human IgG-Invertase Fusion Proteins for Single Point Antibody Measurements or Serial Monitoring of Immunity Over Time

The recombinant antibody fusion proteins of the disclosure can be incorporated into single point antibody measurements or used for serial monitoring of immunity over time as demonstrated in Example 1. For example, a single glucometer can be used to measure the concentration of glucose produced by the recombinant antibody fusion proteins of the disclosure from patient samples taken at different time intervals from the onset of symptoms. Each new measurement would require a different glucose test strip, but the same glucometer is used. Thus, a single device can be used to instantaneously measure human antibody levels against an antigen, and to determine the state of immunity over time. This approach is far superior to benchmark optical strategies that use expensive equipment requiring trained personnel and centralized infrastructure, often leading to sample processing backlogs due to the time it takes to analyze each sample through the same instrument.

### REFERENCES

1. J. Goudsmit, The paramount importance of serological surveys of SARS-COV-2 infection and immunity. Eur J Epidemiol 35, 331-333 (2020).
2. E. S. Theel, et al., The Role of Antibody Testing for SARS-COV-2: Is There One? J Clin Microbiol 58 (2020).
3. Y. C. Bartsch, et al., Discrete SARS-COV-2 antibody titers track with functional humoral stability. Nat Commun 12, 1018 (2021).
4. S. Feng, et al., Correlates of protection against symptomatic and asymptomatic SARS-COV-2 infection. Nat Med (2021) https:/doi.org/10.1038/s41591-021-01540-1 (Nov. 2, 2021).
5. Y. Chen, et al., “Differential antibody dynamics to SARS-COV-2 infection and vaccination” (Immunology, 2021) https:/doi.org/10.1101/2021.09.09.459504 (Nov. 2, 2021).
6. D. S. Khoury, et al., Neutralizing antibody levels are highly predictive of immune protection from symptomatic SARS-COV-2 infection. Nat Med 27, 1205-1211 (2021).
7. J. Wei, et al., Antibody responses to SARS-COV-2 vaccines in 45,965 adults from the general population of the United Kingdom. Nat Microbiol 6, 1140-1149 (2021).
8. F. Amanat, et al., A serological assay to detect SARS-COV-2 seroconversion in humans. Nat Med 26, 1033-1036 (2020).
9. A. Mendrone-Junior, et al., Correlation between SARS-COV-2 antibody screening by immunoassay and neutralizing antibody testing. Transfusion 61, 1181-1190 (2021).
10. H. Oh, H. Ahn, A. Tripathi, A Closer Look into FDA-EUA Approved Diagnostic Techniques of Covid-19. ACS Infect. Dis. 7, 2787-2800 (2021).
11. C. Y.-P. Lee, R. T. P. Lin, L. Renia, L. F. P. Ng, Serological Approaches for COVID-19: Epidemiologic Perspective on Surveillance and Control. Front. Immunol. 11, 879 (2020).
12. B. Porstmann, T. Porstmann, E. Nugel, U. Evers, Which of the commonly used marker enzymes gives the best results in colorimetric and fluorimetric enzyme immunoassays: Horseradish peroxidase, alkaline phosphatase or β-galactosidase? Journal of Immunological Methods 79, 27-37 (1985).
13. T. Gundinger, O. Spadiut, A comparative approach to recombinantly produce the plant enzyme horseradish peroxidase in Escherichia coli. Journal of Biotechnology 248, 15-24 (2017).
14. T. L. Lewis, K. A. Roth, “Immunohistochemical Detection Methods” in Pathobiology of Human Disease, (Elsevier, 2014), pp. 3829-3840.
15. S. K. Vashist, J. H. T. Luong, “Enzyme-Linked Immunoassays” in Handbook of Immunoassay Technologies, (Elsevier, 2018), pp. 97-127.
16. M. Moshe, et al., SARS-COV-2 lateral flow assays for possible use in national covid-19 seroprevalence surveys (React 2): diagnostic accuracy study. BMJ, n423 (2021).
17. T. Lan, J. Zhang, Y. Lu, Transforming the blood glucose meter into a general healthcare meter for in vitro diagnostics in mobile health. Biotechnology Advances 34, 331-341 (2016).
18. Y. Xiang, Y. Lu, Using personal glucose meters and functional DNA sensors to quantify a variety of analytical targets. Nature Chem 3, 697-703 (2011).
19. F. P. Chávez, L. Rodriguez, J. Díaz, J. M. Delgado, J. A. Cremata, Purification and characterization of an invertase from Candida utilis: comparison with natural and recombinant yeast invertases. Journal of Biotechnology 53, 67-74 (1997).
20. Gangadhara, P. Ramesh Kumar, V. Prakash, Influence of Polyols on the Stability and Kinetic Parameters of Invertase from Candida utilis: Correlation with the Conformational Stability and Activity. Protein J 27, 440-449 (2008).
21. J. Joo, et al., A facile and sensitive method for detecting pathogenic bacteria using personal glucose meters. Sensors and Actuators B: Chemical 188, 1250-1254 (2013).
22. L. Li, D. Liang, W. Guo, D. Tang, Y. Zeng, Antibody-invertase Cross-linkage Nanoparticles: A New Signal Tag for Point-of-care Immunoassay of Alpha-fetoprotein for Hepatocellular Carcinoma with Personal Glucometer. Electroanalysis, elan.202100212 (2021).
23. J. Lin, D. Tang, Glucometer-based signal readout for a portable low-cost electrochemical immunoassay using branched platinum nanowires. Anal. Methods 8, 4069-4074 (2016).
24. Y. Xiang, Y. Lu, Portable and Quantitative Detection of Protein Biomarkers and Small Molecular Toxins Using Antibodies and Ubiquitous Personal Glucose Meters. Anal. Chem. 84, 4174-4178 (2012).
25. Q. Wang, et al., Sensitive point-of-care monitoring of cardiac biomarker myoglobin using aptamer and ubiquitous personal glucose meter. Biosensors and Bioelectronics 64, 161-164 (2015).
26. S. Zhang, et al., DNAzyme Amplified Aptasensing Platform for Ochratoxin A Detection Using a Personal Glucose Meter. ACS Appl. Mater. Interfaces 13, 9472-9481 (2021).
27. N. F. Ismail, T. S. Lim, Site-specific scFv labelling with invertase via Sortase A mechanism as a platform for antibody-antigen detection using the personal glucose meter. Sci Rep 6, 19338 (2016).
28. J. Sabaté del Río, O. Y. F. Henry, P. Jolly, D. E. Ingber, An antifouling coating that enables affinity-based electrochemical biosensing in complex biological fluids. Nat. Nanotechnol. 14, 1143-1149 (2019).
29. E. U. Patel, et al., Comparative Performance of Five Commercially Available Serologic Assays To Detect Antibodies to SARS-COV-2 and Identify Individuals with High Neutralizing Titers. J Clin Microbiol 59 (2021).
30. S. E. Conklin, et al., Evaluation of Serological SARS-COV-2 Lateral Flow Assays for Rapid Point-of-Care Testing. J Clin Microbiol 59 (2021).
31. J. D. Whitman, et al., “Test performance evaluation of SARS-COV-2 serological assays” (Infectious Diseases (except HIV/AIDS), 2020) https:/doi.org/10.1101/2020.04.25.20074856 (Nov. 2, 2021).
32. L. Li, et al., Analysis of viral load in different specimen types and serum antibody levels of COVID-19 patients. J Transl Med 19, 30 (2021).
33. W. Yang, et al., Application of methyl parathion hydrolase (MPH) as a labeling enzyme. Anal Bioanal Chem 390, 2133-2140 (2008).
34. N. Kobayashi, et al., Immunoenzymometric Assay for a Small Molecule, 11-Deoxycortisol, with Attomole-Range Sensitivity Employing an scFv-Enzyme Fusion Protein and Anti-Idiotype Antibodies. Anal. Chem. 78, 2244-2253 (2006).
35. B. Erdag, et al., Cloning of anti-HBsAg single-chain variable fragments from hybridoma cells for one-step ELISA. Biotechnology & Biotechnological Equipment 31, 964-973 (2017).
36. Y. Sasajima, et al., Expression of antibody variable region-human alkaline phosphatase fusion proteins in mammalian cells. Journal of Immunological Methods 361, 57-63 (2010).
37. K. M. Venisnik, Bifunctional antibody-Renilla luciferase fusion protein for in vivo optical detection of tumors. Protein Engineering Design and Selection 19, 453-460 (2006).
38. R. J. Kerschbaumer, S. Hirschl, C. Schwager, M. Ibl, G. Himmler, pDAP2: a vector for construction of alkaline phosphatase fusion-proteins. Immunotechnology 2, 145-150 (1996).
39. P. Ritthisan, T. Ojima-Kato, J. Damnjanović, T. Kojima, H. Nakano, SKIK-zipbody-alkaline phosphatase, a novel antibody fusion protein expressed in Escherichia coli cytoplasm. Journal of Bioscience and Bioengineering 126, 705-709 (2018).
40. A. Mori, T. Ojima-Kato, T. Kojima, H. Nakano, Zipbodyzyme: Development of new antibody-enzyme fusion proteins. Journal of Bioscience and Bioengineering 125, 637-643 (2018).
41. C. Han, M. Ihara, H. Ueda, Expression of an antibody-enzyme complex by the L-chain fusion method. Journal of Bioscience and Bioengineering 116, 17-21 (2013).
42. A. Mader, V. Chromikova, R. Kunert, Recombinant IgM expression in mammalian cells: A target protein challenging biotechnological production. ABB 04, 38-43 (2013).
43. V. Chromikova, A. Mader, W. Steinfellner, R. Kunert, Evaluating the bottlenecks of recombinant IgM production in mammalian cells. Cytotechnology 67, 343-356 (2015).
44. K. Luly, et al., “Poly(beta-amino ester) s as high-yield transfection reagents for recombinant protein production” (Preprints, 2021) https:/doi.org/10.22541/au.161822876.60446320/v1 (Aug. 20, 2021).
45. L. Li, et al., Analysis of viral load in different specimen types and serum antibody levels of COVID-19 patients. J Transl Med 19, 30 (2021). https:/doi.org/10.1186/s12967-020-02693-2

While the foregoing disclosure has been described in some detail by way of illustration and example for purposes of clarity and understanding, it will be clear to one of ordinary skill in the art from a reading of this disclosure that various changes in form and detail can be made without departing from the true scope of the disclosure and may be practiced within the scope of the appended claims. For example, all the protein constructs, methods, and/or component features, steps, elements, or other aspects thereof can be used in various combinations.

Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure also includes embodiments in which more than one, or the entire group members are present in, employed in, or otherwise relevant to a given product or process. Furthermore, it is to be understood that the disclosure encompasses all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. Where elements are presented as lists, (e.g., in Markush group or similar format) it is to be understood that each subgroup of the elements is also disclosed, and any element(s) can be removed from the group. It should be understood that, in general, where embodiments or aspects of the disclosure, is/are referred to as comprising particular elements, features, etc., certain embodiments or aspects consist, or consist essentially of, such elements, features, etc. For purposes of simplicity those embodiments have not in every case been specifically set forth in so many words herein. It should also be understood that any embodiment or aspect of the disclosure can be explicitly excluded from the claims, regardless of whether the specific exclusion is recited in the specification.

All patents, patent applications, websites, other publications or documents, accession numbers and the like cited herein are incorporated by reference in their entirety for all purposes to the same extent as if each individual item were specifically and individually indicated to be so incorporated by reference.

1. A method of detecting an antibody to an antigenic polypeptide in a sample, the method comprising:
a) contacting the sample with the antigenic polypeptide under conditions sufficient for the antibody to bind the antigenic polypeptide to produce a bound antibody;
b) contacting the bound antibody with at least one recombinant antibody fusion protein to produce a binding complex, wherein the at least one recombinant antibody fusion protein is capable of binding to a human immunoglobin (IgG) and has an invertase catalytic activity of converting at least one substrate into at least one detectable product;
c) contacting the binding complex with the at least one substrate under conditions sufficient to convert the at least one substrate into the at least one detectable product; and
d) detecting the presence or absence of the at least one detectable product in the sample, wherein detecting the presence of the at least one detectable product in the sample indicates that the sample contains the antibody to the antigenic polypeptide,
optionally wherein the method further comprises obtaining the sample from the subject, and optionally wherein the sample is an oral fluid, nasal fluid, saliva, blood, plasma, or serum.

2. The method of claim 1, wherein the antigenic polypeptide is:
a) a polypeptide of an infectious pathogen;
b) a polypeptide of a severe acute respiratory syndrome coronavirus-2 (SARS-COV-2); or
c) a polypeptide of human origin.

3-6. (canceled)

7. A method of diagnosing an infection of a pathogen in a subject in need thereof, comprising:
a) contacting a sample of the subject with at least one polypeptide of the pathogen under conditions sufficient for an antibody to the at least one polypeptide of the pathogen to bind the at least one polypeptide of the pathogen to produce a bound antibody;
b) contacting the bound antibody with at least one recombinant antibody fusion protein to produce a binding complex, wherein the at least one recombinant antibody fusion protein is capable of binding to a human IgG and has a catalytic activity of converting at least one substrate into at least one detectable product;
c) contacting the binding complex with the at least one substrate under conditions sufficient to convert the at least one substrate into the at least one detectable product; and
d) detecting the presence or absence of the at least one detectable product in the sample of the subject, wherein detecting the presence of the at least one detectable product in the sample indicates that the subject has an infection of the pathogen,
optionally wherein the method further comprises obtaining the sample from the subject, optionally wherein the sample is an oral fluid, nasal fluid, saliva, blood, plasma, or serum, and optionally wherein the pathogen comprises a severe acute respiratory syndrome coronavirus-2 (SARS-COV-2).

8-10. (canceled)

11. A method for treating a pathogenic infection in a subject in need thereof, comprising:
a) diagnosing the subject as having the pathogenic infection by the method of claim 7; and
b) administering to the subject a therapeutically effective amount of treatment for the pathogenic infection.

12. (canceled)

13. The method of claim 1, wherein the at least one recombinant antibody fusion protein comprises:
a) at least one polypeptide comprising an anti-human immunoglobin (IgG) antibody fragment fused to an invertase, and a linker positioned between the anti-human IgG antibody fragment and the invertase, wherein the anti-human IgG antibody fragment comprises an amino acid sequence having at least 90% sequence identity to the amino acid sequence of SEQ ID NO: 2 or SEQ ID NO: 4 and the invertase comprises an amino acid sequence having at least 90% sequence identity to the amino acid sequence of SEQ ID NO: 6;
b) at least one polypeptide comprising an amino acid sequence having at least 90% sequence identity to the amino acid sequence of SEQ ID NO: 26, SEQ ID NO: 28, SEQ ID NO: 30, or SEQ ID NO: 32; or
c) at least one polypeptide comprising the amino acid sequence of SEQ ID NO: 26, SEQ ID NO: 28, SEQ ID NO: 30, or SEQ ID NO: 32.

14. The method of claim 13, wherein:
a) the at least one recombinant antibody fusion protein comprises 2 copies of the at least one polypeptide;
b) the linker comprises an amino acid sequence having at least 90% sequence identity to the amino acid sequence of SEQ ID NO: 8, SEQ ID NO: 10, SEQ ID NO: 12, or SEQ ID NO: 14; and/or
c) the at least one polypeptide further comprises a signal peptide positioned at the N-terminus of the polypeptide, optionally wherein the signal peptide comprises an amino acid sequence having at least 90% sequence identity to the amino acid sequence of SEQ ID NO: 16, SEQ ID NO: 18, or SEQ ID NO: 20.

15-19. (canceled)

20. The method of claim 1, wherein the at least one recombinant antibody fusion protein comprises:
a) 2 copies of a polypeptide comprising the amino acid sequence of SEQ ID NO: 26 and 2 copies of an anti-human IgG antibody fragment comprising the amino acid sequence of SEQ ID NO: 24;
b) 2 copies of a polypeptide comprising the amino acid sequence of SEQ ID NO: 28 and 2 copies of an anti-human IgG antibody fragment comprising the amino acid sequence of SEQ ID NO: 24;
c) 2 copies of a polypeptide comprising the amino acid sequence of SEQ ID NO: 30 and 2 copies of an anti-human IgG antibody fragment comprising the amino acid sequence of SEQ ID NO: 22; or
d) 2 copies of a polypeptide comprising the amino acid sequence of SEQ ID NO: 32 and 2 copies of an anti-human IgG antibody fragment comprising the amino acid sequence of SEQ ID NO: 22.

21. The method of claim 1, wherein;
a) the at least one substrate comprises sucrose and the at least one detectable product comprises glucose;
b) the presence of the at least one detectable product in the sample is detected using a glucometer;
c) the method is not conducted in a hospital or laboratory setting; and/or
d) the method is conducted as a point-of-care diagnostic test.

22-24. (canceled)

25. A polypeptide comprising an anti-human immunoglobin (IgG) antibody fragment fused to an invertase, and a linker positioned between the anti-human IgG antibody fragment and the invertase, wherein the anti-human IgG antibody fragment comprises an amino acid sequence having at least 90% sequence identity to the amino acid sequence of SEQ ID NO: 2 or SEQ ID NO: 4 and the invertase comprises an amino acid sequence having at least 90% sequence identity to the amino acid sequence of SEQ ID NO: 6.

26. The polypeptide of claim 25, wherein:
a) the linker comprises an amino acid sequence having at least 90% sequence identity to the amino acid sequence of SEQ ID NO: 8, SEQ ID NO: 10, SEQ ID NO: 12, or SEQ ID NO: 14; and/or
b) the polypeptide further comprises a signal peptide positioned at the N-terminus of the polypeptide, optionally wherein the signal peptide comprises an amino acid sequence having at least 90% sequence identity to the amino acid sequence of SEQ ID NO: 16, SEQ ID NO: 18, or SEQ ID NO: 20.

27-28. (canceled)

29. The polypeptide of claim 25, wherein the polypeptide comprises:
a) an amino acid sequence having at least 90% sequence identity to the amino acid sequence of SEQ ID NO: 26, SEQ ID NO: 28, SEQ ID NO: 30, or SEQ ID NO: 32; or
b) the amino acid sequence of SEQ ID NO: 26, SEQ ID NO: 28, SEQ ID NO: 30, or SEQ ID NO: 32.

30. (canceled)

31. A nucleic acid molecule encoding the polypeptide of claim 25, a vector comprising said nucleic acid molecule, or a host cell comprising said nucleic acid molecule or said vector.

32-33. (canceled)

34. A recombinant antibody fusion protein comprising at least one polypeptide of claim 25, optionally wherein the recombinant antibody fusion protein comprises 2 copies of the at least one polypeptide.

35. (canceled)

36. The recombinant antibody fusion protein of claim 34, comprising:
a) 2 copies of the polypeptide comprising the amino acid sequence of SEQ ID NO: 26 and 2 copies of the anti-human IgG antibody fragment comprising the amino acid sequence of SEQ ID NO: 24;
b) 2 copies of the polypeptide comprising the amino acid sequence of SEQ ID NO: 28 and 2 copies of the anti-human IgG antibody fragment comprising the amino acid sequence of SEQ ID NO: 24;
c) 2 copies of the polypeptide comprising the amino acid sequence of SEQ ID NO: 30 and 2 copies of the anti-human IgG antibody fragment comprising the amino acid sequence of SEQ ID NO: 22; or
d) 2 copies of the polypeptide comprising the amino acid sequence of SEQ ID NO: 32 and 2 copies of the anti-human IgG antibody fragment comprising the amino acid sequence of SEQ ID NO: 22.

37. The recombinant antibody fusion protein of claim 34, wherein the recombinant antibody fusion protein is capable of binding to a human IgG and has a catalytic activity of converting sucrose to glucose.

38. A kit comprising an antigenic polypeptide immobilized or absorbed onto a solid support and at least one recombinant antibody fusion protein of claim 34, optionally wherein the antigenic polypeptide is a pathogen-specific polypeptide or a polypeptide of a severe acute respiratory syndrome coronavirus-2 (SARS-COV-2) and/or the solid support is a strip, bead, plate, or slide, optionally wherein the kit is a point-of-care diagnostic kit and/or an over-the-counter diagnostic kit.

39-41. (canceled)

42. The kit of claim 38, further comprising a solution comprising sucrose and/or a glucometer.

43-45. (canceled)

46. A method of detecting an antibody to an antigenic polypeptide in a sample, the method comprising detecting the presence or absence of the antibody in the sample using the kit of claim 38, optionally wherein the antigenic polypeptide is:
a) a polypeptide of an infectious pathogen;
b) a polypeptide of a severe acute respiratory syndrome coronavirus-2 (SARS-COV-2); or
c) a polypeptide of human origin.

47-49. (canceled)

50. A method of diagnosing a pathogenic infection in a subject in need thereof, comprising detecting the presence or absence of an antibody to at least one pathogen-specific polypeptide in a sample of the subject using the kit of claim 38, wherein, when the pathogenic infection is caused by severe acute respiratory syndrome coronavirus-2 (SARS-COV-2), the at least one pathogen-specific polypeptide is the receptor binding domain (RBD) of the SARS-COV-2 spike protein.

51. (canceled)

52. A method of treating a pathogenic infection in a subject in need thereof, comprising detecting the presence or absence of an antibody to at least one pathogen-specific polypeptide in a sample of the subject using the kit of claim 38 and administering to the subject a therapeutically effective amount of treatment for the pathogenic infection if the presence of the at least one pathogen-specific polypeptide is detected.

53. (canceled)


SEQUENCE LISTING (34 sequences)

SEQ ID NO: 1 - DNA, 1341 bp, unidentified
  source 1..1341: mol_type = other DNA
caggtccaactgcagcagcctggggctgagcttgtgaagcctggggcttcagtgaagatgtcctgcaaggctagcggctacaccttcaccaactactggataaactgggtgaagcagaggcctggacaaggtctagagtggattggagatatttatcctggtggaggtattactaattacaatgagaagttcaagaccaaggccacactgactctggacacatcctcgtcgacagtctatatgcaactcagcagcctgacatctgaagactctgcggtatactattgttcaagatcctatggtaaatatttcgactactggggccaaggcaccactctcatagtctcctcagccaaaacaacggcgccatcggtctatccactggcccctgtgtgtggagatacaactggctcgtcggtgactctaggatgcctggtcaagggttatttccctgagccagtgaccttgacctggaactctggttccctgtccagtggtgtgcacaccttcccagctgtcctccagtctgacctctacaccctcagcagctcagtgactgtaacctcgagcacctggcccagccagtccatcacctgcaatgtggcccacccggcaagcagcaccaaggtggacaagaaaattgagcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaa

SEQ ID NO: 2 - protein, 447 aa, unidentified
QVQLQQPGAELVKPGASVKMSCKASGYTFTNYWINWVKQRPGQGLEWIGDIYPGGGITNYNEKFKTKATLTLDTSSSTVYMQLSSLTSEDSAVYYCSRSYGKYFDYWGQGTTLIVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 3 - DNA, 642 bp, unidentified
  source 1..642: mol_type = other DNA
gatatcaagatgacccagtccccatcctccatgtatgcatcggtgggagagagagtcactatcacgtgcaaggcgagtcaggacattaaaagctatttaacctggtaccagaagaagccatggaaatctcctaggaccctgatcttctattcaacaaggttggcagatggggtcccatcaagattcagtggcagtggatctgggcaagatttttctctaaccatcagcagcctggagtctgacgatacagcaacttattactgtctccagcatgatgagagcccgttcacgttcggaggagggaccaagctggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgt

SEQ ID NO: 4 - protein, 214 aa, unidentified
DIKMTQSPSSMYASVGERVTITCKASQDIKSYLTWYQKKPWKSPRTLIFYSTRLADGVPSRFSGSGSGQDFSLTISSLESDDTATYYCLQHDESPFTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 5 - DNA, 1536 bp, Saccharomyces cerevisiae
  source 1..1536: mol_type = genomic DNA
atgacaaacgaaactagcgatagacctttggtccacttcacacccaacaagggctggatgaatgacccaaatgggttgtggtacgatgaaaaagatgccaaatggcatctgtactttcaatacaacccaaatgacaccgtatggggtacgccattgttttggggccatgctacttccgatgatttgactaattgggaagatcaacccattgctatcgctcccaagcgtaacgattcaggtgctttctctggctccatggtggttgattacaacaacacgagtgggtttttcaatgatactattgatccaagacaaagatgcgttgcgatttggacttataacactcctgaaagtgaagagcaatacattagctattctcttgatggtggttacacttttactgaataccaaaagaaccctgttttagctgccaactccactcaattcagagatccaaaggtgttctggtatgaaccttctcaaaaatggattatgacggctgccaaatcacaagactacaaaattgaaatttactcctctgatgacttgaagtcctggaagctagaatctgcatttgccaatgaaggtttcttaggctaccaatacgaatgtccaggtttgattgaagtcccaactgagcaagatccttccaaatcttattgggtcatgtttatttctatcaacccaggtgcacctgctggcggttccttcaaccaatattttgttgggtccttcaatggtactcattttgaagcgtttgacaatcaatctagagtggtagattttggtaaggactactatgccttgcaaactttcttcaacactgacccaacctacggttcagcattaggtattgcctgggcttcaaactgggagtacagtgcctttgtcccaactaacccatggagatcatccatgtctttggtccgcaagttttctttgaacactgaatatcaagctaatccagagactgaattgatcaatttgaaagccgaaccaatattgaacattagtaatgctggtccctggtctcgttttgctactaacacaactctaactaaggccaattcttacaatgtcgatttgagcaactcgactggtaccctagagtttgagttggtttacgctgttaacaccacacaaaccatatccaaatccgtctttgccgacttatcactttggttcaagggtttagaagatcctgaagaatatttgagaatgggttttgaagtcagtgcttcttccttctttttggaccgtggtaactctaaggtcaagtttgtcaaggagaacccatatttcacaaacagaatgtctgtcaacaaccaaccattcaagtctgagaacgacctaagttactataaagtgtacggcctactggatcaaaacatcttggaattgtacttcaacgatggagatgtggtttctacaaatacctacttcatgaccaccggtaacgctctaggatctgtgaacatgaccactggtgtcgataatttgttctacattgacaagttccaagtaagggaagtaaaa

SEQ ID NO: 6 - protein, 512 aa, Saccharomyces cerevisiae
MTNETSDRPLVHFTPNKGWMNDPNGLWYDEKDAKWHLYFQYNPNDTVWGTPLFWGHATSDDLTNWEDQPIAIAPKRNDSGAFSGSMVVDYNNTSGFFNDTIDPRQRCVAIWTYNTPESEEQYISYSLDGGYTFTEYQKNPVLAANSTQFRDPKVFWYEPSQKWIMTAAKSQDYKIEIYSSDDLKSWKLESAFANEGFLGYQYECPGLIEVPTEQDPSKSYWVMFISINPGAPAGGSFNQYFVGSFNGTHFEAFDNQSRVVDFGKDYYALQTFFNTDPTYGSALGIAWASNWEYSAFVPTNPWRSSMSLVRKFSLNTEYQANPETELINLKAEPILNISNAGPWSRFATNTTLTKANSYNVDLSNSTGTLEFELVYAVNTTQTISKSVFADLSLWFKGLEDPEEYLRMGFEVSASSFFLDRGNSKVKFVKENPYFTNRMSVNNQPFKSENDLSYYKVYGLLDQNILELYFNDGDVVSTNTYFMTTGNALGSVNMTTGVDNLFYIDKFQVREVK

SEQ ID NO: 7 - DNA, 45 bp, synthetic construct
  source 1..45: mol_type = other DNA
ggtggtggaggttcagggggtggaggtagcggcggagggggatcc

SEQ ID NO: 8 - protein, 15 aa, synthetic construct
GGGGSGGGGSGGGGS

SEQ ID NO: 9 - DNA, 51 bp, synthetic construct
  source 1..51: mol_type = other DNA
gctagcggtggtggaggttcaggcgggggcggtagcggcggtgggggatcc

SEQ ID NO: 10 - protein, 17 aa, synthetic construct
ASGGGGSGGGGSGGGGS

SEQ ID NO: 11 - DNA, 75 bp, synthetic construct
  source 1..75: mol_type = other DNA
ggtggtggaggttcagggggtggaggatctggcggagggggttcaggcgggggcggtagcggcggtgggggatcc

SEQ ID NO: 12 - protein, 25 aa, synthetic construct
GGGGSGGGGSGGGGSGGGGSGGGGS

SEQ ID NO: 13 - DNA, 81 bp, synthetic construct
  source 1..81: mol_type = other DNA
gctagcggtggtggaggttcagggggtggaggatctggcggagggggttcaggcgggggcggtagcggcggtgggggatcc

SEQ ID NO: 14 - protein, 27 aa, synthetic construct
ASGGGGSGGGGSGGGGSGGGGSGGGGS

SEQ ID NO: 15 - DNA, 57 bp, synthetic construct
  source 1..57: mol_type = other DNA
atgggatggagctgtatcatcctctttttggtagcaacagctacaggtgtccactcc

SEQ ID NO: 16 - protein, 19 aa, synthetic construct
MGWSCIILFLVATATGVHS

SEQ ID NO: 17 - DNA, 60 bp, synthetic construct
  source 1..60: mol_type = other DNA
atgagggcccctgctcagttttttgggatcttgttgctctggtttccaggtatcagatgt

SEQ ID NO: 18 - protein, 20 aa, synthetic construct
MRAPAQFFGILLLWFPGIRC

SEQ ID NO: 19 - DNA, 60 bp, synthetic construct
  source 1..60: mol_type = other DNA
atgagggtccccgctcagctcctggggctcctgctgctctggctcccaggtgcacgatgt

SEQ ID NO: 20 - protein, 20 aa, synthetic construct
MRVPAQLLGLLLLWLPGARC

SEQ ID NO: 21 - DNA, 1398 bp, synthetic construct
  source 1..1398: mol_type = other DNA
atgggatggagctgtatcatcctctttttggtagcaacagctacaggtgtccactcccaggtccaactgcagcagcctggggctgagcttgtgaagcctggggcttcagtgaagatgtcctgcaaggctagcggctacaccttcaccaactactggataaactgggtgaagcagaggcctggacaaggtctagagtggattggagatatttatcctggtggaggtattactaattacaatgagaagttcaagaccaaggccacactgactctggacacatcctcgtcgacagtctatatgcaactcagcagcctgacatctgaagactctgcggtatactattgttcaagatcctatggtaaatatttcgactactggggccaaggcaccactctcatagtctcctcagccaaaacaacggcgccatcggtctatccactggcccctgtgtgtggagatacaactggctcgtcggtgactctaggatgcctggtcaagggttatttccctgagccagtgaccttgacctggaactctggttccctgtccagtggtgtgcacaccttcccagctgtcctccagtctgacctctacaccctcagcagctcagtgactgtaacctcgagcacctggcccagccagtccatcacctgcaatgtggcccacccggcaagcagcaccaaggtggacaagaaaattgagcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaa

SEQ ID NO: 22 - protein, 466 aa, synthetic construct
MGWSCIILFLVATATGVHSQVQLQQPGAELVKPGASVKMSCKASGYTFTNYWINWVKQRPGQGLEWIGDIYPGGGITNYNEKFKTKATLTLDTSSSTVYMQLSSLTSEDSAVYYCSRSYGKYFDYWGQGTTLIVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 23 - DNA, 702 bp, synthetic construct
  source 1..702: mol_type = other DNA
atgagggcccctgctcagttttttgggatcttgttgctctggtttccaggtatcagatgtgatatcaagatgacccagtccccatcctccatgtatgcatcggtgggagagagagtcactatcacgtgcaaggcgagtcaggacattaaaagctatttaacctggtaccagaagaagccatggaaatctcctaggaccctgatcttctattcaacaaggttggcagatggggtcccatcaagattcagtggcagtggatctgggcaagatttttctctaaccatcagcagcctggagtctgacgatacagcaacttattactgtctccagcatgatgagagcccgttcacgttcggaggagggaccaagctggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgt

SEQ ID NO: 24 - protein, 234 aa, synthetic construct
MRAPAQFFGILLLWFPGIRCDIKMTQSPSSMYASVGERVTITCKASQDIKSYLTWYQKKPWKSPRTLIFYSTRLADGVPSRFSGSGSGQDFSLTISSLESDDTATYYCLQHDESPFTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 25 - DNA, 2985 bp, synthetic construct
  source 1..2985: mol_type = other DNA
atgggatggagctgtatcatcctctttttggtagcaacagctacaggtgtccactcccaggtccaactgcagcagcctggggctgagcttgtgaagcctggggcttcagtgaagatgtcctgcaaggctagcggctacaccttcaccaactactggataaactgggtgaagcagaggcctggacaaggtctagagtggattggagatatttatcctggtggaggtattactaattacaatgagaagttcaagaccaaggccacactgactctggacacatcctcgtcgacagtctatatgcaactcagcagcctgacatctgaagactctgcggtatactattgttcaagatcctatggtaaatatttcgactactggggccaaggcaccactctcatagtctcctcagccaaaacaacggcgccatcggtctatccactggcccctgtgtgtggagatacaactggctcgtcggtgactctaggatgcctggtcaagggttatttccctgagccagtgaccttgacctggaactctggttccctgtccagtggtgtgcacaccttcccagctgtcctccagtctgacctctacaccctcagcagctcagtgactgtaacctcgagcacctggcccagccagtccatcacctgcaatgtggcccacccggcaagcagcaccaaggtggacaagaaaattgagcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaagctagcggtggtggaggttcaggcgggggcggtagcggcggtgggggatccatgacaaacgaaactagcgatagacctttggtccacttcacacccaacaagggctggatgaatgacccaaatgggttgtggtacgatgaaaaagatgccaaatggcatctgtactttcaatacaacccaaatgacaccgtatggggtacgccattgttttggggccatgctacttccgatgatttgactaattgggaagatcaacccattgctatcgctcccaagcgtaacgattcaggtgctttctctggctccatggtggttgattacaacaacacgagtgggtttttcaatgatactattgatccaagacaaagatgcgttgcgatttggacttataacactcctgaaagtgaagagcaatacattagctattctcttgatggtggttacacttttactgaataccaaaagaaccctgttttagctgccaactccactcaattcagagatccaaaggtgttctggtatgaaccttctcaaaaatggattatgacggctgccaaatcacaagactacaaaattgaaatttactcctctgatgacttgaagtcctggaagctagaatctgcatttgccaatgaaggtttcttaggctaccaatacgaatgtccaggtttgattgaagtcccaactgagcaagatccttccaaatcttattgggtcatgtttatttctatcaacccaggtgcacctgctggcggttccttcaaccaatattttgttgggtccttcaatggtactcattttgaagcgtttgacaatcaatctagagtggtagattttggtaaggactactatgccttgcaaactttcttcaacactgacccaacatacggttcagcattaggtattgcctgggcttcaaactgggagtacagtgcctttgtcccaactaacccatggagatcatccatgtctttggtccgcaagttttctttgaacactgaatatcaagctaatccagagactgaattgatcaatttgaaagccgaaccaatattgaacattagtaatgctggtccctggtctcgttttgctactaacacaactctaactaaggccaattcttacaatgtcgatttgagcaactcgactggtaccctagagtttgagttggtttacgctgttaacaccacacaaaccatatccaaatccgtctttgccgacttatcactttggttcaagggtttagaagatcctgaagaatatttgagaatgggttttgaagtcagtgcttcttccttctttttggaccgtggtaactctaaggtcaagtttgtcaaggagaacccatatttcacaaacagaatgtctgtcaacaaccaaccattcaagtctgagaacgacctaagttactataaagtgtacggcctactggatcaaaacatcttggaattgtacttcaacgatggagatgtggtttctacaaatacctacttcatgaccaccggtaacgctctaggatctgtgaacatgaccactggtgtcgataatttgttctacattgacaagttccaagtaagggaagtaaaa

SEQ ID NO: 26 - protein, 995 aa, synthetic construct
MGWSCIILFLVATATGVHSQVQLQQPGAELVKPGASVKMSCKASGYTFTNYWINWVKQRPGQGLEWIGDIYPGGGITNYNEKFKTKATLTLDTSSSTVYMQLSSLTSEDSAVYYCSRSYGKYFDYWGQGTTLIVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKASGGGGSGGGGSGGGGSMTNETSDRPLVHFTPNKGWMNDPNGLWYDEKDAKWHLYFQYNPNDTVWGTPLFWGHATSDDLTNWEDQPIAIAPKRNDSGAFSGSMVVDYNNTSGFFNDTIDPRQRCVAIWTYNTPESEEQYISYSLDGGYTFTEYQKNPVLAANSTQFRDPKVFWYEPSQKWIMTAAKSQDYKIEIYSSDDLKSWKLESAFANEGFLGYQYECPGLIEVPTEQDPSKSYWVMFISINPGAPAGGSFNQYFVGSFNGTHFEAFDNQSRVVDFGKDYYALQTFFNTDPTYGSALGIAWASNWEYSAFVPTNPWRSSMSLVRKFSLNTEYQANPETELINLKAEPILNISNAGPWSRFATNTTLTKANSYNVDLSNSTGTLEFELVYAVNTTQTISKSVFADLSLWFKGLEDPEEYLRMGFEVSASSFFLDRGNSKVKFVKENPYFTNRMSVNNQPFKSENDLSYYKVYGLLDQNILELYFNDGDVVSTNTYFMTTGNALGSVNMTTGVDNLFYIDKFQVREVK

SEQ ID NO: 27 - skipped

SEQ ID NO: 28 - protein, 1005 aa, synthetic construct
MGWSCIILFLVATATGVHSQVQLQQPGAELVKPGASVKMSCKASGYTFTNYWINWVKQRPGQGLEWIGDIYPGGGITNYNEKFKTKATLTLDTSSSTVYMQLSSLTSEDSAVYYCSRSYGKYFDYWGQGTTLIVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKASGGGGSGGGGSGGGGSGGGGSGGGGSMTNETSDRPLVHFTPNKGWMNDPNGLWYDEKDAKWHLYFQYNPNDTVWGTPLFWGHATSDDLTNWEDQPIAIAPKRNDSGAFSGSMVVDYNNTSGFFNDTIDPRQRCVAIWTYNTPESEEQYISYSLDGGYTFTEYQKNPVLAANSTQFRDPKVFWYEPSQKWIMTAAKSQDYKIEIYSSDDLKSWKLESAFANEGFLGYQYECPGLIEVPTEQDPSKSYWVMFISINPGAPAGGSFNQYFVGSFNGTHFEAFDNQSRVVDFGKDYYALQTFFNTDPTYGSALGIAWASNWEYSAFVPTNPWRSSMSLVRKFSLNTEYQANPETELINLKAEPILNISNAGPWSRFATNTTLTKANSYNVDLSNSTGTLEFELVYAVNTTQTISKSVFADLSLWFKGLEDPEEYLRMGFEVSASSFFLDRGNSKVKFVKENPYFTNRMSVNNQPFKSENDLSYYKVYGLLDQNILELYFNDGDVVSTNTYFMTTGNALGSVNMTTGVDNLFYIDKFQVREVK

SEQ ID NO: 29 - DNA, 2283 bp, synthetic construct
  source 1..2283: mol_type = other DNA
atgagggcccctgctcagttttttgggatcttgttgctctggtttccaggtatcagatgtgatatcaagatgacccagtccccatcctccatgtatgcatcggtgggagagagagtcactatcacgtgcaaggcgagtcaggacattaaaagctatttaacctggtaccagaagaagccatggaaatctcctaggaccctgatcttctattcaacaaggttggcagatggggtcccatcaagattcagtggcagtggatctgggcaagatttttctctaaccatcagcagcctggagtctgacgatacagcaacttattactgtctccagcatgatgagagcccgttcacgttcggaggagggaccaagctggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgtggtggtggaggttcagggggtggaggtagcggcggagggggatccatgacaaacgaaactagcgatagacctttggtccacttcacacccaacaagggctggatgaatgacccaaatgggttgtggtacgatgaaaaagatgccaaatggcatctgtactttcaatacaacccaaatgacaccgtatggggtacgccattgttttggggccatgctacttccgatgatttgactaattgggaagatcaacccattgctatcgctcccaagcgtaacgattcaggtgctttctctggctccatggtggttgattacaacaacacgagtgggtttttcaatgatactattgatccaagacaaagatgcgttgcgatttggacttataacactcctgaaagtgaagagcaatacattagctattctcttgatggtggttacacttttactgaataccaaaagaaccctgttttagctgccaactccactcaattcagagatccaaaggtgttctggtatgaaccttctcaaaaatggattatgacggctgccaaatcacaagactacaaaattgaaatttactcctctgatgacttgaagtcctggaagctagaatctgcatttgccaatgaaggtttcttaggctaccaatacgaatgtccaggtttgattgaagtcccaactgagcaagatccttccaaatcttattgggtcatgtttatttctatcaacccaggtgcacctgctggcggttccttcaaccaatattttgttgggtccttcaatggtactcattttgaagcgtttgacaatcaatctagagtggtagattttggtaaggactactatgccttgcaaactttcttcaacactgacccaacctacggttcagcattaggtattgcctgggcttcaaactgggagtacagtgcctttgtcccaactaacccatggagatcatccatgtctttggtccgcaagttttctttgaacactgaatatcaagctaatccagagactgaattgatcaatttgaaagccgaaccaatattgaacattagtaatgctggtccctggtctcgttttgctactaacacaactctaactaaggccaattcttacaatgtcgatttgagcaactcgactggtaccctagagtttgagttggtttacgctgttaacaccacacaaaccatatccaaatccgtctttgccgacttatcactttggttcaagggtttagaagatcctgaagaatatttgagaatgggttttgaagtcagtgcttcttccttctttttggaccgtggtaactctaaggtcaagtttgtcaaggagaacccatatttcacaaacagaatgtctgtcaacaaccaaccattcaagtctgagaacgacctaagttactataaagtgtacggcctactggatcaaaacatcttggaattgtacttcaacgatggagatgtggtttctacaaatacctacttcatgaccaccggtaacgctctaggatctgtgaacatgaccactggtgtcgataatttgttctacattgacaagttccaagtaagggaagtaaaa

SEQ ID NO: 30 - protein, 761 aa, synthetic construct
MRAPAQFFGILLLWFPGIRCDIKMTQSPSSMYASVGERVTITCKASQDIKSYLTWYQKKPWKSPRTLIFYSTRLADGVPSRFSGSGSGQDFSLTISSLESDDTATYYCLQHDESPFTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNECGGGGSGGGGSGGGGSMTNETSDRPLVHFTPNKGWMNDPNGLWYDEKDAKWHLYFQYNPNDTVWGTPLFWGHATSDDLTNWEDQPIAIAPKRNDSGAFSGSMVVDYNNTSGFFNDTIDPRQRCVAIWTYNTPESEEQYISYSLDGGYTFTEYQKNPVLAANSTQFRDPKVFWYEPSQKWIMTAAKSQDYKIEIYSSDDLKSWKLESAFANEGFLGYQYECPGLIEVPTEQDPSKSYWVMFISINPGAPAGGSFNQYFVGSFNGTHFEAFDNQSRVVDFGKDYYALQTFFNTDPTYGSALGIAWASNWEYSAFVPTNPWRSSMSLVRKFSLNTEYQANPETELINLKAEPILNISNAGPWSRFATNTTLTKANSYNVDLSNSTGTLEFELVYAVNTTQTISKSVFADLSLWFKGLEDPEEYLRMGFEVSASSFFLDRGNSKVKFVKENPYFTNRMSVNNQPFKSENDLSYYKVYGLLDQNILELYFNDGDVVSTNTYFMTTGNALGSVNMTTGVDNLFYIDKFQVREVK

SEQ ID NO: 31 - DNA, 2313 bp, synthetic construct
  source 1..2313: mol_type = other DNA
atgagggcccctgctcagttttttgggatcttgttgctctggtttccaggtatcagatgtgatatcaagatgacccagtccccatcctccatgtatgcatcggtgggagagagagtcactatcacgtgcaaggcgagtcaggacattaaaagctatttaacctggtaccagaagaagccatggaaatctcctaggaccctgatcttctattcaacaaggttggcagatggggtcccatcaagattcagtggcagtggatctgggcaagatttttctctaaccatcagcagcctggagtctgacgatacagcaacttattactgtctccagcatgatgagagcccgttcacgttcggaggagggaccaagctggaaataaaacgggctgatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgtggtggtggaggttcagggggtggaggatctggcggagggggttcaggcgggggcggtagcggcggtgggggatccatgacaaacgaaactagcgatagacctttggtccacttcacacccaacaagggctggatgaatgacccaaatgggttgtggtacgatgaaaaagatgccaaatggcatctgtactttcaatacaacccaaatgacaccgtatggggtacgccattgttttggggccatgctacttccgatgatttgactaattgggaagatcaacccattgctatcgctcccaagcgtaacgattcaggtgctttctctggctccatggtggttgattacaacaacacgagtgggtttttcaatgatactattgatccaagacaaagatgcgttgcgatttggacttataacactcctgaaagtgaagagcaatacattagctattctcttgatggtggttacacttttactgaataccaaaagaaccctgttttagctgccaactccactcaattcagagatccaaaggtgttctggtatgaaccttctcaaaaatggattatgacggctgccaaatcacaagactacaaaattgaaatttactcctctgatgacttgaagtcctggaagctagaatctgcatttgccaatgaaggtttcttaggctaccaatacgaatgtccaggtttgattgaagtcccaactgagcaagatccttccaaatcttattgggtcatgtttatttctatcaacccaggtgcacctgctggcggttccttcaaccaatattttgttgggtccttcaatggtactcattttgaagcgtttgacaatcaatctagagtggtagattttggtaaggactactatgccttgcaaactttcttcaacactgacccaacctacggttcagcattaggtattgcctgggcttcaaactgggagtacagtgcctttgtcccaactaacccatggagatcatccatgtctttggtccgcaagttttctttgaacactgaatatcaagctaatccagagactgaattgatcaatttgaaagccgaaccaatattgaacattagtaatgctggtccctggtctcgttttgctactaacacaactctaactaaggccaattcttacaatgtcgatttgagcaactcgactggtaccctagagtttgagttggtttacgctgttaacaccacacaaaccatatccaaatccgtctttgccgacttatcactttggttcaagggtttagaagatcctgaagaatatttgagaatgggttttgaagtcagtgcttcttccttctttttggaccgtggtaactctaaggtcaagtttgtcaaggagaacccatatttcacaaacagaatgtctgtcaacaaccaaccattcaagtctgagaacgacctaagttactataaagtgtacggcctactggatcaaaacatcttggaattgtacttcaacgatggagatgtggtttctacaaatacctacttcatgaccaccggtaacgctctaggatctgtgaacatgaccactggtgtcgataatttgttctacattgacaagttccaagtaagggaagtaaaa

SEQ ID NO: 32 - protein, 771 aa, synthetic construct
MRAPAQFFGILLLWFPGIRCDIKMTQSPSSMYASVGERVTITCKASQDIKSYLTWYQKKPWKSPRTLIFYSTRLADGVPSRFSGSGSGQDFSLTISSLESDDTATYYCLQHDESPFTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNECGGGGSGGGGSGGGGSGGGGSGGGGSMTNETSDRPLVHFTPNKGWMNDPNGLWYDEKDAKWHLYFQYNPNDTVWGTPLFWGHATSDDLTNWEDQPIAIAPKRNDSGAFSGSMVVDYNNTSGFFNDTIDPRQRCVAIWTYNTPESEEQYISYSLDGGYTFTEYQKNPVLAANSTQFRDPKVFWYEPSQKWIMTAAKSQDYKIEIYSSDDLKSWKLESAFANEGFLGYQYECPGLIEVPTEQDPSKSYWVMFISINPGAPAGGSFNQYFVGSFNGTHFEAFDNQSRVVDFGKDYYALQTFFNTDPTYGSALGIAWASNWEYSAFVPTNPWRSSMSLVRKFSLNTEYQANPETELINLKAEPILNISNAGPWSRFATNTTLTKANSYNVDLSNSTGTLEFELVYAVNTTQTISKSVFADLSLWFKGLEDPEEYLRMGFEVSASSFFLDRGNSKVKFVKENPYFTNRMSVNNQPFKSENDLSYYKVYGLLDQNILELYFNDGDVVSTNTYFMTTGNALGSVNMTTGVDNLFYIDKFQVREVK

SEQ ID NO: 33 - DNA, 1623 bp, synthetic construct
  source 1..1623: mol_type = other DNA
atgagggtccccgctcagctcctggggctcctgctgctctggctcccaggtgcacgatgtatgacaaacgaaactagcgatagacctttggtccacttcacacccaacaagggctggatgaatgacccaaatgggttgtggtacgatgaaaaagatgccaaatggcatctgtactttcaatacaacccaaatgacaccgtatggggtacgccattgttttggggccatgctacttccgatgatttgactaattgggaagatcaacccattgctatcgctcccaagcgtaacgattcaggtgctttctctggctccatggtggttgattacaacaacacgagtgggtttttcaatgatactattgatccaagacaaagatgcgttgcgatttggacttataacactcctgaaagtgaagagcaatacattagctattctcttgatggtggttacacttttactgaataccaaaagaaccctgttttagctgccaactccactcaattcagagatccaaaggtgttctggtatgaaccttctcaaaaatggattatgacggctgccaaatcacaagactacaaaattgaaatttactcctctgatgacttgaagtcctggaagctagaatctgcatttgccaatgaaggtttcttaggctaccaatacgaatgtccaggtttgattgaagtcccaactgagcaagatccttccaaatcttattgggtcatgtttatttctatcaacccaggtgcacctgctggcggttccttcaaccaatattttgttgggtccttcaatggtactcattttgaagcgtttgacaatcaatctagagtggtagattttggtaaggactactatgccttgcaaactttcttcaacactgacccaacctacggttcagcattaggtattgcctgggcttcaaactgggagtacagtgcctttgtcccaactaacccatggagatcatccatgtctttggtccgcaagttttctttgaacactgaatatcaagctaatccagagactgaattgatcaatttgaaagccgaaccaatattgaacattagtaatgctggtccctggtctcgttttgctactaacacaactctaactaaggccaattcttacaatgtcgatttgagcaactcgactggtaccctagagtttgagttggtttacgctgttaacaccacacaaaccatatccaaatccgtctttgccgacttatcactttggttcaagggtttagaagatcctgaagaatatttgagaatgggttttgaagtcagtgcttcttccttctttttggaccgtggtaactctaaggtcaagtttgtcaaggagaacccatatttcacaaacagaatgtctgtcaacaaccaaccattcaagtctgagaacgacctaagttactataaagtgtacggcctactggatcaaaacatcttggaattgtacttcaacgatggagatgtggtttctacaaatacctacttcatgaccaccggtaacgctctaggatctgtgaacatgaccactggtgtcgataatttgttctacattgacaagttccaagtaagggaagtaaaagcggccgcgcaccatcaccaccatcac

SEQ ID NO: 34 - protein, 541 aa, synthetic construct
MRVPAQLLGLLLLWLPGARCMTNETSDRPLVHFTPNKGWMNDPNGLWYDEKDAKWHLYFQYNPNDTVWGTPLFWGHATSDDLTNWEDQPIAIAPKRNDSGAFSGSMVVDYNNTSGFFNDTIDPRQRCVAIWTYNTPESEEQYISYSLDGGYTFTEYQKNPVLAANSTQFRDPKVFWYEPSQKWIMTAAKSQDYKIEIYSSDDLKSWKLESAFANEGFLGYQYECPGLIEVPTEQDPSKSYWVMFISINPGAPAGGSFNQYFVGSFNGTHFEAFDNQSRVVDFGKDYYALQTFFNTDPTYGSALGIAWASNWEYSAFVPTNPWRSSMSLVRKFSLNTEYQANPETELINLKAEPILNISNAGPWSRFATNTTLTKANSYNVDLSNSTGTLEFELVYAVNTTQTISKSVFADLSLWFKGLEDPEEYLRMGFEVSASSFFLDRGNSKVKFVKENPYFTNRMSVNNQPFKSENDLSYYKVYGLLDQNILELYFNDGDVVSTNTYFMTTGNALGSVNMTTGVDNLFYIDKFQVREVKAAAHHHHHH